SEQUENCE LISTING (136 sequences)

SEQ ID NO: 1 - protein, 9 aa, Artificial Sequence
  LN Targeting Peptide
PTCAYGWCA

SEQ ID NO: 2 - protein, 9 aa, Artificial Sequence
  LN Targeting Peptide
WSCARPLCG

SEQ ID NO: 3 - protein, 9 aa, Artificial Sequence
  Lymphatic Channel Targeting Peptide
CGLTFKSLC

SEQ ID NO: 4 - protein, 10 aa, Artificial Sequence
  Aerosol delivery peptide
CAKSMGDIVC

SEQ ID NO: 5 - protein, 11 aa, Artificial Sequence
  RGD4C-targeting peptide
ACDCRGDCFCG

SEQ ID NO: 6 - DNA, 33 bp, Artificial Sequence
  RGD4C- targeting peptide
gcgtgtgattgtaggggggattgtttttgtggc

SEQ ID NO: 7 - DNA, 27 bp, Artificial Sequence
  LN-targeting peptide nucleic acid
ccgacctgtgcgtatggctggtgtgcg

SEQ ID NO: 8 - DNA, 27 bp, Artificial Sequence
  LN-targeting peptide nucleic acid
tggagctgtgcgcgcccgctgtgtggc

SEQ ID NO: 9 - DNA, 27 bp, Artificial Sequence
  Lymphatic channel-targeting peptide
tgtggcctgaccttcaaaagcctgtgt

SEQ ID NO: 10 - protein, 19 aa, Artificial Sequence
  SARS-CoV-2 S protein optimized epitope
CNGTNQGGGYGGGYQGYGC

SEQ ID NO: 11 - protein, 14 aa, Artificial Sequence
  SARS-CoV-2 S protein optimized epitope
CNTNQGGYGGYQYC

SEQ ID NO: 12 - protein, 20 aa, Artificial Sequence
  SARS-CoV-2 S protein optimized epitope
CYQYGGGGYGGQNTGGGGNC

SEQ ID NO: 13 - protein, 14 aa, Artificial Sequence
  SARS-CoV-2 S protein optimized epitope
CYQYGGYGQNTGNC

SEQ ID NO: 14 - protein, 20 aa, Artificial Sequence
  SARS-CoV-2 S protein optimized epitope
CGGLQYGGGGYGQTNGGGNC

SEQ ID NO: 15 - protein, 14 aa, Artificial Sequence
  SARS-CoV-2 S protein optimized epitope
CLQYGGYQTNGGNC

SEQ ID NO: 16 - protein, 20 aa, Artificial Sequence
  SARS-CoV-2 S protein optimized epitope
CNGGGNTQGYGYSQYGGGTC

SEQ ID NO: 17 - protein, 14 aa, Artificial Sequence
  SARS-CoV-2 S protein optimized epitope
CNGNTQYYSQYGTC

SEQ ID NO: 18 - protein, 14 aa, Artificial Sequence
  SARS-CoV-2 S protein optimized epitope
CHTNSWGGGTNNCC

SEQ ID NO: 19 - protein, 15 aa, Artificial Sequence
  SARS-CoV-2 S protein optimized epitope
CYSNNSGGTGGNEQC

SEQ ID NO: 20 - protein, 20 aa, Artificial Sequence
  SARS-CoV-2 S protein optimized epitope
CYGTQNGTGGGYGGTQNGTC

SEQ ID NO: 21 - protein, 20 aa, Artificial Sequence
  SARS-CoV-2 S protein optimized epitope
CNNSQGGGGGNNSQGGGGGC

SEQ ID NO: 22 - protein, 26 aa, Artificial Sequence
  SARS-CoV-2 S protein epitope (aa 336-361)
CPFGEVFNATRFASVYAWNRKRISNC

SEQ ID NO: 23 - protein, 13 aa, Artificial Sequence
  SARS-CoV-2 S protein epitope (aa 379-391)
CYGVSPTKLNDLC

SEQ ID NO: 24 - protein, 9 aa, Artificial Sequence
  SARS-CoV-2 S protein epitope (aa 480-488)
CNGVEGFNC

SEQ ID NO: 25 - protein, 10 aa, Artificial Sequence
  SARS-CoV-2 S protein epitope (aa 662-671)
CDIPIGAGIC

SEQ ID NO: 26 - protein, 23 aa, Artificial Sequence
  SARS-CoV-2 S protein epitope (aa 738-760)
CTMYICGDSTECSNLLLQYGSFC

SEQ ID NO: 27 - protein, 12 aa, Artificial Sequence
  SARS-CoV-2 S protein epitope (aa 1032-1043)
CVLGQSKRVDFC

SEQ ID NO: 28 - protein, 9 aa, Artificial Sequence
  Lung-targeting peptide
CGSPGWVRC

SEQ ID NO: 29 - protein, 9 aa, Artificial Sequence
  GRP78-targeting peptide
CSNTRVAPC

SEQ ID NO: 30 - protein, 8 aa, Artificial Sequence
  GRP78-targeting peptide
WIFPWIQL

SEQ ID NO: 31 - protein, 18 aa, Artificial Sequence
  SARS-CoV-2 Spike protein epitope (aa 553-570)
TESNKKFLPFQQFGRDIA

SEQ ID NO: 32 - protein, 18 aa, Artificial Sequence
  SARS-CoV-2 Spike protein epitope (aa 809-826)
PSKPSKRSFIEDLLFNKV

SEQ ID NO: 33 - protein, 18 aa, Artificial Sequence
  SARS-CoV-2 Spike protein epitope (aa 369-386)
YNSASFSTFKCYGVSPTK

SEQ ID NO: 34 - protein, 33 aa, Artificial Sequence
  SARS-CoV-2 S protein epitope (aa 129-161)
KVCEFQFCNDPFLGVYYHKNNKSWMESEFRVYS

SEQ ID NO: 35 - protein, 33 aa, Artificial Sequence
  SARS-CoV-2 S protein epitope (aa 252-284)
GDSSSGWTAGAAAYYVGYLQPRTFLLKYNENGT

SEQ ID NO: 36 - protein, 33 aa, Artificial Sequence
  SARS-CoV-2 S protein epitope (aa 339-371)
GEVFNATRFASVYAWNRKRISNCVADYSVLYNS

SEQ ID NO: 37 - protein, 33 aa, Artificial Sequence
  SARS-CoV-2 S protein epitope (aa 462-494)
KPFERDISTEIYQAGSTPCNGVEGFNCYFPLQS

SEQ ID NO: 38 - protein, 33 aa, Artificial Sequence
  SARS-CoV-2 S protein epitope (aa 673-705)
SYQTQTNSPRRARSVASQSIIAYTMSLGAENSV

SEQ ID NO: 39 - protein, 15 aa, Artificial Sequence
  SARS-CoV-2 S protein epitope (aa 166-180)
CTFEYVSQPFLMDLE

SEQ ID NO: 40 - protein, 15 aa, Artificial Sequence
  SARS-CoV-2 S protein epitope (aa 751-765)
NLLLQYGSFCTQLNR

SEQ ID NO: 41 - protein, 15 aa, Artificial Sequence
  SARS-CoV-2 S protein epitope (aa 866-880)
TDEMIAQYTSALLAG

SEQ ID NO: 42 - protein, 15 aa, Artificial Sequence
  SARS-CoV-2 S protein epitope (aa 801-815)
NFSQILPDPSKPSKR

SEQ ID NO: 43 - protein, 12 aa, Artificial Sequence
  SARS-CoV-2 S protein epitope (aa 553-564)
TESNKKFLPFQQ

SEQ ID NO: 44 - protein, 12 aa, Artificial Sequence
  SARS-CoV-2 S protein epitope (aa 577-588)
RDPQTLEILDIT

SEQ ID NO: 45 - protein, 16 aa, Artificial Sequence
  SARS-CoV-2 S protein epitope (aa 595-612)
VSVITPGTNTSNQVAV

SEQ ID NO: 46 - protein, 18 aa, Artificial Sequence
  SARS-CoV-2 S protein epitope (aa 625-642)
HADQLTPTWRVYSTGSNV

SEQ ID NO: 47 - protein, 24 aa, Artificial Sequence
  SARS-CoV-2 S protein epitope (aa 661-684)
ECDIPIGAGICASYQTQTNSPRRA

SEQ ID NO: 48 - protein, 12 aa, Artificial Sequence
  SARS-CoV-2 S protein epitope (aa 1148-1159)
FKEELDKYFKNH

SEQ ID NO: 49 - protein, 25 aa, Artificial Sequence
  SARS-CoV-2 S protein epitope (aa 21-45)
RTQLPPAYTNSFTRGVYYPDKVFRS

SEQ ID NO: 50 - protein, 25 aa, Artificial Sequence
  SARS-CoV-2 S protein epitope (aa 221-245)
SALEPLVDLPIGINITRFQTLLALH

SEQ ID NO: 51 - protein, 25 aa, Artificial Sequence
  SARS-CoV-2 S protein epitope (aa 261-285)
GAAAYYVGYLQPRTFLLKYNENGTI

SEQ ID NO: 52 - protein, 20 aa, Artificial Sequence
  SARS-CoV-2 S protein epitope (aa 330-349)
PNITNLCPFGEVFNATRFAS

SEQ ID NO: 53 - protein, 25 aa, Artificial Sequence
  SARS-CoV-2 S protein epitope (aa 370-394)
NSASFSTFKCYGVSPTKLNDLCFTN

SEQ ID NO: 54 - protein, 20 aa, Artificial Sequence
  SARS-CoV-2 S protein epitope (aa 375-394)
STFKCYGVSPTKLNDLCFTN

SEQ ID NO: 55 - protein, 12 aa, Artificial Sequence
  SARS-CoV-2 S protein epitope (aa 406-417)
EVRQIAPGQTGK

SEQ ID NO: 56 - protein, 14 aa, Artificial Sequence
  SARS-CoV-2 S protein epitope (aa 414-427)
QTGKIADYNYKLPD

SEQ ID NO: 57 - protein, 13 aa, Artificial Sequence
  SARS-CoV-2 S protein epitope (aa 418-430)
IADYNYKLPDDFT

SEQ ID NO: 58 - protein, 5 aa, Artificial Sequence
  SARS-CoV-2 S protein epitope (aa 424-428)
KLPDD

SEQ ID NO: 59 - protein, 11 aa, Artificial Sequence
  SARS-CoV-2 S protein epitope (aa 438-448)
SNNLDSKVGGN

SEQ ID NO: 60 - protein, 20 aa, Artificial Sequence
  SARS-CoV-2 S protein epitope (aa 450-469)
NYLYRLFRKSNLKPFERDIS

SEQ ID NO: 61 - protein, 10 aa, Artificial Sequence
  SARS-CoV-2 S protein epitope (aa 454-463)
RLFRKSNLKP

SEQ ID NO: 62 - protein, 9 aa, Artificial Sequence
  SARS-CoV-2 S protein epitope (aa 459-467)
SNLKPFERD

SEQ ID NO: 63 - protein, 11 aa, Artificial Sequence
  SARS-CoV-2 S protein epitope (aa 478-488)
TPCNGVEGFNC

SEQ ID NO: 64 - protein, 20 aa, Artificial Sequence
  SARS-CoV-2 S protein epitope (aa 480-499)
CNGVEGFNCYFPLQSYGFQP

SEQ ID NO: 65 - protein, 4 aa, Artificial Sequence
  SARS-CoV-2 S protein epitope (aa 504-507)
GYQP

SEQ ID NO: 66 - protein, 5 aa, Artificial Sequence
  SARS-CoV-2 S protein epitope (aa 514-518)
SFELL

SEQ ID NO: 67 - protein, 20 aa, Artificial Sequence
  SARS-CoV-2 S protein epitope (aa 551-570)
VLTESNKKFLPFQQFGRDIA

SEQ ID NO: 68 - protein, 18 aa, Artificial Sequence
  SARS-CoV-2 S protein epitope (aa 655-672)
HVNNSYECDIPIGAGICA

SEQ ID NO: 69 - protein, 20 aa, Artificial Sequence
  SARS-CoV-2 S protein epitope (aa 766-785)
ALTGIAVEQDKNTQEVFAQV

SEQ ID NO: 70 - protein, 36 aa, Artificial Sequence
  SARS-CoV-2 S protein epitope (aa 787-822)
QIYKTPPIKDFGGFNFSQILPDPSKPSKRSFIEDLL

SEQ ID NO: 71 - protein, 20 aa, Artificial Sequence
  SARS-CoV-2 S protein epitope (aa 811-830)
KPSKRSFIEDLLFNKVTLAD

SEQ ID NO: 72 - protein, 20 aa, Artificial Sequence
  SARS-CoV-2 S protein epitope (aa 1144-1163)
ELDSFKEELDKYFKNHTSPD

SEQ ID NO: 73 - protein, 12 aa, Artificial Sequence
  SARS-CoV-2 S protein epitope (aa 1147-1158)
SFKEELDKYFKN

SEQ ID NO: 74 - protein, 36 aa, Artificial Sequence
  SARS-CoV-2 S protein epitope (aa 131-166)
CEFQFCNDPFLGVYYHKNNKSWMESEFRVYSSANNC

SEQ ID NO: 75 - protein, 11 aa, Artificial Sequence
  SARS-CoV-2 S protein epitope (aa 291-301)
CALDPLSETKC

SEQ ID NO: 76 - protein, 55 aa, Artificial Sequence
  SARS-CoV-2 S protein epitope (aa 443-495) w flanking cys residues
CSKVGGNYNYLYRLFRKSNLKPFERDISTEIYQAGSTPCNGVEGFNCYFPLQSYC

SEQ ID NO: 77 - protein, 55 aa, Artificial Sequence
  SARS-CoV-2 S protein epitope (aa 443-495_E484K) w flanking cys residues
CSKVGGNYNYLYRLFRKSNLKPFERDISTEIYQAGSTPCNGVKGFNCYFPLQSYC

SEQ ID NO: 78 - protein, 9 aa, Artificial Sequence
  SARS-CoV-2 S protein epitope (aa 480-488_E484K)
CNGVKGFNC

SEQ ID NO: 79 - protein, 53 aa, Artificial Sequence
  SARS-CoV-2 S protein epitope (aa 538-590)
CVNFNFNGLTGTGVLTESNKKFLPFQQFGRDIADTTDAVRDPQTLEILDITPC

SEQ ID NO: 80 - protein, 33 aa, Artificial Sequence
  SARS-CoV-2 S protein epitope (aa 617-649)
CTEVPVAIHADQLTPTWRVYSTGSNVFQTRAGC

SEQ ID NO: 81 - DNA, 30 bp, Artificial Sequence
  Aerosol delivery peptide nucleic acid
tgtgcgaaaagcatgggcgatatcgtgtgt

SEQ ID NO: 82 - DNA, 4179 bp, Artificial Sequence
  MHV type 1 S protein
gaattctgaatgaaatatacaagttatatcttggcttttcagctctgcatcgttttgggttctcttggcatgctgtttgtcgtgtttattctcctaataccctcttgtttagggtatattggtgactttagatgtatccagctcgtgaactcaaacggcaacaacgcttctgcgccaagcattagcattgaaactgtcgatgtttccaaaggccttggtacttattatgttttagatcgagtttatttaaatgccacattattgcttactggttattatcctgtggacggttccaattataggaatctcgcgcttacaggcactaataccctaagccttaattggtataaaccaccctttttatcagagtttaatgatggcatatttgctaaggtaaagaaccttaaagcatctctgcccgctggctcatcggcttatttccctactattataataggcagtggttttggtaacaccgcctatactatagtaatggaaccatataatggtataattatggcatctatttgccagtacaccatttgtcaattaccgtatactgattgtaaacctaatacaggcggtaatagtattataggtttttggcacacagatataaaatcccctgtgtgcattttaaagcgtaatttcacgtttaatgttaatgccgattggctctattttcatttttaccaacagggtggtactttttatgcgtattatgcagatgtagcttctgctactacgtttttatttagtatttatattggcgatgttttaacgcaattctttgtgttgccttttaattgtgaacctgataaggctggtgttatatcaccgcagtattgggtcacacctttagttgagcgccaatatttgtttaattttaaccaaaagggtattattactagtgctgttgattgtgctagtagttataccgctgaaattaaatgcaagactcaaagtatgaatcctagtacgggagtctatgatctcactggttacactgttcaacctgttggtttagtgtaccgaagagttagaaatttgcctgattgtaaaatagaggattggctcaccgctaaaagcgtgccgtctcctctcaattgggaacgtaaaacatttcaaaattgtaactttaacctgagcagtctattaagatttgtccaggctgagtcactctcatgtagtaatatagatgcttccaaagtttatggaatgtgctttggcagcgtatctatagataaatttgcaatacccaataggagacgcgttgacctccaaataggcaattctgggtttttgcagtcttttaattataaaatagattcaagggcgacttcttgccagctttattatagtcttgcacaaaataatgtcaccgttaataaccataacccgtcctcctggaataggcgatatggatttaacgatgtggcaacatttggtagtggtaaacatgacgttgcatatgctgaagagtgttttactgttggtaatgattattgcccatgtgctaaccccagcatagtatcgccatgcacgcaagataaacctaaggctgctaattgtccagtaggtacacgcaatcgagagtgtaaccctctggcgcttggcggtaatttatttaagtgcgactgcacatgtaaccctagcccactaactacctacgaccttcgctgcctccaggctaggagcatgctaggggtaggtgaccattgtgaaggacttggtgttttagaagataaatgtggtggaagcaacgtttgcaattgtactgcagatgcttttgttggctggtctacggatagttgtctatccaaaggccgctgccacattttctcgaatttgttattaaatggcattaatagtggaaccacttgctccactgatttacagttgcctaatactgaagtggttactggcgtttgtgtcaagtatcatctcttcggtattactggtcaaggtgtttttaaggaggttaaagccgactactatcatagctggcagaatctcttatatgatgttaatggcaatctggaaggtttccgcgacatcattaccaataaaacttatactattagaagctgttatagcgggcgagtttcggctgcatatcatcaagatgcacctgaacctgcgctgctatatcgcaatttaaaatgtgattatgtctttaacaacaacatctcccgtgaggagaccccacttaactattttgatagttatttgggctgtgttgttaatgctgataactcaactgaagaagctgttgctgtgtgtgatctacgtatgggtagtggcctttgcgtcaactattcaacgtcacatcgagctcgcaggtccatcagtacgggttataaattaactacttttgaaccatttacagttagcattgtcaatgatagtgttcagtctgtgggtggattatatgagatgcaaataccaatcaattttactataggacaacaccaggagttcattcaaactagagctccaaaggtaactatagattgtgcggcttttgtctgtggtgattacacagcatgccggcagcagttggttgagtatggatcattctgtgataatattaatgccattcttggcgaggttaataacctcatagatactatgcaactgcaggttgctagtgccctgatacaaggtgtcacgctaagttcccgcttggctgatggcattggtggtcagattgatgatattaattttagtcctctgttaggctgtctaggttcagattgtggtgaaggaaccactgctgcactaaagggacggtcggttatagaggatatgctgttcgataaagtcaaactatcagatgttggctttgttgaagcatataataattgcactggtggtcaggaagtcagagacctactatgtgtgcaatcttttaatggcataaaagtgctgcctcctgtattatccgagagtcagatctccggatatacagctggtgctactgcgtctgctatgttcccaccttggtctgcagccgcgggtgtgccattttctttaagtgttcaatatagaattaatggtctgggtgtcactatgaatgttcttagtgaaaaccagaaaatgatagctagtgctttcaacaatgcgattggtgctatacaggagggctttgatgccactaattctgcattagcaaaaattcaatccgttgtgaatgcaaatgctgaagcacttaataatctgttgcaacaattgtccaacagatttggtgcaattagtgcttctttacaggaaattctatcccgccttgatgctcttgaagcgcaggctcagatagaccgtcttataaatggcagattaactgcacttaatgcatatgtttctaagcagctgagtgacatgaccctagttaaggtaagtgccgctcaagctatagagaaagttaatgagtgtgttaaaagccaatcacctaggattaatttctgtggcaatggcaatcatatattgtcattagtccagagtgcgccttatggcttatattttatacacttcagctatgtgcctacatcctttacaacggtaaatgtgagtcctggactttgcatttctggtgatagaggattagcacctaaagctggatattttgttcaagataatggagagtggaagttcactggtagtggttattactaccctgaacccataaatgataaaaacagtgtcgttatgagtagttgtgcagtaaactacacaaaagcgcctgaagttttcttgaacacttcaataccaaatctacccgactttaaggaggagttagataaatggtttaagaatcagacgtccattgcgcctgatttatctctcgatttcgagaaattaaatgttactttcctggacctgaccgatgagatgaacaggattcaggagtcaattaagaagttaaatgagagctacatcaacctcaaggaagttggcacatatgaaatgtatgtgaaatggccttggtacatttggttgctaattggattagctggtgtagctgtttgtgtgttgttattctttatatgctgctgcacaggttgcggctcatgttgttttaagaaatgtggaaattgttgtgatgagtatggaggacaccaggatagtattgtcatccataatatatcctctcacgaggattgaggatcctctagagtcgac

SEQ ID NO: 83 - protein, 1391 aa, Artificial Sequence
  MHV type 1 S protein
EFMKYTSYILAFQLCIVLGSLGMLFVVFILLIPSCLGYIGDFRCIQLVNSNGNNASAPSISIETVDVSKGLGTYYVLDRVYLNATLLLTGYYPVDGSNYRNLALTGTNTLSLNWYKPPFLSEFNDGIFAKVKNLKASLPAGSSAYFPTIIIGSGFGNTAYTIVMEPYNGIIMASICQYTICQLPYTDCKPNTGGNSIIGFWHTDIKSPVCILKRNFTFNVNADWLYFHFYQQGGTFYAYYADVASATTFLFSIYIGDVLTQFFVLPFNCEPDKAGVISPQYWVTPLVERQYLFNFNQKGIITSAVDCASSYTAEIKCKTQSMNPSTGVYDLTGYTVQPVGLVYRRVRNLPDCKIEDWLTAKSVPSPLNWERKTFQNCNFNLSSLLRFVQAESLSCSNIDASKVYGMCFGSVSIDKFAIPNRRRVDLQIGNSGFLQSFNYKIDSRATSCQLYYSLAQNNVTVNNHNPSSWNRRYGFNDVATFGSGKHDVAYAEECFTVGNDYCPCANPSIVSPCTQDKPKAANCPVGTRNRECNPLALGGNLFKCDCTCNPSPLTTYDLRCLQARSMLGVGDHCEGLGVLEDKCGGSNVCNCTADAFVGWSTDSCLSKGRCHIFSNLLLNGINSGTTCSTDLQLPNTEVVTGVCVKYHLFGITGQGVFKEVKADYYHSWQNLLYDVNGNLEGFRDIITNKTYTIRSCYSGRVSAAYHQDAPEPALLYRNLKCDYVFNNNISREETPLNYFDSYLGCVVNADNSTEEAVAVCDLRMGSGLCVNYSTSHRARRSISTGYKLTTFEPFTVSIVNDSVQSVGGLYEMQIPINFTIGQHQEFIQTRAPKVTIDCAAFVCGDYTACRQQLVEYGSFCDNINAILGEVNNLIDTMQLQVASALIQGVTLSSRLADGIGGQIDDINFSPLLGCLGSDCGEGTTAALKGRSVIEDMLFDKVKLSDVGFVEAYNNCTGGQEVRDLLCVQSFNGIKVLPPVLSESQISGYTAGATASAMFPPWSAAAGVPFSLSVQYRINGLGVTMNVLSENQKMIASAFNNAIGAIQEGFDATNSALAKIQSVVNANAEALNNLLQQLSNRFGAISASLQEILSRLDALEAQAQIDRLINGRLTALNAYVSKQLSDMTLVKVSAAQAIEKVNECVKSQSPRINFCGNGNHILSLVQSAPYGLYFIHFSYVPTSFTTVNVSPGLCISGDRGLAPKAGYFVQDNGEWKFTGSGYYYPEPINDKNSVVMSSCAVNYTKAPEVFLNTSIPNLPDFKEELDKWFKNQTSIAPDLSLDFEKLNVTFLDLTDEMNRIQESIKKLNESYINLKEVGTYEMYVKWPWYIWLLIGLAGVAVCVLLFFICCCTGCGSCCFKKCGNCCDEYGGHQDSIVIHNISSHEDGSSRVD

SEQ ID NO: 84 - DNA, 3909 bp, Artificial Sequence
  SARS CoV 2 S Gene
gaattctgaatgaaatatacaagttatatcttggcttttcagctctgcatcgttttgggttctcttggcatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaactctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaggatcctctagagtcgac

SEQ ID NO: 85 - protein, 1301 aa, Artificial Sequence
  SARS CoV 2 S Gene
EFMKYTSYILAFQLCIVLGSLGMFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYTGSSRVD

SEQ ID NO: 86 - protein, 9 aa, Artificial Sequence
  RGD4C-targeting peptide
CDCRGDCFC

SEQ ID NO: 87 - DNA, 93 bp, Artificial Sequence
  Primer
agctttgcctgtccgttcggcgaagtgttcaacgcgacccgcttcgcgagcgtgtatgcgtggaaccgcaaacgcatcagcaactgtcctgca

SEQ ID NO: 88 - DNA, 85 bp, Artificial Sequence
  Primer
ggacagttgctgatgcgtttgcggttccacgcatacacgctcgcgaagcgggtcgcgttgaacacttcgccgaacggacaggcaa

SEQ ID NO: 89 - DNA, 54 bp, Artificial Sequence
  Primer
agctttgcctgttatggcgtgagcccgaccaaactgaacgatctgtgtcctgca

SEQ ID NO: 90 - DNA, 46 bp, Artificial Sequence
  Primer
ggacacagatcgttcagtttggtcgggctcacgccataacaggcaa

SEQ ID NO: 91 - DNA, 42 bp, Artificial Sequence
  Primer
agctttgcctgtaacggcgtggaaggcttcaactgtcctgca

SEQ ID NO: 92 - DNA, 34 bp, Artificial Sequence
  Primer
ggacagttgaagccttccacgccgttacaggcaa

SEQ ID NO: 93 - DNA, 45 bp, Artificial Sequence
  Primer
agctttgcctgtgatatcccgatcggcgcgggcatctgtcctgca

SEQ ID NO: 94 - DNA, 37 bp, Artificial Sequence
  Primer
ggacagatgcccgcgccgatcgggatatcacaggcaa

SEQ ID NO: 95 - DNA, 47 bp, Artificial Sequence
  Primer
agctttgcctgtaccatgtatatctgtggcgatagcaccgaatgtag

SEQ ID NO: 96 - DNA, 37 bp, Artificial Sequence
  Primer
caacctgctgctgcagtatggcagcttctgtcctgca

SEQ ID NO: 97 - DNA, 38 bp, Artificial Sequence
  Primer
ggacagaagatcgccacagatatacatggtacaggcaa

SEQ ID NO: 98 - DNA, 47 bp, Artificial Sequence
  Primer
agctttgcctgtgtgctgggccagagcaaacgcgtggatttctgtcc

SEQ ID NO: 99 - DNA, 21 bp, Artificial Sequence
  Primer
gtggatagcggtttgactcac

SEQ ID NO: 100 - DNA, 26 bp, Artificial Sequence
  Primer
tggtcccagagacatgtatagcatgg

SEQ ID NO: 101 - DNA, 23 bp, Artificial Sequence
  Primer
agggctgttgttcttgtggatcc

SEQ ID NO: 102 - DNA, 26 bp, Artificial Sequence
  Primer
ggacacctagtcagacaaaatgatgc

SEQ ID NO: 103 - DNA, 25 bp, Artificial Sequence
  Primer
agcaagctgataaaccgatacaatt

SEQ ID NO: 104 - DNA, 24 bp, Artificial Sequence
  Primer
ccctcatagttagcgtaacgatct

SEQ ID NO: 105 - protein, 20 aa, Artificial Sequence
  Primer
TGAGGTGGTATCGGCAATGA

SEQ ID NO: 106 - protein, 22 aa, Artificial Sequence
  Primer
GGATGCTGTATTTAGGCCGTTT

SEQ ID NO: 107 - protein, 13 aa, Artificial Sequence
  Probe
TGCCGCGACAGCC

SEQ ID NO: 108 - protein, 29 aa, Artificial Sequence
  Figure 10
TTRTQLPPAYTNSFTRGVYYPDKVFRSSV

SEQ ID NO: 109 - protein, 19 aa, Artificial Sequence
  Figure 10
NNCTFEYVSQPFLMDLEGK

SEQ ID NO: 110 - protein, 81 aa, Artificial Sequence
  Figure 10
HTPINLVRDLPQGFSALEPLVDLPIGINITRFQTLLALHRSYLTPGDSSSGWTAGAAAYYVGYLQPRTFLLKYNENGTITD

SEQ ID NO: 111 - protein, 18 aa, Artificial Sequence
  SARS-CoV-2 S protein epitope (aa 209-226)
PINLVRDLPQGFSALEPL

SEQ ID NO: 112 - protein, 69 aa, Artificial Sequence
  Figure 10
RFPNITNLCPFGEVFNATRFASVYAWNRKRISNCVADYSVLYNSASFSTFKCYGVSPTKLNDLCFTNVY

SEQ ID NO: 113 - protein, 69 aa, Artificial Sequence
  Figure 10
RGDEVRQIAPGQTGKIADYNYKLPDDFTGCVIAWNSNNLDSKVGGNYNYLYRLFRKSNLKPFERDISTE

SEQ ID NO: 114 - protein, 10 aa, Artificial Sequence
  Figure 10
EVRQIAPGQT

SEQ ID NO: 115 - protein, 11 aa, Artificial Sequence
  Figure 10
DYNYKLPDDFT

SEQ ID NO: 116 - protein, 12 aa, Artificial Sequence
  Figure 10
NSNNLDSKVGGN

SEQ ID NO: 117 - protein, 8 aa, Artificial Sequence
  Figure 10
NLKPFERD

SEQ ID NO: 118 - protein, 48 aa, Artificial Sequence
  Figure 10
GSTPCNGVEGFNCYFPLQSYGFQPTNGVGYQPYRVVVLSFELLHAPAT

SEQ ID NO: 119 - protein, 10 aa, Artificial Sequence
  Figure 10
PCNGVEGFNC

SEQ ID NO: 120 - protein, 27 aa, Artificial Sequence
  SARS-CoV-2 S protein epitope (aa 495-521)
YGFQPTNGVGYQPYRVVVLSFELLHAP

SEQ ID NO: 121 - protein, 64 aa, Artificial Sequence
  Figure 10
TGVLTESNKKFLPFQQFGRDIADTTDAVRDPQTLEILDITPCSFGGVSVITPGTNTSNQVAVLY

SEQ ID NO: 122 - protein, 85 aa, Artificial Sequence
  Figure 10
AIHADQLTPTWRVYSTGSNVFQTRAGCLIGAEHVNNSYECDIPIGAGICASYQTQTNSPRRARSVASQSIIAYTMSLGAENSVAY

SEQ ID NO: 123 - protein, 95 aa, Artificial Sequence
  Figure 10
CTMYICGDSTECSNLLLQYGSFCTQLNRALTGIAVEQDKNTQEVFAQVKQIYKTPPIKDFGGFNFSQILPDPSKPSKRSFIEDLLFNKVTLADAG

SEQ ID NO: 124 - protein, 18 aa, Artificial Sequence
  SARS-CoV-2 S protein epitope (aa 769-786)
GIAVEQDKNTQEVFAQVK

SEQ ID NO: 125 - protein, 65 aa, Artificial Sequence
  Figure 10
LLTDEMIAQYTSALLAGTITSGWTFGAGAALQIPFAMQMAYRFNGIGVTQNVLYENQKLIANQFN

SEQ ID NO: 126 - protein, 25 aa, Artificial Sequence
  SARS-CoV-2 S protein epitope (aa 902-926)
MAYRFNGIGVTQNVLYENQKLIANQ

SEQ ID NO: 127 - protein, 24 aa, Artificial Sequence
  Figure 10
QPELDSFKEELDKYFKNHTSPDVD

SEQ ID NO: 128 - DNA, 134 bp, Artificial Sequence
  TGN
cgaattgggatccgagcatcgattgaattctgaatgaaatatacaagttatatcttggcttttcagctctgcatcgttttgggttctcttggctgaggatcctctagagtcgacctgcagaagcttgcctcgat

SEQ ID NO: 129 - DNA, 134 bp, Artificial Sequence
  TGN
gcttaaccctaggctcgtagctaacttaagacttactttatatgttcaatatagaaccgaaaagtcgagacgtagcaaaacccaagagaaccgactcctaggagatctcagctggacgtcttcgaacggagcta

SEQ ID NO: 130 - DNA, 21 bp, Artificial Sequence
  Primer
cgaattgggatccgagcatcg

SEQ ID NO: 131 - DNA, 20 bp, Artificial Sequence
  Primer
atcgaggcaagcttctgcag

SEQ ID NO: 132 - DNA, 21 bp, Artificial Sequence
  Primer
gcttttcagctctgcatcgtt

SEQ ID NO: 133 - DNA, 28 bp, Artificial Sequence
  Primer
gactagtggcaataaaacaagaaaaaca

SEQ ID NO: 134 - DNA, 18 bp, Artificial Sequence
  Primer
tgggttctcttggcatgt

SEQ ID NO: 135 - protein, 66 aa, Artificial Sequence
  SARS-CoV-2 S protein epitope (aa 764-829)
NRALTGIAVEQDKNTQEVFAQVKQIYKTPPIKDFGGFNFSQILPDPSKPSKRSFIEDLLFNKVTLA

SEQ ID NO: 136 - protein, 65 aa, Artificial Sequence
  SARS-CoV-2 S protein epitope (aa 405-469)
DEVRQIAPGQTGKIADYNYKLPDDFTGCVIAWNSNNLDSKVGGNYNYLYRLFRKSNLKPFERDIS

(19) United States
(12) Patent Application Publication
(10) Pub. No.: US 2023/0241203 A1
(43) Pub. Date: Aug. 3, 2023

(54) ENHANCING IMMUNE RESPONSES THROUGH TARGETED ANTIGEN EXPRESSION

(71) Applicants: Rutgers, The State University of New Jersey, New Brunswick, NJ (US); PhageNova Bio, Inc., Summit, NJ (US)

(72) Inventors: Renata Pasqualini, Newark, NJ (US); Wadih Arap, Newark, NJ (US); Steven Libutti, New Brunswick, NJ (US); Christopher Markosian, New Brunswick, NJ (US); Daniela Staquicini, Newark, NJ (US); Fenny Tang, Newark, NJ (US); Tracey Smith, Newark, NJ (US); Virginia Yao, Summit, NJ (US)

(21) Appl. No.: 18/004,517

(22) PCT Filed: Jul. 3, 2021
(86) PCT No.: PCT/US2021/040392

§ 371 Date: Jan. 6, 2023

Related U.S. Application Data
(60) Provisional application No. 63/048,279, filed on Jul. 6, 2020.
(60) Provisional application No. 63/161,136, filed on Mar. 15, 2021.

(51) Int. Cl. A61K 39/215; A61K 45/06; A61P 31/14; C12N 15/86

(52) U.S. Cl. CPC A61K 39/215; A61K 45/06; A61P 31/14; C12N 15/86; A61K 2039/5256

(57) ABSTRACT

The present disclosure includes an immunogenic composition comprising an effective amount of a therapeutic engineered phage and a pharmaceutically acceptable carrier. In certain aspects, the disclosure includes a method of stimulating an immune response in a subject comprising administering to the subject a composition comprising an effective amount of a therapeutic engineered phage. In certain aspects, the disclosure includes a method for treating, ameliorating, and/or preventing a coronavirus infection in a subject comprising administering a composition comprising an effective amount of a therapeutic engineered phage.

### CROSS-REFERENCE TO RELATED APPLICATIONS

The present application claims priority under 35 U.S.C. § 119(e) to U.S. Provisional Patent Application No. 63/048,279, filed Jul. 6, 2020, and U.S. Provisional Patent Application No. 63/161,136, filed Mar. 15, 2021, both of which are hereby incorporated by reference in their entireties herein.

### SEQUENCE LISTING

The contents of the text file named “370602-7034WO1_Sequence_Listing.txt”, which was created on Jul. 3, 2021 and is 74 KB in size, is hereby incorporated by reference in its entirety.

### BACKGROUND OF THE DISCLOSURE

The 2019-2020 coronavirus outbreak is an ongoing pandemic of coronavirus disease 2019 (COVID-19), caused by severe acute respiratory syndrome coronavirus 2 (SARS-CoV-2) first recognized in Wuhan, China. The outbreak was declared a pandemic by the World Health Organization (WHO) on 11 Mar. 2020. As of 28 Jun. 2021, more than 181 million cases of COVID-19 have been reported in over 220 countries and territories, resulting in over 3.93 million deaths. There is an urgent public health need to better understand COVID-19 and SARS-CoV-2, particularly towards the development of medical countermeasures—most importantly and urgently, the development of an effective vaccine.

Ligands capable of homing to vascular beds can be identified following the administration of phage combinatorial peptide libraries. Bacteriophage (phage) are viruses that naturally infect only bacteria. However, they can be engineered to target specific receptors on eukaryotic cells. Studies have demonstrated that phage capsids can be modified with a ligand targeting lymph nodes (LNs), which substantially enhances immune responses against the phage and the antigen it contains. Additionally, the phage genome can be further modified using elements from adeno-associated virus (AAV)—but not the genes encoding the AAV capsid—to make a novel vector termed adeno-associated virus/phage (AAVP). AAVP that selectively home to tissues for antigen presentation, including lung and LN or lymphatic vasculature, can be easily manufactured, expressing transgenes encoding antigens for vaccination. Systemic administration of AAVP is safe in mice, rats, dogs, and non-human primates.

An urgent need exists for effective vaccines against SARS-CoV-2 in order to control the spread of COVID-19. The present disclosure addresses this need.

### SUMMARY OF THE DISCLOSURE

As described herein, the present disclosure relates to immunogenic compositions comprising an effective amount of a therapeutic engineered phage. The present disclosure also includes methods of stimulating an immune response in a subject comprising administering to the subject a composition comprising an effective amount of a therapeutic engineered phage, as well as methods for treating, ameliorating, and/or preventing a coronavirus infection in a subject comprising administering a composition comprising an effective amount of a therapeutic engineered phage.

In one aspect, the disclosure includes an immunogenic composition comprising an effective amount of a therapeutic engineered phage and a pharmaceutically acceptable carrier, wherein the therapeutic engineered phage comprises one or more fusion polypeptides comprising an antigenic polypeptide and a phage coat protein.

In certain embodiments, the therapeutic engineered phage further comprises a fusion polypeptide comprising a tissue-targeting polypeptide and a phage coat protein.

In certain embodiments, the immunogenic composition of any one of claims 1 and 2, wherein the phage coat protein selected from the group comprising pIII protein, pVI protein, pVII protein, pVIII protein, rpVIII protein, and pIX protein.

In certain embodiments, the tissue-targeting polypeptide targets lymph node tissue.

In certain embodiments, the lymph-node tissue targeting polypeptide comprises an amino acid sequence selected from the group comprising SEQ ID NOs: 1-2.

In certain embodiments, the lymph-node tissue targeting polypeptide is encoded by a nucleotide sequence selected from the group comprising SEQ ID NOs: 7-8.

In certain embodiments, the tissue-targeting polypeptide targets lymphatic channel tissue.

In certain embodiments, the lymphatic channel tissue targeting polypeptide comprises an amino acid sequence comprising SEQ ID NO: 3.

In certain embodiments, the lymphatic channel tissue targeting polypeptide is encoded by a nucleotide sequence comprising SEQ ID NO: 9.

In certain embodiments, the tissue-targeting polypeptide targets lung tissue.

In certain embodiments, the lung tissue targeting polypeptide comprises an amino acid sequence selected from the group comprising SEQ ID NO: 4 and 28.

In certain embodiments, the tissue-targeting polypeptide is an integrin-binding domain.

In certain embodiments, the integrin-binding polypeptide comprises an amino acid sequence selected from the group comprising SEQ ID NO: 4, 5, and 86.

In certain embodiments, the integrin-binding polypeptide is encoded by a nucleotide sequence selected from the group comprising SEQ ID NOs: 6 and 81.

In certain embodiments, the tissue-targeting polypeptide is a GRP78-binding domain.

In certain embodiments, the GRP78-binding polypeptide comprises the amino acid sequence selected from the group comprising SEQ ID NOs: 29 and 30.

In certain embodiments, the therapeutic engineered phage further comprises a fusion polypeptide comprising an aerosol delivery polypeptide that targets lung tissue and acts as a transcytosis domain and a phage coat protein.

In certain embodiments, the aerosol delivery polypeptide comprises the amino acid sequence of SEQ ID NO: 4.

In certain embodiments, the aerosol delivery peptide is encoded by a nucleic acid sequence comprising SEQ ID NO: 81

In certain embodiments, the antigenic polypeptide is a viral polypeptide.

In certain embodiments, the viral polypeptide is an epitope derived from a viral protein selected from the group comprising a coronavirus S protein, a coronavirus N protein, a coronavirus M protein, and a coronavirus E protein.

In certain embodiments, the epitope is selected from the group comprising SEQ ID NOs: 10-27, 31-80, 111, 120, 124, 126, 135, and 136.

In certain embodiments, the therapeutic engineered phage is an adeno-associated bacteriophage (AAVP) and further comprises a viral gene.

In certain embodiments, the viral gene is selected from the group comprising a coronavirus S protein, a coronavirus N protein, a coronavirus M protein, and a coronavirus E protein.

In certain embodiments, the viral gene is a coronavirus S protein and encodes an amino acid sequence selected from the group consisting of SEQ ID NOs: 83 and 85.

In certain embodiments, the viral gene is a coronavirus S protein and comprises a nucleotide sequence selected from the group consisting of SEQ ID NOs: 82 and 84.

In another aspect, the disclosure includes a nucleic acid vector comprising the immunogenic composition of any one of claims 1-26.

In certain embodiments, the vector comprises an antigenic polypeptide-pVIII or rpVIII coat protein fusion protein encoding sequence, and tissue-targeting polypeptide-pIII coat protein fusion protein encoding sequence.

In certain embodiments, the vector comprises a tissue-targeting polypeptide-pVIII or rpVIII coat protein fusion protein encoding sequence, and an antigenic polypeptide-containing-pIII coat protein fusion protein encoding sequence.

In certain embodiments, the vector comprises an antigenic polypeptide-pVIII or rpVIII coat protein fusion protein encoding sequence.

In certain embodiments, the vector comprises an antigenic polypeptide-containing-pIII coat protein fusion protein encoding sequence.

In certain embodiments, the vector comprises a 5′ ITR, a CMV promoter, an antigenic polypeptide encoding sequence, a poly-A sequence, a 3′ ITR, and a tissue-targeting polypeptide-pIII coat protein fusion protein-encoding sequence.

In certain embodiments, the vector comprises a 5′ ITR, a CMV promoter, an antigenic polypeptide encoding sequence, a poly-A sequence, a 3′ ITR, a Tac promoter, a tissue-targeting polypeptide-pVIII or rpVIII coat protein fusion protein encoding sequence, and an aerosol delivery polypeptide-pIII coat protein fusion protein encoding sequence.

In certain embodiments, the vector comprises a 5′ ITR, a CMV promoter, an antigenic polypeptide encoding sequence, a poly-A sequence, a 3′ ITR, a Tac promoter, an aerosol-delivery polypeptide-pVIII or rpVIII coat protein fusion protein encoding sequence, and a tissue-targeting polypeptide-pIII coat protein encoding sequence.

In another aspect, the disclosure provides a method of stimulating an immune response in a subject, the method comprising administering to the subject one or more of the immunogenic compositions of any of the above aspects or embodiments or any other aspect or embodiment of the current disclosure.

In certain embodiments, the one or more immunogenic compositions are delivered by a route selected from the group comprising oral route, inhalation route, nasal route, nebulization route, intratracheal route, intravenous injection, intraperitoneal injection, intramuscular injection, subcutaneous injection, and transdermal injection.

In another aspect, the disclosure provides a method for treating, ameliorating, and/or preventing a coronavirus infection in a subject, comprising administering an effective amount of one or more of the immunogenic compositions of any of the above aspects or embodiments or any other aspect or embodiment of the current disclosure.

In certain embodiments, the one or more immunogenic compositions are delivered by a route selected from the group comprising oral route, inhalation route, nasal route, nebulization route, intratracheal, intravenous injection, intraperitoneal injection, intramuscular injection, subcutaneous injection, and transdermal injection.

In certain embodiments, the coronavirus infection is caused by a coronavirus selected from the group comprising SARS-CoV, SARS-CoV-2, HCoV-229E, HCoV-NL63, MERS-CoV, HCoV-OC43, HCoV-HKU1, and murine hepatitis virus, type 1 (MHV-1).

In another aspect, the disclosure provides a method of promoting gene delivery to a virally-infected cell, comprising contacting the cell with a therapeutic engineered phage comprising a fusion protein comprising a ligand-binding polypeptide and a phage coat protein.

In certain embodiments, the phage coat protein selected from the group comprising pIII protein, pVI protein, pVII protein, pVIII protein, rpVIII protein, and pIX protein.

In certain embodiments, the ligand-binding polypeptide selected from the group comprising SEQ ID NOs: 1-5, 28-30, and 86.

In certain embodiments, the therapeutic engineered phage is an adeno-associated virus/phage (AAVP).

In another aspect, the disclosure provides a method of treating, ameliorating, and/or preventing a viral infection in a subject, comprising administering an effective amount of a therapeutic engineered phage comprising a fusion protein comprising a ligand-binding polypeptide and a phage coat protein, thereby treating, ameliorating, and/or preventing the viral infection.

In certain embodiments, the phage coat protein selected from the group comprising pIII protein, pVI protein, pVII protein, pVIII protein, rpVIII protein, and pIX protein.

In certain embodiments, the ligand-binding polypeptide is selected from the group comprising SEQ IDs: 1-5, 28-30, and 86.

In certain embodiments, the ligand-binding polypeptide is a GRP78-binding domain.

In certain embodiments, the GRP78-binding polypeptide comprises the amino acid sequence selected from the group comprising SEQ ID NOs: 29 and 30.

In certain embodiments, the therapeutic engineered phage is an adeno-associated virus/phage (AAVP).

In certain embodiments, the therapeutic engineered phage further comprises an anti-viral agent.

In certain embodiments, the anti-viral agent is selected from the group comprising an anti-viral drug or precursor thereof, an anti-viral polypeptide or precursor thereof, and an anti-viral nucleic acid.

### BRIEF DESCRIPTION OF THE DRAWINGS

The following detailed description of specific embodiments of the disclosure will be better understood when read in conjunction with the appended drawings. For the purpose of illustrating the disclosure, exemplary embodiments are shown in the drawings. It should be understood, however, that the disclosure is not limited to the precise arrangements and instrumentalities of the embodiments shown in the drawings.

FIG. 1 is a graph depicting LN-homing phage eliciting a stronger humoral immune response in comparison to untargeted control phage. Female two-month-old BALB/c mice received intravenous (i.v.) injections of phage displaying PTCAYGWCA (SEQ ID NO: 1), WSCARPLCG (SEQ ID NO: 2), or no peptide (insertless fd-tet phage, negative control) as indicated. Anti-phage antibody serum titers were determined by enzyme-linked immunosorbent assay (ELISA). Shown are the humoral immune response three days after the second vaccination with serum dilutions of 1:500. Data represent the absorbance (A450 nm) of the p-nitrophenyl phosphate substrate (Trepel et al., 2001). Phage-based vaccines use the capsid of the phage particles as the antigen carrier and these particles can be untargeted or targeted to certain organs or cells to improve or enhance antigen presentation.

FIG. 2 is a diagram depicting a map of LN-targeting AAVP designed to generate an immune response against the mouse murine hepatitis virus (MHV) (i.e., murine coronavirus) spike (S) protein. An AAVP construct delivering the gene encoding the MHV S protein is generated using routine molecular biology strategies. To enhance the immune response, LN-targeting peptides are expressed in the pIII minor coat protein to direct AAVP internalization followed by the expression and presentation of the antigen of interest to cells of the immune system. The AAVP-based vaccines use the capsid of the phage particles to target certain organs or cells in order to deliver the gene that encodes the antigen of interest. Cells transduced by AAVP express and present the antigen to improve or enhance the immune response.

FIG. 3 is a series of diagrams depicting non-limiting phage and AAVP constructs acting as vaccines targeting SARS-CoV-2 (COVID-19). The AAVP-based vaccines can use non-limiting peptides for particle delivery and antigenic epitopes.

FIGS. 4A-4C are a series of tables listing certain tissue-targeting, delivery, and antigenic polypeptides of the disclosure that can be expressed either as fusion proteins with phage coat proteins pIII or pVIII (or rpVIII) or as the gene payload of the AAVP vector constructs.

FIGS. 5A-5C are a series of graphs and diagrams illustrating the presence of antibodies against two SARS-CoV-2 epitopes of the present disclosure in human COVID-19 patients.

FIG. 6 is a diagram showing a schematic representation of the phage- and AAVP-based vaccine candidates. The scheme represents the approach used for the conception, design, and application of two strategies of immunization against SARS-CoV-2 S protein using phage particles. Step 1: Structural analysis, selection of structurally-defined epitopes, and cloning steps for the generation of dual-display phage particles and AAVP encoding the full-length S protein. Step 2: Molecular engineering of single- and dual display phage particles, and AAVP S constructs. Step 3: Functional validation and vaccination studies in vivo in mice.

FIGS. 7A-7B illustrate the identification of structural epitopes on SARS-CoV-2 S protein trimer. (FIG. 7A) Six epitopes spanning the SARS-CoV-2 S protein were selected for display on the recombinant phage major coat protein pVIII (rpVIII). Four epitopes are located within the 51 subunit: epitope 1 (SEQ ID NO: 22), epitope 2 (SEQ ID NO: 23), epitope 3 (SEQ ID NO: 24), epitope 4 (SEQ ID NO: 25); and two within the S2 subunit: epitope 5 (SEQ ID NO: 26), and epitope 6 (SEQ ID NO: 27). These epitopes are solvent-exposed in the surface representation of the predominantly closed-state conformation of the S protein trimer (PDB ID: 6ZP0). Only epitope 1 (SEQ ID NO: 22) contains a site for glycosylation (at N343). (FIG. 7B) All of the epitopes maintain a cyclic conformation in the ribbon representation of a S protein protomer; disulfide bridges are present between the flanking cysteine residues of all epitopes except on epitope 2 (SEQ ID NO: 23). The open-state conformation of the S protein trimer with one receptor-binding domain (RBD) erect displays a change in orientation of epitopes 1, 2, and 3, though all remain solvent-exposed (PDB ID: 6ZGG).

FIGS. 8A-8C illustrate the immunogenicity of S epitopes on single-display phage particles. (FIGS. 8A-8B) Five-week-old female Swiss Webster mice were immunized via subcutaneous injection with single-display phage constructs containing each of the six different epitopes expressed on rpVIII or the control insertless phage. Animals received a boost injection three weeks after the first administration. (FIG. 8A) S protein-specific IgG antibodies and (FIG. 8B) phage-specific IgG antibodies were evaluated in sera of mice after two- and five-weeks post-immunization by ELISA (n=3 mice per group). (FIG. 8C) Five-week-old female BALB/c mice were immunized via intratracheal administration with the epitope 4 (SEQ ID NO: 25)/CAKSMGDIVC (SEQ ID NO: 4) dual-display phage particles, epitope 4 (SEQ ID NO: 25) single-display phage particles, or the control insertless phage. Animals received a boost three weeks after the first administration. S protein-specific IgG antibodies were weekly evaluated by ELISA assays (n=10 mice per group). Data represent ±SEM (***P<0.001).

FIGS. 9A-9H illustrate the immunogenicity of RGD4C AAVP SARS-CoV-2 S. Schematic representation of the AAVP-based vaccine candidate. (FIG. 9A) The SARS-CoV-2 S protein gene was excised from the pUC57vector and cloned into the RGD4C targeted AAVP genome. The CoV 2 S protein transgene cassette expression is driven by the cytomegalovirus (CMV) promoter and flanked by AAV ITRs. (FIG. 9B) Schematic representation of the RGD4C AAVP S and control RGD4C AAVP transgene null (AAVP S-null) phage genomes. (FIG. 9C) S protein-specific IgG antibody responses in the sera of mice immunized with RGD4C AAVP S via different routes of administration (n=5 mice per group) were quantified by ELISA in 96-well plates coated with recombinant full-length S protein. (FIG. 9D) S protein-specific IgG antibodies in sera of mice immunized weekly with RGD4C AAVP S or the control RGD4C AAVP S-null (n=12 mice per group) via subcutaneous administration were quantified by ELISA. Graphs show data±SEM (**P<0.01). (FIG. 9E) Tissue-specific expression of the S protein transgene in mice immunized with RGD4C AAVP S five weeks after the first administration was highest in the lymph nodes. Graphs show data±SEM (***P<0.001). (FIG. 9F) Phage-specific IgG antibody response in the sera of mice immunized with RGD4C AAVP S via different routes of administration (n=5 mice per group). Phage-specific IgG antibody response in the sera of mice immunized with RGD4C AAVP S (FIG. 9G) or RGD4C AAVP S-null (FIG. 9H) increased five weeks after initial administration. Phage-specific IgG antibody responses in the sera of treated mice were evaluated by ELISA in 96-well plates coated with 10¹⁰ AAVP particles per well. Tet^{R}, tetracycline resistance gene. Amp^{R}, ampicillin resistance gene. Ori, origin of replication.

FIG. 10 is a diagram of SARS-CoV-2 S protein epitope mapping from peer-reviewed publications. Primary sequence representation of immunogenic regions of S protein spanning S1 and S2 subunits identified by B-cells, T-cells, and antibody screenings of patient sera with COVID-19. The six structurally-selected epitopes displayed on the phage major coat protein rpVIII are highlighted (orange).

FIG. 11 is a diagram illustrating the single- and dual-display phage particles cloning strategy. To generate the single-display phage particles, the f88-4 phage vector was used. This vector contains two genes encoding for the major capsid protein pVIII: the wild-type (pVIII, depicted in grey) and the recombinant (rpVIII, depicted in green). rpVIII contains a foreign DNA insert between the HindIII and PstI cloning site, which allows the cloning of annealed oligonucleotides encoding the S protein epitopes in-frame with the rpVIII gene. For the dual-display phage particles, the f88-4 vector containing the epitope 4 (CDIPIGAGIC-SEQ ID NO:25) and the fUSE55 phage vector are digested with BamHI and XbaI restriction enzymes. The digestion products are then purified and fused according to a standard ligation protocol. The result is a chimeric vector (f88-4/fUSE55). Next, annealed oligonucleotides encoding the CAKSMGDIVC (SEQ ID NO: 4) targeting peptide was cloned within the SfiI restriction sites of the pIII coat protein gene (pIII, depicted in light blue), generating the dual-display phage vector, which contains epitope 4 (SEQ ID NO: 25) on rpVIII and the CAKSMGDIVC (SEQ ID NO: 4) peptide on the pIII. Both, single- or dual-display phage dsDNA were used to transform electrocompetent DH5a E. coli cells. Phage particles were produced in K91 E. coli.

FIGS. 12A-12B illustrate the immunogenicity of S protein epitopes on single- and dual-display phage particles. Five-week-old female BALB/c mice were immunized via the intratracheal (IT) route with 10⁹ transducing units (TU) of epitope 4/CAKSMGDIVC dual-display phage particles, epitope 4 single-display phage particles, or the control insertless phage. (FIG. 12A) S protein-specific IgG antibodies in the sera of mice after 5, 8, and 18 weeks were analyzed by ELISA to evaluate the long-term specific immune response (n=5 mice per group). An additional boost was administered at week 21 and the antibody response was evaluated at week 22. (FIG. 12B) Phage-specific IgG antibody response in the sera of mice immunized with single- and dual-display phage particles (n=10 mice per group). Graphs show data±SEM (*P<0.05, **P<0.01, ***P<0.001).

FIGS. 13A-13G illustrate the immunogenicity of CAKSMGDIVC AAVP S. Schematic representation of the AAVP-based vaccine candidate. (FIG. 13A) The SARS-CoV-2 S protein gene was excised from the pUC57 vector and cloned into the fUSE5 AAVP phage genome. Phosphorylated, annealed oligonucleotides encoding the CAKSMGDIVC targeting peptide were inserted into the SfiI sites in the fUSE5 pIII sequence of fUSE5 AAVP CoV2 S or fUSE5 AAVP transgene null (fUSE5 AAVP S-null) phage genomes. The AAVP transgene cassette is driven by the cytomegalovirus (CMV) promoter and flanked by AAV ITRs. (FIG. 13B) Schematic representation of the CAKSMGDIVC AAVP S and the control CAKSMGDIVC AAVP transgene null phage genome (CAKSMGDIVC AAVP S-null). (FIG. 13C) Schematic representation of the immunization schedule. Five-week-old female BALB/c mice were dosed weekly via the IT route with 10⁹ TU of fd-AAVP-CoV2-S (insertless control AAVP S), CAKSMGDIVC AAVP S or CAKSMGDIVC AAVP S-null phage. (FIG. 13D) The transport of CAKSMGDIVC AAVP S or CAKSMGDIVC AAVP S-null from the lung to the systemic circulation was measured 1 h after IT administration. (FIG. 13E) Tissue-specific expression of the S protein in mice immunized with insertless AAVP S or CAKSMGDIVC AAVP S was observed three weeks after the first immunization. (FIG. 13F) Spike protein-specific IgM antibody response in the sera of treated mice was quantified by ELISA in 96-well plates coated with recombinant full-length S protein. (FIG. 13G) Spike protein-specific IgG antibody response in the sera of treated mice was quantified by ELISA in 96-well plates coated with recombinant full-length S protein.

FIGS. 14A-14B are a table presenting cross-reference analysis of epitope mapping of immunogenic regions of SARS-CoV-2 S protein.

### DETAILED DESCRIPTION

### Definitions

Unless defined otherwise, all technical and scientific terms used herein have the same meaning as commonly understood by one of ordinary skill in the art to which the disclosure pertains. Although any methods and materials similar or equivalent to those described herein can be used in the practice for testing of the present disclosure, selected materials and methods are described herein. In describing and claiming the present disclosure, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic deoxyribonucleic acid (DNA). A skilled artisan will understand that any DNA comprised of nucleotide sequences or a partial nucleotide sequence encoding a protein or peptide that elicits an immune response therefore encodes an “antigen” as that term is used herein. Furthermore, one skilled in the art will understand that an antigen need not be encoded solely by a full-length nucleotide sequence of a gene. It is readily apparent that the present disclosure includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell, or a biological fluid.

As used herein, the term “autologous” is meant to refer to any material derived from the same individual to which it is later to be re-introduced into the individual.

“Allogeneic” refers to any material derived from a different animal of the same species.

“Xenogeneic” refers to any material derived from an animal of a different species.

The term “cleavage” refers to the breakage of covalent bonds, such as in the backbone of a nucleic acid molecule or the hydrolysis of peptide bonds. Cleavage can be initiated by a variety of methods, including, but not limited to, enzymatic or chemical hydrolysis of a phosphodiester bond. Both single-stranded cleavage and double-stranded cleavage are possible. Double-stranded cleavage can occur as a result of two distinct single-stranded cleavage events. DNA cleavage can result in the production of either blunt ends or staggered ends. In certain embodiments, fusion polypeptides may be used for targeting cleaved double-stranded DNA.

As used herein, the term “conservative sequence modifications” is intended to refer to nucleotide or amino acid modifications that do not change the amino acid sequence or significantly affect or alter the binding characteristics of the antibody containing the amino acid sequence, respectively. Amino acid conservative modifications include amino acid substitutions, additions, and deletions. Modifications can be introduced into an antibody of the disclosure by standard techniques known in the art, such as site-directed mutagenesis and polymerase chain reaction (PCR)-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine), and aromatic side chains (e.g., tyrosine, phenylalanine, tryptophan, histidine). Thus, one or more amino acid residues within the complementarity-determining regions (CDRs) of an antibody can be replaced with other amino acid residues from the same side chain family and the altered antibody can be tested for the ability to bind antigens using the functional assays described herein.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The term “downregulation” as used herein refers to the decrease or elimination of gene expression of one or more genes.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to, anti-tumor activity as determined by any means suitable in the art.

“Encoding” refers to the inherent property of specific sequences of nucleotides in a polynucleotide, such as a gene, a complementary DNA (cDNA), or a messenger ribonucleic acid (mRNA), to serve as templates for synthesis of other polymers and macromolecules in biological processes having either a defined sequence of nucleotides [i.e., ribosomal RNA (rRNA), transfer RNA (tRNA) and messenger RNA (mRNA)] or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein, “endogenous” refers to any material from or produced inside an organism, cell, tissue or system.

As used herein, the term “exogenous” refers to any material introduced from or produced outside an organism, cell, tissue or system.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes), and viruses (e.g., Sendai viruses, lentiviruses, retroviruses, adenoviruses, and AAV) that incorporate the recombinant polynucleotide.

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

The term “immunoglobulin” or “Ig,” as used herein is defined as a class of proteins, which function as antibodies. Antibodies expressed by B cells are sometimes referred to as the B cell receptor (BCR) or antigen receptor. The five members included in this class of proteins are IgA, IgG, IgM, IgD, and IgE. IgA is the primary antibody that is present in body secretions, such as saliva, tears, breast milk, gastrointestinal secretions, and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is the immunoglobulin that has no known antibody function but may serve as an antigen receptor. IgE is the immunoglobulin that mediates immediate hypersensitivity by causing the release of mediators from mast cells and basophils upon exposure to an allergen.

The term “immune response” as used herein is defined as a cellular and humoral response to an antigen that occurs when lymphocytes and antigen-presenting cells identify antigenic molecules as foreign and induce the formation of antibodies and/or activate lymphocytes to remove the antigen. The immune response can be mediated by acellular and cellular components. The acellular components include physical barriers and signaling molecules such as cytokines. The cellular response is mediated by both innate immune cells such as macrophages, neutrophils, dendritic cells, and adaptive immune cells such as lymphocytes (T and B). Both cellular and humoral aspects contribute to the production of antibodies, clearance of the antigen, and the development of immunological memory.

When “an immunologically effective amount,” “an autoimmune disease-inhibiting effective amount,” or “therapeutic amount” is indicated, the precise amount of the compositions of the present disclosure to be administered can be determined by a physician or researcher with consideration of individual differences in age, weight, tumor size, extent of infection or metastasis, and condition of the patient (subject).

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “knockdown” as used herein refers to a decrease in gene expression of one or more genes.

The term “knockout” as used herein refers to the ablation of gene expression of one or more genes.

The term “adeno-associated virus” or “AAV” as used herein refers to small, nonpathogenic, single-stranded DNA viruses of the genus Dependoparvovirus. Their ability to readily infect both dividing and quiescent human cells and tissues with minimal immune responses and no obvious pathogenicity has led to the use of AAV as vectors for gene therapy and vaccines.

The term “coronavirus” as used herein refers to a member of Coronaviridae, a family of enveloped, positive-sense single-strand RNA viruses. Coronaviruses can cause disease in birds and mammals. One of the most well-studied coronaviruses is the murine coronavirus MHV, which causes epidemic infections in laboratory animals. In humans, coronaviruses typically cause respiratory infections that range in severity from the common cold to more lethal diseases such as SARS, Middle East Respiratory Syndrome (MERS), and COVID-19.

The term “phage” or “bacteriophage” as used herein refer to viruses that evolved to infect and replicate within prokaryotic or archaeal cells. Bacteriophages can comprise either RNA or DNA genomes and can have protein capsid structures of varying complexity. In humans, phage therapy has been used as an alternative to antibiotics for the treatment of bacterial infection. Phage particles can also be engineered to infect eukaryotic cells, and as such make attractive vectors for gene therapy in that they can be easily expanded to vast quantities in bacterial cultures and their novel structure means pre-existing immunity in humans is relatively low.

The term “limited toxicity” as used herein, refers to the peptides, polynucleotides, cells and/or antibodies of the disclosure manifesting a lack of substantially negative biological effects, anti-tumor effects, or substantially negative physiological symptoms toward a healthy cell, non-tumor cell, non-diseased cell, non-target cell or population of such cells either in vitro or in vivo.

By the term “modified” as used herein, is meant a changed state or structure of a molecule or cell of the disclosure. Molecules may be modified in many ways, including chemically, structurally, and functionally. Cells may be modified through the introduction of nucleic acids.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably a human.

In the context of the present disclosure, the following abbreviations for the commonly occurring nucleic acid bases when discussing nucleic acid sequences are used. “A” or “a” refers to adenosine, “C” or “c” refers to cytosine, “G” or “g” refers to guanosine, “T” or “t” refers to thymidine, and “U” or “u” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some versions contain an intron(s).

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

“Parenteral” administration of an immunogenic composition includes, e.g., subcutaneous (s.c.), intravenous (i.v.), intramuscular (i.m.), or intrasternal injection, or infusion techniques.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides, and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific.

In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The term “subject” is intended to include living organisms in which an immune response can be elicited (e.g., mammals). A “subject” or “patient,” as used therein, may be a human or non-human mammal. Non-human mammals include, for example, livestock and pets, such as ovine, bovine, porcine, canine, feline and murine. Preferably, the subject is human.

As used herein, a “substantially purified” cell is a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are cultured in vitro. In other embodiments, the cells are not cultured in vitro.

A “target site” or “target sequence” refers to a genomic nucleic acid sequence that defines a portion of a nucleic acid to which a binding molecule may specifically bind under conditions sufficient for binding to occur. “Target site” or “target sequence” can also refer to a protein sequence that defines a portion of a protein to which a binding molecule or polypeptide may specifically bind under conditions sufficient for binding to occur.

The term “therapeutic” as used herein means a treatment and/or prophylaxis. A therapeutic effect is obtained by suppression, remission, or eradication of a disease state.

The term “transfected” or “transformed” or “transduced” as used herein refers to a process by which exogenous nucleic acid is transferred or introduced into the host cell. In the case of a targeted phage, the exogenous nucleic acid is initiated by a ligand-receptor binding event followed by a receptor-mediated internalization event. A “transfected” or “transformed” or “transduced” cell is one which has been transfected, transformed or transduced with exogenous nucleic acid. The cell includes the primary subject cell and its progeny.

To “treat” a disease as the term is used herein, means to reduce the frequency or severity of at least one sign or symptom of a disease or disorder experienced by a subject.

The phrase “under transcriptional control” or “operatively linked” as used herein means that the promoter is in the correct location and orientation in relation to a polynucleotide to control the initiation of transcription by RNA polymerase and expression of the polynucleotide.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, Sendai viral vectors, adenoviral vectors, adeno-associated viral vectors, retroviral vectors, lentiviral vectors, AAVP, and the like.

Ranges: Throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### DESCRIPTION

In one aspect, the current disclosure relates the finding that filamentous phage vectors can be modified to express one or more polypeptides that direct the phage particles to bind ligand proteins expressed by specific tissues and induce beneficial immune responses against specific antigens. These engineered phage particles can express the antigenic polypeptides in the presence or absence of targeted polypeptides. Both tissue-targeting and antigenic polypeptides are expressed as fusion proteins with one or more phage coat proteins, such that they are displayed on the outer surface of the phage particle. In this way, these modifications enhance the utility of the engineered phage vectors to be used as therapeutic and prophylactic vaccines. In certain embodiments, the phage coat protein is wildtype. In certain embodiments, the phage coat protein is recombinant.

In some embodiments, the tissue-targeting polypeptides direct the phage particles to lung tissue, LN tissue, and/or lymphatic vessel tissue. In some embodiments, the tissue-targeting polypeptides bind to cell-surface integrins. In some embodiments, the tissue-targeting polypeptide binds to GRP78. In some embodiments, the tissue-targeting polypeptide binds to PPP2R1A. In some embodiments, the tissue-targeting polypeptide binds to C16-ceramide.

In some embodiments, the therapeutic engineered phage further comprises an aerosol delivery polypeptide that targets lung tissue and acts as a transcytosis domain. In some embodiments, the antigenic polypeptides are epitopes derived from coronavirus proteins. In a preferred embodiment, the antigenic polypeptides are epitopes derived from the S protein of SARS-CoV-2.

In some embodiments, the therapeutic engineered phage particles further comprise genomic elements of AAV and phage, and act as vectors for polynucleotide payloads, which allow the particles to transduce mammalian cells and express exogenous polypeptides. In some embodiments, the AAVP vector acts as a vaccine by delivering a viral gene to antigen-presenting cells, which then induces a productive immune response against the protein. In some embodiments, the viral gene encodes a coronavirus S protein. In some embodiments, the S protein is derived from SARS-CoV-2 or MHV. In some embodiments, the AAVP vector is targeted to cell-surface GRP78 expressed by cells undergoing stress conditions including viral-infection. In some embodiments, the AAVP vector is targeted to the LN or lymphatic channels. In some embodiments, the AAVP vector is targeted to the lungs. In some embodiments, AAVP then delivers an antiviral agent that inhibits viral function. This inhibition can be achieved by various methods, including but not limited to: delivering chemotherapeutic drugs or prodrugs; expressing polypeptides toxic to the function of the virus by either indirect or direct inhibition of viral proteases and structural proteins; and/or inducing cell death by expression of various pro-apoptotic polypeptides.

Also provided is a method of stimulating an immune response in a subject comprising administering to the subject a composition comprising an effective amount of the therapeutic engineered phage particles of the disclosure. The present disclosure also provides a method for treating, ameliorating, and/or preventing a coronavirus infection in a subject comprising administering a composition comprising an effective amount of the therapeutic engineered phage particles of the disclosure.

### Filamentous Phage Display

In certain embodiments, the disclosure includes phage particles displaying polypeptides used to target the particles to certain tissues and act as epitopes for stimulating specific immune responses. These polypeptides can be displayed on the surface of the phage particles by being fused to phage coat proteins in a manner similar to that used in phage display. Phage display is a method using bacteriophage particles as scaffolds to display recombinant libraries of peptides or proteins and provide a vehicle to recover and amplify the peptides or proteins that bind to putative ligand molecules or antigens. In some embodiments of the current disclosure, polypeptides fused to phage coat proteins are used as antigens to stimulate immune responses and to direct the phage particles to specific tissues. In some embodiments, the coat proteins of the phage particles can comprise either an antigenic polypeptide or a tissue-targeting polypeptide. In some embodiments, the phage particles comprise coat proteins that express both a tissue-targeting polypeptide and an antigenic polypeptide.

Phage that display proteins or peptides as a fusion with a phage coat protein are designed to contain appropriate coding regions of the coat proteins. A variety of bacteriophage and coat proteins may be used. Examples include, without limitation, M13 gene III, gene VI, gene VII, gene VIII, and gene IX; fd minor coat protein pIII (Saggio et al., Gene 152:35, 1995); f88-4 major recombinant pVIII (rpVIII) coat protein (Scott and Smith, Science 249 (4967):386, 1990); lambda D protein (Sternberg & Hoess, Proc. Natl. Acad. Sci. USA 92:1609, 1995; Mikawa et al., J. Mol. Biol. 262:21, 1996); lambda phage tail protein pV (Maruyama et al., Proc. Natl. Acad. Sci. USA 91:8273, 1994; U.S. Pat. No. 5,627,024); fr coat protein (WO 96/11947; DD 292928; DD 286817; DD 300652); X29 tail protein gp9 (Lee, Viol. 69:5018, 1995); MS2 coat to protein; T4 small outer capsid protein (Ren et al., Protein Sci. 5:1833, 1996), T4 nonessential capsid scaffold protein IPIII (Hong and Black, Virology 194:481, 1993), or T4 lengthened fibritin protein gene (Efimov, Virus Genes 10:173, 1995); PRD-1 gene III; Q33 capsid protein (as long as dimerization is not interfered with); and P22 tail spike protein (Carbonell & Villaverde, Gene 176:225, 1996). Techniques for inserting a foreign coding sequence into a phage gene sequence are well known to one of ordinary skill in the art (see e.g., Sambrook et al., Molecular Cloning: A Laboratory Approach, Cold Spring Harbor Press, N Y, 1989; Ausubel et al., Current Protocols in Molecular Biology, Greene Publishing Co., NY, 1995).

Compared to other bacteriophage, filamentous phage in general are attractive for use as display scaffolds for polypeptides, with M13 being particularly amenable for a number of reasons: (1) the 3D structure of the virion is known; (2) the processing of the coat protein is well understood; (3) the genome is small enough to allow relatively large payload proteins; (4) the sequence of the genome is known; (5) the virion is physically resistant to shear, heat, cold, urea, guanidinium Cl, low pH, and high salt; (6) it is easily cultured and stored, with no unusual or expensive media requirements for the infected cells; (7) it has a high burst size with each infected cell yielding 100 to 1,000 M13 progeny after infection; and (8) it is easily harvested and concentrated.

The filamentous phage include: M13, fl, fd, Ifl, Ike, Xf, Pf1, f88-4 or “Type 88” and Pf3. (Webster (1996) Chapter 1, Biology of the Filamentous Bacteriophage, in Kay et al., eds. (1996) Phage Display of Peptides and Proteins). The entire life cycle of the filamentous phage M13, a common cloning and sequencing vector, is well understood in the art. The genetic structure (the complete sequence, the identity and function of the ten genes, and the order of transcription and location of the promoters) of M13 is well known as is the physical structure of the virion. Because the genome is small (6423 bp), cassette mutagenesis is practical on M13, as is single-stranded oligonucleotide directed mutagenesis. The M13 genome is expandable and M13 does not lyse cells. Because the M13 genome is extruded through the membrane and coated by a large number of identical protein molecules, it can be used as a cloning vector. Thus, payload genes can be engineered into M13 and they can be carried along in a stable manner.

The fd pIII minor coat protein is a non-limiting outer surface protein utilized in many, phage display systems because it is present in only a few copies and because its location and orientation in the virion are known. For example, only three to five copies of protein pIII are displayed on the ends of each phage particle. The limited number of pIII proteins present make peptides fused to them present at a low valency per particle, which is desirable in situations where a limited number of displayed peptides per phage particle is desired; for example, in the selection of high-affinity interactions. In certain embodiments, tissue-targeting and antigenic polypeptides can be fused to the pIII protein such that they are displayed on the surface of the phage particle.

Each fd bacteriophage expresses about 2,700 copies of the pVIII major coat protein which are arranged in stacked helical arrays of five proteins. The f88 vectors (including f88-4; GenBank Accession AF218363) are Type 88 vectors, in which the phage genome bears two genes VIII, encoding two different types of pVIII molecule. One pVIII is recombinant (i.e., bears a foreign DNA insert) and the other wild-type. The recombinant gene VIII is synthetic and differs in nucleotide sequence from the wild-type gene (though it largely encodes the wild-type amino acid sequence). The f88 virion is a mosaic, its coat being composed of both wild-type and recombinant (r) pVIII subunits; the latter typically comprise about 150 of the 3900 subunits. This allows hybrid pVIII proteins with quite large foreign peptides to be displayed on the virion surface, even though the hybrid protein by itself cannot support phage assembly. As a result, peptides expressed in fusion with rpVIII proteins are present at a relatively high valency of around 200 copies per phage particle. The increased avidity effect of high valency pVIII display permits selection of low-affinity ligands or is advantageous when relatively large amounts of the fused peptide are needed. In certain embodiments of the current disclosure, tissue-targeting and antigenic polypeptides can be fused with the pVIII or rpVIII protein of the therapeutic engineered phage particles. In some embodiments, the therapeutic engineered phage particles can express both pIII and pVIII or rpVIII fusion coat proteins such that antigenic peptides can be targeted to specific tissues in order to stimulate optimal immune responses.

### Bacteriophage as Vaccines

Bacteriophage possess a number of qualities that make them ideal candidates for use as vaccine platforms. Phage particles are highly stable under harsh conditions and can be easily and inexpensively produced at large-scale quantities using well-established manufacturing techniques. Phage particles also possess potent adjuvant capabilities, in that they are readily recognized by the mammalian immune system without being pathogenic due to their inability to infect eukaryotic cells. While the use of phage as medical treatments originally focused on their inherent anti-bacterial function, current uses harness their potent immunogenic potential. In certain embodiments of the current disclosure, phage particles are engineered to express specific antigenic polypeptides in fusion with phage coat proteins. In this way, immune recognition and priming against the phage particles also stimulate immune responses against the fusion polypeptide, thus providing beneficial immune responses against specific epitopes. In some embodiments, these immune responses are directed against epitopes derived from the coronavirus proteins, thus acting as an immunotherapy against coronavirus infection or providing protective immunity against a potential coronavirus infection. In some embodiments, the phage particles further comprise elements of adeno-associated virus (AAV) genome and are AAVP hybrid vectors capable of delivering the viral gene or fragments thereof to target cells that will express and present glycosylated viral antigens to the immune system.

### Adeno-Associated Virus/Phage (AAVP)

AAV are relatively small, non-enveloped viruses with a ˜4 kb genome that is flanked by inverted terminal repeats (ITRs). The genome contains two open reading frames, one of which provides proteins necessary for replication and the other provides components required for construction of the viral capsid. Wild-type AAV is typically found in the presence of adenovirus as the adenoviruses provide helper proteins that are essential for packaging of the AAV genome into virions. Therefore, AAV production piggy-backs on co-infection with adenovirus and relies on three key elements: the ITR-flanked genome, the open-reading frames, and adeno-helper genes. Due to their non-pathogenic ability to readily infect human cells, AAV is well-studied as a vector for gene delivery. AAV may be readily obtained and their use as vectors for gene delivery has been described in, for example, Muzyczka, 1992; U.S. Pat. No. 4,797,368, and PCT Publication WO 91/18088. Construction of AAV vectors is described in a number of publications, including Lebkowski et al., 1988; Tratschin et al., 1985; Hermonat and Muzyczka, 1984

AAVP are hybrid vectors combining elements of AAV type 2 and filamentous bacteriophage genomes (Nature Protocols 2, 523-531(2007); Cell 125, 385-398 (2006)). Namely, AAVP gene expression is under the control of a eukaryotic transgene cassette flanked by internal terminal repeats (ITRs) of AAV2 and inserted in an intergenomic region of the bacteriophage genome. In this way, the vector combines the specificity of phage vectors with the characteristics of transgene expression by AAV, yielding a virus that can reproduce specifically and easily in prokaryotic cells, efficiently bind to receptors via a ligand-receptor interaction mediated by the targeting peptide ligand, internalize into mammalian cells via a subsequent receptor-mediated event and express the transgene similar to AAV. Hence, the AAVP vector possesses favorable characteristics of mammalian and prokaryotic viruses and does not suffer from the disadvantages that those individual vectors normally carry.

The advantages of phage or AAVP particles as antigen carrier vaccines are listed: (1) they are highly stable under harsh environmental conditions and their large-scale production is extremely cost-effective if compared to traditional methods used for vaccine production; (2) several studies have demonstrated that phage-based vaccines do not induce detectable toxic side effects and because phage and AAVP do not replicate inside eukaryotic cells, their use is generally considered safe when compared to other classic viral-based vaccination strategies; (3) unlike conventional peptide-based vaccines that may often become inactivated due to minimal temperature excursions (˜1° C.), phage or AAVP vaccines have no cumbersome and expensive requirements for keeping a stringent so-called “cold-chain” during field applications, particularly in the developing world.

In certain embodiments of the current disclosure, the therapeutic engineered phage particles of the disclosure further comprise genomic elements of AAV and are AAVP hybrid vectors. In certain embodiments, the AAVP of the disclosure comprise fusion coat proteins comprising tissue-targeting polypeptide that direct the AAVP to cells expressing specific target ligands. In certain embodiments, the AAVP of the disclosure are gene delivery vectors that express exogenous proteins in target cells. For instance, in certain embodiments, the exogenous protein is a viral protein that is expressed in tissue-resident antigen-presenting cells, thereby stimulating an adaptive immune response against the exogenous protein. In certain embodiments, the viral protein is an S protein from a coronavirus, and the AAVP of the disclosure acts as a vaccine or immunotherapy. In some preferred embodiments, the S protein is derived from SAPS-CoV-2. In some preferred embodiments, the S protein is derived from MHV.

### Tissue-Targeting Ligands

The cells of the body express unique surface proteins or molecules which account for the extensive morphological and functional diversity of the tissues which they comprise. These unique molecules or groups of molecules can be targeted by specific ligands to deliver agents such as drug or imaging molecules to specific tissues in both in vitro and in vivo experimental models, as well as directly in human patients. These tissue-targeting ligands can be specific for normal tissue, as well as diseases or disorders including but not limited to cancer, viral infections, bacterial infections, or otherwise normal cells involved in disease states.

Tissue-targeting polypeptides can take a number of forms, including but not limited to antibodies or antigen-binding fragments thereof, and ligands of receptors expressed by the target cells or fragments thereof. Recent studies have identified that peptides of about 7-15 amino acids in length can bind to cell surface ligands with relatively high affinity and specificity. Given their relatively short length, these ligand-binding polypeptides can be easily attached to molecules or proteins by chemical conjugation or expressed as fusion proteins by genetic engineering.

In some embodiments, the therapeutic engineered phage of the disclosure expresses a fusion polypeptide that binds to receptor proteins largely expressed in lung tissue. A non-limiting example of such lung-targeting polypeptide is the sequence CGSPGWVRC (SEQ ID NO: 28), which binds to sphingolipid C16-ceramide and is abundantly expressed on human and murine lung vascular endothelial cells. In contrast to many ceramide-inducing stimuli, this peptide does not activate apoptosis, which is advantageous for lung targeting without inducing toxicity to the lung tissue. In other embodiments, the tissue-targeting ligand targets αv integrins and comprises the amino acid sequence ACDCRGDCFCG (SEQ ID NO: 5). The αv integrins are cell surface receptors overexpressed on both tumor and certain endothelial cells (Arap et al., 1998; Hood et al., 2002). Recent studies have demonstrated the use of such a targeting peptide to direct imaging and therapy molecules to tumor tissue (Hajitou et al., 2006).

In some embodiments of the disclosure, the ligand-binding polypeptide comprises the sequence CGLTFKSLC (SEQ ID NO: 3) and targets the PPP2R1A protein, a molecule which is abundantly expressed in lymphatic channels and in association with metastatic melanoma (Christianson et al., 2015).

In some embodiments of the disclosure, the ligand-binding polypeptides target LN tissue and can comprise the amino acid sequences PTCAYGWCA (SEQ ID NO: 1) and WSCARPLCG (SEQ ID NO: 2). The use of such LN-targeting peptides is useful to direct antigens to lymphoid tissue, which are primary sites of adaptive immune function including antibody production and priming of antigen-specific T cell responses and are particularly useful for phage particles acting as vaccines and immune adjuvants (Trepel et al., 2001).

In some embodiments of the current disclosure, the ligand-binding polypeptide targets the GRP78 protein. GRP78, also known as heat shock protein family A (HSP70) member 5 or HSPA5 is a chaperone molecule that is normally expressed intracellularly in the endoplasmic reticulum (ER) where it plays a key role in directing protein folding in the ER lumen. Cells that are under stress, including by viral infection or through transformation into cancer, may express significant amounts of GRP78 on their cell surface. In this way, targeting of GRP78 can be utilized to target molecules specifically to stressed or cancerous cells while avoiding normal tissues that do not express cell surface GRP78.

In some embodiments of the disclosure, a GRP78-targeting polypeptide of the amino acid sequence CSNTRVAPC (SEQ ID NO: 29) and WIFPWIQL (SEQ ID NO: 30) are used to target the therapeutic engineered phage particles of the disclosure to cells undergoing stress conditions. In some embodiments, the stress condition is a viral infection (Ferrara et al., 2016).

These phage particles can further comprise anti-viral agents that can be used to block or inhibit viral function and ultimately treat the infection. Examples of such anti-viral agents can include but are not limited to chemotherapy drugs, or prodrug and/or active metabolites thereof, proteins that directly inhibit viral enzymes or structural proteins, and pro-apoptotic polypeptides that induce the selective ablation of viral-infected cells.

### Pharmaceutical Compositions

Pharmaceutical compositions of the present disclosure may comprise the therapeutic engineered phage particles as described herein, in combination with one or more pharmaceutically or physiologically acceptable carriers, diluents or excipients. Such compositions may comprise buffers such as neutral buffered saline, phosphate-buffered saline (PBS) and the like; carbohydrates such as glucose, mannose, sucrose or dextran, mannitol; proteins; polypeptides or amino acids such as glycine; antioxidants; chelating agents such as EDTA or glutathione; adjuvants (e.g., aluminum hydroxide); and preservatives. Compositions of the present disclosure are preferably formulated for a number of administration routes including oral, inhalation, nasal, nebulization, intravenous injection, intramuscular injection, subcutaneous injection, and/or transdermal injection.

Pharmaceutical compositions of the present disclosure may be administered in a manner appropriate to the disease to be treated (or prevented). The quantity and frequency of administration will be determined by such factors as the condition of the patient, the type and severity of the patient's disease, and the type and functional nature of the patient's immune response to the phage particles, although appropriate dosages may be determined by clinical trials.

The therapeutic engineered phage particles of the disclosure can be administered in dosages and routes and at times to be determined in appropriate pre-clinical and clinical experimentation and trials. Phage particle compositions may be administered multiple times at dosages within these ranges. Administration of the phage particles of the disclosure may be combined with other methods useful to treat the desired disease or condition as determined by those of skill in the art.

It can generally be stated that a pharmaceutical composition comprising the engineered phage particles described herein may be administered at a dosage of at least about 10⁷, about 10⁸, about 10⁹, about 10¹⁰, about 10¹¹, about 10¹², or about 10¹³ transducing units (TU) or phage particles/kg, including all integer values within those ranges. Dosage size can be adjusted according to the weight, age, and stage of the disease of the subject being treated. Phage particles may also be administered multiple times at these dosages. The phage particles can be administered by using infusion techniques that are commonly known in the art of immunotherapy or vaccinology. The optimal dosage and treatment regime for a particular patient can readily be determined by one skilled in the art of medicine by monitoring the patient for signs of disease and adjusting the treatment accordingly.

The administration of the phage compositions of the disclosure may be carried out in any convenient manner known to those of skill in the art. The phage of the present disclosure may be administered to a subject by aerosol inhalation, injection, ingestion, transfusion, implantation or transplantation. The compositions described herein may be administered to a patient trans-arterially, subcutaneously, intranasally, intradermally, intratumorally, intranodally, intramedullary, intramuscularly, intravenously, or intraperitoneally. In other instances, the phage of the disclosure are injected directly into a site of inflammation in the subject, a local disease site in the subject, a LN, an organ, a tumor, and the like.

It should be understood that the method and compositions that would be useful in the present disclosure are not limited to the particular formulations set forth in the examples. The following examples are put forth so as to provide those of ordinary skill in the art with a complete disclosure and description of how to make and use the phage particles, expansion and culture methods, and therapeutic methods of the disclosure, and are not intended to limit the scope of what the inventors regard as their disclosure.

The practice of the present disclosure employs, unless otherwise indicated, conventional techniques of molecular biology (including recombinant techniques), microbiology, cell biology, biochemistry, and immunology, which are well within the purview of the skilled artisan. Such techniques are explained fully in the literature, such as, “Molecular Cloning: A Laboratory Manual”, fourth edition (Sambrook, 2012); “Oligonucleotide Synthesis” (Gait, 1984); “Culture of Animal Cells” (Freshney, 2010); “Methods in Enzymology” “Handbook of Experimental Immunology” (Weir, 1997); “Gene Transfer Vectors for Mammalian Cells” (Miller and Calos, 1987); “Short Protocols in Molecular Biology” (Ausubel, 2002); “Polymerase Chain Reaction: Principles, Applications and Troubleshooting”, (Babar, 2011); “Current Protocols in Immunology” (Coligan, 2002). These techniques are applicable to the production of the polynucleotides and polypeptides of the disclosure, and, as such, may be considered in making and practicing the disclosure. Particularly useful techniques for particular embodiments will be discussed in the sections that follow.

### EXPERIMENTAL EXAMPLES

The disclosure is now described with reference to the following Examples. These Examples are provided for the purpose of illustration only, and the disclosure is not limited to these Examples, but rather encompasses all variations that are evident as a result of the teachings provided herein.

The materials and methods employed in these experiments are now described.

Animals. Four-to-six-week-old Swiss Webster and BALB/c mice were purchased from The Jackson Laboratory (Sacramento, Calif.) and were housed in specific pathogen and opportunist free (SOPF) rooms with controlled temperature (20±2° C.), humidity (50±10%), light cycle (light, 7:00-19:00; dark, 19:00-7:00), and access to food and water ad libitum at the research animal facilities of the Rutgers Cancer Institute of New Jersey. Littermates were randomly assigned to experimental groups. The Institutional Animal Care and Use Committee (IACUC) from the Rutgers Cancer Institute of New Jersey approved all animal experiments.

Structural Analysis of S Protein for Epitope Selection. The structure of SARS-CoV-2 S protein (PDB IDs: 6VXX, 6VYB) was analyzed using UCSF Chimera software for selection of epitopes to display on rpVIII. Though epitope 3 (SEQ ID NO: 24) was not resolved in these early structures, the flanking cysteine residues of this region were predicted to form a disulfide bridge. This has been confirmed experimentally with since-determined structures (e.g., PDB IDs: 6ZP0, 6ZGG).

Cloning and activity in vitro of the AAVP constructs: DNA encoding the SARS-CoV-2 S glycoprotein, MHV type 1 S protein, or control genes are synthesized and incorporated into the backbone of the AAVP construct displaying CDCRGDCFC, CAKSMGDIVC, LN- or lymphatic channel-targeted peptides using well-established methods. LN-targeting AAVP constructs carrying control genes or modifications for protein secretion or transmembrane docking are also tested. The same strategy is applied to the SARS-CoV-2 S glycoprotein and MHV-1 S protein. AAVP genomic DNA are amplified in electrocompetent E. coli cells (MC1061, Invitrogen) and purified using plasmid purification kits (Qiagen or Invitrogen) following the manufacturer's instructions. All constructs are fully sequenced for phage production and larger-scale production. Each AAVP construct is purified from bacterial culture supernatants and quantified by both infecting K91/Kan E. coli and by qPCR.

Generation of S Protein Epitopes Single-display Phage Particles. To generate single-display or dual-display phage constructs, the M13-derived vector f88-4 containing a recombinant gene VIII (GenBank Accession Number: AF218363.1) was transformed into MC1061F⁻E. coli. Single colonies were selected on Luria-Bertani (LB) agar plates with tetracycline (40 μg/mL) and streptomycin (50 μg/mL) and cultured overnight (O.N.). Each plasmid DNA was first isolated by standard plasmid purification kit (Qiagen). Next, annealed oligonucleotides encoding for each of the six selected epitopes:

epitope 1:
fwd
(SEQ ID NO: 87)
5’ AGCTTTGCCTGTCCGTTCGGCGAAGTGTTCAACGCGACCCGCTTCG
CGAGCGTGTATGCGTGGAACCGCAAACGCATCAGCAACTGTCCTGCA
3’,
rev
(SEQ ID NO: 88)
5’ GGACAGTTGCTGATGCGTTTGCGGTTCCACGCATACACGCTCGCGA
AGCGGGTCGCGTTGAACACTTCGCCGAACGGACAGGCAA 3’;
epitope 2:
fwd
(SEQ ID NO: 89)
5’ AGCTTTGCCTGTTATGGCGTGAGCCCGACCAAACTGAACGATCTGT
GTCCTGCA 3’,
rev
(SEQ ID NO: 90)
5’ GGACACAGATCGTTCAGTTTGGTCGGGCTCACGCCATAACAGGCAA
3’;
epitope 3:
(SEQ ID NO: 91)
5’ AGCTTTGCCTGTAACGGCGTGGAAGGCTTCAACTGTCCTGCA 3’,
rev
(SEQ ID NO: 92)
5’ GGACAGTTGAAGCCTTCCACGCCGTTACAGGCAA 3’;
epitope 4:
fwd
(SEQ ID NO: 93)
5’ AGCTTTGCCTGTGATATCCCGATCGGCGCGGGCATCTGTCCTGCA
3’,
rev
(SEQ ID NO: 94)
5’ GGACAGATGCCCGCGCCGATCGGGATATCACAGGCAA 3’;
epitope 5:
fwd
(SEQ ID NO: 95)
5’ AGCTTTGCCTGTACCATGTATATCTGTGGCGATAGCACCGAATGTA
G 3’,
rev
(SEQ ID NO: 96)
5’ CAACCTGCTGCTGCAGTATGGCAGCTTCTGTCCTGCA 3’;
epitope 6:
fwd
(SEQ ID NO: 97)
5’ GGACAGAAGATCGCCACAGATATACATGGTACAGGCAA 3’,
rev
(SEQ ID NO: 98)
5’ AGCTTTGCCTGTGTGCTGGGCCAGAGCAAACGCGTGGATTTCTGTC
C 3’

were mixed at equimolar ratio and annealed using a thermocycler (93° C. for 3 minutes, 80° C. for 20 minutes, 75° C. for 20 minutes, 70° C. for 20 minutes, 65° C. for 20 minutes, 40° C. for 60 minutes). Annealed double stranded oligonucleotides were cloned into the f88-4 plasmid previously digested with HindIII and PstI restriction endonucleases as described. Restriction enzyme-digested and sequence-verified individual clones were electroporated into DH5α E. coli electrocompetent cells. Phage particles were produced in K91 E. coli cultured in LB media containing 1 mM IPTG, tetracycline (40 μg/mL), and kanamycin (100 μg/mL), and were purified by the polyethylene glycol (PEG)-NaCl method. The titration of single-display phage particles was carried out by infection of host bacterial cells K91 E. coli for colony counting and represented as transducing units (TU/μL).

Generation of Dual-display Phage Particles. To produce phage particles simultaneously displaying epitope 4 (SEQ ID NO: 25) and the lung transport peptide CAKSMGDIVC (SEQ ID NO: 4), the single-display phage constructs (described above) and fUSE55 genome were fused to create a chimeric vector. The f88-4 vector-derived DNA fragment containing the rpVIII gene was inserted in the fUSE55 phage vector, by double digesting both vectors with XbaI and BamHI restriction enzymes at 37° C., 4 h. After incubation, DNA fragments were loaded onto an agarose gel (0.8%, wt/vol). Under an ultraviolet transilluminator, the DNA fragment of 3,925 bp of fUSE55 and the 5,402-bp DNA fragment of the epitope 4 of f88-4 vector (in-frame with the rpVIII gene) were excised. Fifty nanograms (ng) of fUSE55 DNA fragments were ligated to 68.8 ng of f88-4 DNA fragment with T4 DNA ligase (1U) in a final volume of 20 μL, O.N. at 16° C. for 16 h. An aliquot of the ligation reaction was transformed into DH5α E. coli electrocompetent cells and inoculated on LB agar plate containing 40 μg/mL of tetracycline. Positive clones were selected by sequencing analysis and the plasmid containing the chimeric vector was purified with a QIAprep Spin Miniprep kit (Qiagen). Next, oligonucleotide encoding for the CAKSMGDIVC (SEQ ID NO: 4) targeting motif was cloned within the SfiI restriction sites of the pIII coat protein gene (pIII), generating the dual-display phage vector, which contains epitope 4 (SEQ ID NO: 25) on the rpVIII and the CAKSMGDIVC (SEQ ID NO: 4) motif on the pIII. The titration of dual-display phage particles was carried out by infection of host bacterial cells K91 E. coli.

Genetic Engineering and Production of RGD4C-AAVP S and RGD4C-AAVP S-null Particles. The 3.821 kb SARS-CoV-2 spike glycoprotein (S) coding sequence (Genbank Accession number NC_045512.2) was synthesized at GeneWiz (South Plainfield, N.J.) with modifications to simplify subcloning into the RGD4C-AAVP-TNF genome. The single EcoRI restriction site of the SARS-CoV-2 S gene (SEQ. ID NO: 84) at 1371 bp was deleted by replacing the thymidine nucleotide at position 1380 to cytosine, which did not change the translated asparagine residue at position 460 (SEQ. ID NO: 85). The 69-nucleotide sequence of the human interferon leader sequence and the 19-nucleotide sequence of the poly A region in RGD4C-AAVP-TNF were added to the 5′ or 3′ ends, respectively, to the modified synthetic CoV-2 S gene to produce a 3.909 kb modified CoV-2 S gene that was subcloned into the EcoRI and SalI restriction sites of pUC57/AmpR at GeneWiz. The first EcoRI restriction site at 829 bp within the AgeI and KasI restriction sites in RGD4C-AAVP-TNF was deleted in two steps to mutate a thymidine to cytosine nucleotide at position 833, which changed an asparagine residue to an aspartate at amino acid residue 200, using the Q5 site-directed mutagenesis kit (New England Biolabs, Ipswich, Mass.) by following the manufacturer's protocol. Positive clones were verified by EcoRI restriction mapping, confirmed with overlapping sense and antisense primers by Sanger sequencing (SeqStudio, Thermo Fisher Scientific) using the BigDye Terminator v.3.1 Cycle Sequencing and XTerminator Purification kits (Applied Biosystems, Thermo Fisher Scientific) and analyzed using SnapGene software (GSL Biotech, San Diego, Calif.). Positive RGD4C-AAVP-TNFΔEcoRI829/MC1061 F⁻ colonies containing a unique EcoRI site at 10,191 kb were identified by EcoRI restriction mapping of dsDNA purified from 50 mL overnight animal-free LB Broth, pH 7.4 cultures containing 100 μg/mL streptomycin (VWR, Radnor, Pa.) and 40 μg/mL tetracycline (Gold Biotechnology, Inc., St. Louis, Mo.) using the PureLink HiPure Plasmid MidiPrep kit (Invitrogen, Thermo Fisher Scientific). Sequences near the AgeI and KasI cloning sites were confirmed by Sanger sequencing in both directions as described above. The modified, synthetic CoV-2 S gene was ligated into the EcoRI/SalI sites of dephosphorylated, gel-purified, RGD4C-AAVPΔEcoRI829 digested with EcoR1-HF and SalI-HF (New England Biolabs) using a 1:3 vector:insert ratio and a vector mass of 15 ng. Ligation products were transformed into animal-free, electrocompetent MC1061 F⁻E. coli and plated onto animal-free LB agar containing 100 μg/mL streptomycin and 40 μg/mL tetracycline (MilliporeSigma). Positive clones were verified by restriction mapping and confirmed by Sanger sequencing of purified dsDNA as described above.

The 84 bp transgene null (TGN) sequence containing the upstream AAVP human interferon leader sequence ending with a stop codon (bold, underlined) was synthesized at Integrated DNA Technologies (San Diego, Calif.) as a 134 bp dsDNA G-block with an EcoRI site is at the 5′ end and a SalI site is at the 3′ end.

| (SEQ ID NO: 129) | |
|---|---|
| 1 | CGAATTGGGA TCCGAGCATC GATTGAATTC TGAATGAAAT ATACAAGTTA |
| 1 | GCTTAACCCT AGGCTCGTAG CTAACTTAAG ACTTACTTTA TATGTTCAAT |
| 51 | TATCTTGGCT TTTCAGCTCT GCATCGTTTT GGGTTCTCTT GGCTGAGGAT |
| 51 | ATAGAACCGA AAAGTCGAGA CGTAGCAAAA CCCAAGAGAA CCGACTCCTA |
| 101 | CCTCTAGAGT CGACCTGCAG AAGCTTGCCT CGAT |
| 101 | GGAGATCTCA GCTGGACGTC TTCGAACGGA GCTA |

The TGN sequence was PCR amplified using the following primers: FOR primer: 5′ GTGGATAGCGGTTTGACTCAC (SEQ ID NO: 99) 3′ and REV primer 5′ GGACACCTAGTCAGACAAAATGATGC (SEQ ID NO: 102) 3′, digested with EcoRI-HF and SalI-HF (New England Biolabs), purified (Invitrogen PureLink Quick Gel Extraction and PCR Purification Combo Kit, Thermo Fisher Scientific), subcloned into dephosphorylated, gel-purified RGD4C-AAVP-TNFΔEcoRI830 digested with the same restriction enzymes, transformed into animal-free electrocompetent MC1061 F⁻ and plated onto LB agar Lennox plates containing 100 μg/mL streptomycin and 40 μg/mL tetracycline. Single transformed colonies were screened by colony PCR, using the following primers: FOR primer: 5′ GTGGATAGCGGTTTGACTCAC (SEQ ID NO: 99) 3′ and REV primer 5′ GGACACCTAGTCAGACAAAATGATGC (SEQ ID NO: 102) 3′ to identify the presence of the TGN sequence as a 963 bp PCR product in a 1.2% E-gel (Invitrogen, Thermo Fisher Scientific). Putative positive RGD4C-AAVP-transgene null phage (AAVP S-null) were verified in putative positive clones by restriction mapping and Sanger sequencing in both directions as described above.

Genetic Engineering of insertless-AAVP S, CAKSMGDIVC-, CGLTFKSLC- and PTCAYGWCA-AAVP S and CAKSMGDIVC-, CGLTFKSLC- and PTCAYGWCA-AAVP S-null Phage Particles. The 3.139 kb BsrGI-PacI fragment of fUSE5 dsDNA containing a 23 bp stuffer region with 2 SfiI restriction sites in lieu of a targeting peptide sequence in the pIII gene was subcloned into the BsrGI-HF (New England Biolabs) and PacI (Thermo Fisher Sci) sites of dephosphorylated, gel-purified RGD4C-AAVP-TNFΔEcoRI829 to produce fUSE5-AAVP-TNFΔEcoRI829. The sequence of the 3.139 kb BsrGI-PacI fragment was confirmed by Sanger sequencing as described above.

The 3.116 kb BsrGI-PacI fragment from fd-Tet (Genbank Accession Number AF217317) was subcloned into fUSE5-AAVP-TNFΔEcoRI829 to create the untargeted fd-AAVP-TNFΔEcoRI829. Ligation products were transformed into animal-free, electrocompetent MC1061 F⁻ E. coli and screened by colony PCR. Double-stranded DNA from positive clones were purified, verified by restriction mapping and confirmed by Sanger sequencing as described above. Replacement of the tnfα gene with the modified synthetic SARS-CoV-2 S gene proceeded as described above. dsDNA from putative fd-AAVP-CoV2 S/MC1061 F⁻ clones were verified by colony PCR, restriction mapping and confirmed by Sanger sequencing as described above.

The TNF gene was replaced with the modified, synthetic CoV-2 S gene in fUSE5-AAVP-TNFΔEcoRI829 and fd-AAVP-TNFΔEcoRI829 as described above to produce fUSE5-AAVP-CoV2 S or fd-AAVP-S, respectively. Transformed fUSE5-AAVP-CoV2 S/MC1061 F⁻ colonies were screened by colony PCR using sets of primers within and outside the SARS-CoV-2 S gene and amplified with DreamTaq Polymerase (Thermo Fisher Scientific). The 5′ region was amplified using the forward primer 5′ GTGGATAGCGGTTTGACTCAC 3′ (SEQ ID NO: 99) and reverse primer 5′ TGGTCCCAGAGACATGTATAGCATGG 3′ (SEQ ID NO: 100) to produce a 1.058 kb PCR product. The 3′ region was amplified using the forward primer 5′ AGGGCTGTTGTTCTTGTGGATCC 3′ (SEQ ID NO: 101) and reverse primer 5′ GGACACCTAGTCAGACAAAATGATGC 3′ (SEQ ID NO: 102) to produce a 268 bp PCR product. Putative positive fUSE5-AAVP-CoV2 S/MC1061 F⁻ or insertless AAVP S/MC1061 F⁻ colonies were identified by gel electrophoresis of the PCR products. Purified dsDNA from putative positive clones were verified by restriction mapping and confirmed by Sanger sequencing as described above using either the colony PCR products or purified dsDNA as the template.

Synthetic sense and antisense oligonucleotides (MilliporeSigma) encoding the targeting peptide sequences CAKSMGDIVC (SEQ ID NO: 4), CGLTFKSLC (SEQ ID NO: 3), or PTCAYGWCA (SEQ ID NO: 1) were reconstituted to 100 μM with nuclease-free water (Life Technologies, Thermo Fisher Scientific), and 5′ hydroxyl groups were phosphorylated with T4 polynucleotide kinase (New England Biolabs). Phosphorylated oligonucleotide pairs were denatured at 95° C. for 3 minutes and annealed in decreasing increments of 5° C. for 20 minutes each, starting at 80° C. to 65° C., incubated at 40° C. for 60 minutes and held at 4° C. (Applied Biosystems Proflex PCR System, Thermo Fisher Scientific). Annealed oligonucleotides were ligated into the fUSE5 stuffer region of fUSE5-AAVP-CoV2 S or fUSE5-AAVP-S-null digested with SfiI (New England Biolabs) overnight at 16° C. using a 20:1 insert:vector ratio and transformed into animal-free, electrocompetent MC1061 F⁻ bacteria to produce CAKSMGDIVC(SEQ ID NO:4) AAVP S, CAKSMGDIVC(SEQ ID NO:4) AAVP S-null, CGLTFKSLS(SEQ ID NO:3) AAVP S, CGLTFKSLC(SEQ ID NO:3) AAVP S-null, PTCAYGWCA (SEQ ID NO:1) AAVP S or PTCAYGWCA (SEQ ID NO:1) S-null. Transformants were screened by colony PCR and amplified by DreamTaq polymerase using the fUSE5 forward (5′ AGCAAGCTGATAAACCGATACAATT 3′ (SEQ ID NO: 103) and reverse (5′ CCCTCATAGTTAGCGTAACGATCT 3′ (SEQ ID NO: 104) primers. The predicted 274 bp PCR products were electrophoresed in 4% E-Gels (Thermo Fisher Scientific) for size comparison against positive (RGD4C-AAVP-TNF) and negative (fUSE5) controls. dsDNA from putative positive clones were verified by restriction mapping and confirmed by Sanger sequencing as described above.

Production of targeted AAVP S or AAVP S-null phage. A single transformed RGD4C-AAVP-CoV2 S/MC1061 F⁻ or RGD4C-AAVP-CoV2 SNull/MC1061 F⁻, CAKSMGDIVC-AAVP-CoV2 S/MC1061 F⁻ or CAKSMGDIVC-AAVP-CoV2 SNull/MC1061 F⁻, CGLTFKLSC-AAVP-CoV2 S/MC1061 F⁻ or CGLTFKLSC-AAVP-CoV2 SNull/MC1061 F⁻, or insertless AAVP-CoV2 S/MC1061 F⁻ colony was used to inoculate 10 mL of animal-free LB Broth, pH 7.4 containing 100 μg/mL streptomycin and 40 μg/mL tetracycline and grown to mid-log phase at 37° C. and 250 rpm in the dark. One milliliter of the mid-log phase pre-culture was used to inoculate 750 mL of animal-free LB Broth, pH 7.4 containing 100 μg/mL streptomycin and 40 μg/mL tetracycline in a sterile, 2 L shaker baffle flask for phage amplification at 30° C., 250 rpm for 20 hours in the dark. Phage were precipitated in sterile PEG 8000/3.3 M NaCl (15% v/v) and the final phage pellet was resuspended in 1 mL sterile phosphate-buffered saline, pH 7.4, centrifuged to remove residual bacterial debris and filtered sterilized through a 0.2 μm syringe filter. Phage titers (transducing units (TU)/μL) were determined by infecting K91 E. coli grown in animal-free terrific broth with 1×10⁷, 1×10⁸ or 1×10⁹ diluted phage, plating the infected bacteria onto animal-free LB agar plates containing 100 μg/mL kanamycin sulfate and 40 μg/mL tetracycline and counting bacterial colonies the following day. Phage genome copy number/μL was quantified by TaqMan qPCR (QuantStudio™ 7, Thermo Fisher Scientific) using the qPhage forward 5′ TGAGGTGGTATCGGCAATGA 3′ (SEQ ID NO: 105) and reverse 5′ GGATGCTGTATTTAGGCCGTTT 3′ (SEQ ID NO: 106) primers (Invitrogen) and the TaqMan probe: 5′ VIC-TGCCGCGACAGCC-MGBNFQ (SEQ ID NO: 107) (Applied Biosystems, Thermo Fisher Scientific) using the TaqMan Fast Advanced Master Mix (Applied Biosystems) to produce an 85 bp amplicon.

Mouse immunization and protective immunity: Mice are immunized intravenously, subcutaneously, intratracheally or intranasally with the LN-targeting AAVP encoding MHV type 1 S protein or control genes following published procedures. Two weeks after immunization, mice are examined for the presence of protective immunity against MHV type 1 virus. For the protective immunity experiments, C57BL/6J A/J mice are used (6-8-week-old, 20 females and 20 males) utilizing published protocols showing that this mouse strain develops severe lung disease when infected with, type MHV-1 (De Albuquerque, et al. (2006) J Virol 80: 10382-10394). At the time of challenge, mice receive intranasal inoculation of 5×10³ PFU MHV type 1 suspended in Dulbecco's modified Eagle medium (day 0). Mice are monitored daily for symptoms of disease, ruffled fur, tremors, and lack of activity. Mice are sacrificed on days 0, 2, 7, 14, and 21 post infection (eight animals per time point). At each time point, blood is collected via cardiac puncture and stored at −80° C. Lung infection is monitored by homogenizing the right lung in PBS and determining infectious dose (ID50) by a standard plaque assay in L2 cells. The left lung is fixed with 10% formalin for histology and immunohistochemistry. Sections of the lung are scored for presence of edema in lung air spaces and perivascular inflammation. Viral antigen is detected by immunohistochemistry, utilizing rabbit anti-nucleocapsid antibody and appropriate detection reagents.

Swiss Webster or BALB/C mice were randomized in groups of 3 to 12 animals. Group size was calculated based on statistical considerations to yield sufficient statistical significance. The animals were inoculated with 10⁹ TU phage or AAVP constructs intraperitoneally, intravenously, intratracheally or subcutaneously. For subcutaneous injections, 10⁹ TU of phage or AAVP particles were administered with 100 μL on the front and hind limbs, and behind the neck (˜20 μL per site). For intratracheal vaccination, 10⁹ TU of single-, dual-display phage particles or negative control insertless phage particles were administered in two serial doses in 50 μL of PBS with a MicroSprayer® Aerosolizer coupled to a high-pressure syringe (Penn-Century) and a small animal laryngoscope (Penn-Century). The devices were used to administer air-free liquid aerosol directly into the trachea of animals deeply anesthetized with 1% isoflurane. For the tail vein blood collections, mice were locally anesthetized with a topic solution. At day 0, blood samples were collected for the baseline, followed by consecutive blood collection every 1-2 weeks post-immunization. Endotoxin removal was performed for each purified phage or AAVP preparation prior to administration of each dose, regardless of the route of administration. Purified phage or AAVP containing endotoxin were treated with 10% Triton X-114 in endotoxin-free water on ice for 10 min, warmed to 37° C. degrees for 10 min followed by separation of the Triton X-114 phase by centrifugation at 14,000 rpm for 1 min. The upper aqueous phase containing phage was withdrawn into a sterile microcentrifuge tube. This process was repeated from 3-5 times. The levels of endotoxin were measured using the Limulus Amebocyte Lysate (LAL) Kinetic-QCL kit from Lonza. Phage or AAVP preparations containing endotoxin levels <0.05 EU/mL were used in this study.

Antibody response to AAVP produced antigens. To measure serum titers for tissue-targeting AAVP construct antibodies, 96-well plates are coated with a solution of SARS-CoV-2 S glycoprotein or MHV type 1 S protein or the control protein (10 ug/mL; Sigma, St. Louis, Mo.) as reported (D. E. Portal, et al. (2019) Cancer Gene Ther). Following blocking with PBS plus fetal calf serum (FCS), a dilution series of mouse sera before and after treatment are added to the wells and incubated for one hour; the plates are washed and serum antibodies are visualized using a peroxidase-labeled anti-mouse IgG heavy and light chain second reagent (Caltag, South San Francisco, Calif.) and o-phenylenediamine substrate. Titers are read at 492 nm as the reciprocal serum dilution yielding 50% maximum absorbance in the assay.

Serological Analysis. To detect the presence of S protein-specific antibodies or phage-specific IgG antibodies, ELISA assays were performed in 96-microwell plates coated with 150 ng/well of SARS-CoV-2 Spike (aa 16-1213) His-tagged recombinant protein (ThermoFisher) and 1010 phage or AAVP particles/50 μL of phosphate-buffered saline (PBS) ON at 4° C. (Nunc MaxiSorp flat bottom, ThermoFisher Scientific). Coated plates were blocked with PBS containing 5% low-fat milk and 1% bovine serum albumin (BSA) for 1 h at 37° C. Two-fold serial dilutions (starting at 1:32) or 1:50 fixed dilution of sera in blocking buffer were added to separate the wells and incubated for 1-2 h at 37° C. Following three washes with PBS and PBS containing 0.1% of Tween 20, bound antibodies were detected with an anti-mouse IgG HRP-conjugated (Jackson ImmunoResearch) at optical density (OD) at 450 nm. Commercially available polyclonal IgG anti-Spike protein antibody (Thermo Fisher, MA5-35949) or anti-fd bacteriophage antibody (Sigma Aldrich) served as positive controls.

RNA Isolation and Quantitative Real-Time PCR: To measure tissue-specific expression of the S protein transgene in mice immunized with AAVP S, total RNA from mice tissues were obtained with the RNeasy Mini Kit (Qiagen). First-strand cDNA synthesis was carried out with the ImProm-II Reverse Transcription System (Promega). Quantitative real-time PCR analysis was performed in a QuantStudio 5 Real-Time PCR System (Applied Biosystems). Primers and TaqMan probes were as follows: fwd 5′ GCTTTTCAGCTCTGCATCGTT (SEQ ID NO: 132) 3′ and rev 5′ GACTAGTGGCAATAAAACAAGAAAAACA (SEQ ID NO: 133) 3, customized AAVP S 6FAM 5′ TGGGTTCTCTTGGCATGT (SEQ ID NO: 134) 3′ NFQ, Mm04277571_s1 for 18S, and Mm99999915_g1 for Gapdh. The gene expression ratio was normalized to that of 18S.

TABLE 1
Phage component sequences

| SEQ | | |
|---|---|---|
| ID NO: | Name: | Sequence: |
| 1. | LN-targeting peptide | PTCAYGWCA |
| 2. | LN-targeting peptide | WSCARPLCG |
| 3. | Lymphatic channel- | CGLTFKSLC |
| | targeting peptide | |
| 4. | Aerosol delivery peptide | CAKSMGDIVC |
| 5. | RGD4C-targeting peptide | ACDCRGDCFCG |
| 6. | RGD4C-targeting peptide | gcgtgtgattgtaggggggattgtttttgtggc |
| | nucleic acid | |
| 7. | LN-targeting peptide | ccgacctgtgcgtatggctggtgtgcg |
| | nucleic acid | |
| 8. | LN-targeting peptide | tggagctgtgcgcgcccgctgtgtggc |
| | nucleic acid | |
| 9. | Lymphatic channel- | tgtggcctgaccttcaaaagcctgtgt |
| | targeting peptide nucleic | |
| | acid | |
| 10. | SARS-CoV-2 S protein | CNGTNQGGGYGGGYQGYGC |
| | optimized epitope | |
| 11. | SARS-CoV-2 S protein | CNTNQGGYGGYQYC |
| | optimized epitope | |
| 12. | SARS-CoV-2 S protein | CYQYGGGGYGGQNTGGGGNC |
| | optimized epitope | |
| 13. | SARS-CoV-2 S protein | CYQYGGYGQNTGNC |
| | optimized epitope | |
| 14. | SARS-CoV-2 S protein | CGGLQYGGGGYGQTNGGGNC |
| | optimized epitope | |
| 15. | SARS-CoV-2 S protein | CLQYGGYQTNGGNC |
| | optimized epitope | |
| 16. | SARS-CoV-2 S protein | CNGGGNTQGYGYSQYGGGTC |
| | optimized epitope | |
| 17. | SARS-CoV-2 S protein | CNGNTQYYSQYGTC |
| | optimized epitope | |
| 18. | SARS-CoV-2 S protein | CHTNSWGGGTNNCC |
| | optimized epitope | |
| 19. | SARS-CoV-2 S protein | CYSNNSGGTGGNEQC |
| | optimized epitope | |
| 20. | SARS-CoV-2 S protein | CYGTQNGTGGGYGGTQNGTC |
| | optimized epitope | |
| 21. | SARS-CoV-2 S protein | CNNSQGGGGGNNSQGGGGGC |
| | optimized epitope | |
| 22. | SARS-CoV-2 S protein | CPFGEVFNATRFASVYAWNRKRISNC |
| | epitope (aa 336-361) | |
| 23. | SARS-CoV-2 S protein | CYGVSPTKLNDLC |
| | epitope (aa 379-391) | |
| 24. | SARS-CoV-2 S protein | CNGVEGFNC |
| | epitope (aa 480-488) | |
| 25. | SARS-CoV-2 S protein | CDIPIGAGIC |
| | epitope (aa 662-671) | |
| 26. | SARS-CoV-2 S protein | CTMYICGDSTECSNLLLQYGSFC |
| | epitope (aa 738-760) | |
| 27. | SARS-CoV-2 S protein | CVLGQSKRVDFC |
| | epitope (aa 1032-1043) | |
| 28. | Lung-targeting peptide | CGSPGWVRC |
| 29. | GRP78-targeting peptide | CSNTRVAPC |
| 30. | GRP78-targeting peptide | WIFPWIQL |
| 31. | SARS-CoV-2 Spike | TESNKKFLPFQQFGRDIA |
| | protein epitope | |
| | (aa 553-570) | |
| 32. | SARS-CoV-2 Spike | PSKPSKRSFIEDLLFNKV |
| | protein epitope | |
| | (aa 809-826) | |
| 33. | SARS-CoV-2 Spike | YNSASFSTFKCYGVSPTK |
| | protein epitope | |
| | (aa 369-386) | |
| 34. | SARS-CoV-2 S protein | KVCEFQFCNDPFLGVYYHKNNKSWMESEFRVYS |
| | epitope (aa 129-161) | |
| 35. | SARS-CoV-2 S protein | GDSSSGWTAGAAAYYVGYLQPRTFLLKYNENGT |
| | epitope (aa 252-284) | |
| 36. | SARS-CoV-2 S protein | GEVFNATRFASVYAWNRKRISNCVADYSVLYNS |
| | epitope (aa 339-371) | |
| 37. | SARS-CoV-2 S protein | KPFERDISTEIYQAGSTPCNGVEGFNCYFPLQS |
| | epitope (aa 462-494) | |
| 38. | SARS-CoV-2 S protein | SYQTQTNSPRRARSVASQSIIAYTMSLGAENSV |
| | epitope (aa 673-705) | |
| 39. | SARS-CoV-2 S protein | CTFEYVSQPFLMDLE |
| | epitope (aa 166-180) | |
| 40. | SARS-CoV-2 S protein | NLLLQYGSFCTQLNR |
| | epitope (aa 751-765) | |
| 41. | SARS-CoV-2 S protein | TDEMIAQYTSALLAG |
| | epitope (aa 866-880) | |
| 42. | SARS-CoV-2 S protein | NFSQILPDPSKPSKR |
| | epitope (aa 801-815) | |
| 43. | SARS-CoV-2 S protein | TESNKKFLPFQQ |
| | epitope (aa 553-564) | |
| 44. | SARS-CoV-2 S protein | RDPQTLEILDIT |
| | epitope (aa 577-588) | |
| 45. | SARS-CoV-2 S protein | VSVITPGTNTSNQVAV |
| | epitope (aa 595-612) | |
| 46. | SARS-CoV-2 S protein | HADQLTPTWRVYSTGSNV |
| | epitope (aa 625-642) | |
| 47. | SARS-CoV-2 S protein | ECDIPIGAGICASYQTQTNSPRRA |
| | epitope (aa 661-684) | |
| 48. | SARS-CoV-2 S protein | FKEELDKYFKNH |
| | epitope (aa 1148-1159) | |
| 49. | SARS-CoV-2 S protein | RTQLPPAYTNSFTRGVYYPDKVFRS |
| | epitope (aa 21-45) | |
| 50. | SARS-CoV-2 S protein | SALEPLVDLPIGINITRFQTLLALH |
| | epitope (aa 221-245) | |
| 51. | SARS-CoV-2 S protein | GAAAYYVGYLQPRTFLLKYNENGTI |
| | epitope (aa 261-285) | |
| 52. | SARS-CoV-2 S protein | PNITNLCPFGEVFNATRFAS |
| | epitope (aa 330-349) | |
| 53. | SARS-CoV-2 S protein | NSASFSTFKCYGVSPTKLNDLCFTN |
| | epitope (aa 370-394) | |
| 54. | SARS-CoV-2 S protein | STFKCYGVSPTKLNDLCFTN |
| | epitope (aa 375-394) | |
| 55. | SARS-CoV-2 S protein | EVRQIAPGQTGK |
| | epitope (aa 406-417) | |
| 56. | SARS-CoV-2 S protein | QTGKIADYNYKLPD |
| | epitope (aa 414-427) | |
| 57. | SARS-CoV-2 S protein | IADYNYKLPDDFT |
| | epitope (aa 418-430) | |
| 58. | SARS-CoV-2 S protein | KLPDD |
| | epitope (aa 424-428) | |
| 59. | SARS-CoV-2 S protein | SNNLDSKVGGN |
| | epitope (aa 438-448) | |
| 60. | SARS-CoV-2 S protein | NYLYRLFRKSNLKPFERDIS |
| | epitope (aa 450-469) | |
| 61. | SARS-CoV-2 S protein | RLFRKSNLKP |
| | epitope (aa 454-463) | |
| 62. | SARS-CoV-2 S protein | SNLKPFERD |
| | epitope (aa 459-467) | |
| 63. | SARS-CoV-2 S protein | TPCNGVEGFNC |
| | epitope (aa 478-488) | |
| 64. | SARS-CoV-2 S protein | CNGVEGFNCYFPLQSYGFQP |
| | epitope (aa 480-499) | |
| 65. | SARS-CoV-2 S protein | GYQP |
| | epitope (aa 504-507) | |
| 66. | SARS-CoV-2 S protein | SFELL |
| | epitope (aa 514-518) | |
| 67. | SARS-CoV-2 S protein | VLTESNKKFLPFQQFGRDIA |
| | epitope (aa 551-570) | |
| 68. | SARS-CoV-2 S protein | HVNNSYECDIPIGAGICA |
| | epitope (aa 655-672) | |
| 69. | SARS-CoV-2 S protein | ALTGIAVEQDKNTQEVFAQV |
| | epitope (aa 766-785) | |
| 70. | SARS-CoV-2 S protein | QIYKTPPIKDFGGFNFSQILPDPSKPSKRSFIEDLL |
| | epitope (aa 787-822) | |
| 71. | SARS-CoV-2 S protein | KPSKRSFIEDLLFNKVTLAD |
| | epitope (aa 811-830) | |
| 72. | SARS-CoV-2 S protein | ELDSFKEELDKYFKNHTSPD |
| | epitope (aa 1144-1163) | |
| 73. | SARS-CoV-2 S protein | SFKEELDKYFKN |
| | epitope (aa 1147-1158) | |
| 74. | SARS-CoV-2 S protein | CEFQFCNDPFLGVYYHKNNKSWMESEFRVYSSA |
| | epitope (aa 131-166) | NNC |
| 75. | SARS-CoV-2 S protein | CALDPLSETKC |
| | epitope (aa 291-301) | |
| 76. | SARS-CoV-2 S protein | CSKVGGNYNYLYRLFRKSNLKPFERDISTEIYQAG |
| | epitope (aa 443-495) + | STPCNGVEGFNCYFPLQSYC |
| | flanking cysteine residues | |
| 77. | SARS-CoV-2 S protein | CSKVGGNYNYLYRLFRKSNLKPFERDISTEIYQAG |
| | epitope (aa 443-495_ | STPCNGVKGFNCYFPLQSYC |
| | E484K) + flanking | |
| | cysteine residues | |
| 78. | SARS-CoV-2 S protein | CNGVKGFNC |
| | epitope | |
| | (aa 480-488_E484K) | |
| 79. | SARS-CoV-2 S protein | CVNFNFNGLTGTGVLTESNKKFLPFQQFGRDIAD |
| | epitope (aa 538-590) | TTDAVRDPQTLEILDITPC |
| 80. | SARS-CoV-2 S protein | CTEVPVAIHADQLTPTWRVYSTGSNVFQTRAGC |
| | epitope (aa 617-649) | |
| 81. | Aerosol delivery peptide | tgtgcgaaaagcatgggcgatatcgtgtgt |
| | nucleic acid | |
| 86. | RGD4C-targeting peptide | CDCRGDCFC |
| 111. | SARS-CoV-2 S protein | PINLVRDLPQGFSALEPL |
| | epitope (aa 209-226) | |
| 120. | SARS-CoV-2 S protein | YGFQPTNGVGYQPYRVVVLSFELLHAP |
| | epitope (aa 495-521) | |
| 124. | SARS-CoV-2 S protein | GIAVEQDKNTQEVFAQVK |
| | epitope (aa 769-786) | |
| 126. | SARS-CoV-2 S protein | MAYRFNGIGVTQNVLYENQKLIANQ |
| | epitope (aa 902-926) | |
| 135. | SARS-CoV-2 S protein | NRALTGIAVEQDKNTQEVFAQVKQIYKTPPIKDF |
| | epitope (aa 764-829) | GGFNFSQILPDPSKPSKRSFIEDLLFNKVTLA |
| 136. | SARS-CoV-2 S protein | DEVRQIAPGQTGKIADYNYKLPDDFTGCVIAWNS |
| | epitope (aa 405-469) | NNLDSKVGGNYNYLYRLFRKSNLKPFERDIS |

Mouse Hepatitis Virus (MHV), type 1 Spike (S) Protein Nucleotide Sequence (SEQ ID NO: 82)

Sequence covers 70-4161 nucleotides with 5′ and 3′ flanking sequences from AAVP genomic DNA (bold) and 5′ EcoRI and 3′ SalI restriction sites (underline). Bold, underlined, italic nucleotides show a change of “aat to aac” to delete an EcoRT sequence in the S gene at 148-150. The translated sequence of the MI-IV type 1 S protein shows the corresponding amino acid (N) at position 27 is unchanged (underline).

| | |
|---|---|
| 1 | gaattctgaa tgaaatatac aagttatatc ttggcttttc agctctgcat |
| 51 | cgttttgggt tctcttggca tgctgtttgt cgtgtttatt ctcctaatac |
| 101 | cctcttgttt agggtatatt ggtgacttta gatgtatcca gctcgtg [symbol] |
| 151 | tcaaacggca acaacgcttc tgcgccaagc attagcattg aaactgtcga |
| 201 | tgtttccaaa ggccttggta cttattatgt tttagatcga gtttatttaa |
| 251 | atgccacatt attgcttact ggttattatc ctgtggacgg ttccaattat |
| 301 | aggaatctcg cgcttacagg cactaatacc ctaagcctta attggtataa |
| 351 | accacccttt ttatcagagt ttaatgatgg catatttgct aaggtaaaga |
| 401 | accttaaagc atctctgccc gctggctcat cggcttattt ccctactatt |
| 451 | ataataggca gtggttttgg taacaccgcc tatactatag taatggaacc |
| 501 | atataatggt ataattatgg catctatttg ccagtacacc atttgtcaat |
| 551 | taccgtatac tgattgtaaa cctaatacag gcggtaatag tattataggt |
| 601 | ttttggcaca cagatataaa atcccctgtg tgcattttaa agcgtaattt |
| 651 | cacgtttaat gttaatgccg attggctcta ttttcatttt taccaacagg |
| 701 | gtggtacttt ttatgcgtat tatgcagatg tagcttctgc tactacgttt |
| 751 | ttatttagta tttatattgg cgatgtttta acgcaattct ttgtgttgcc |
| 801 | ttttaattgt gaacctgata aggctggtgt tatatcaccg cagtattggg |
| 851 | tcacaccttt agttgagcgc caatatttgt ttaattttaa ccaaaagggt |
| 901 | attattacta gtgctgttga ttgtgctagt agttataccg ctgaaattaa |
| 951 | atgcaagact caaagtatga atcctagtac gggagtctat gatctcactg |
| 1001 | gttacactgt tcaacctgtt ggtttagtgt accgaagagt tagaaatttg |
| 1051 | cctgattgta aaatagagga ttggctcacc gctaaaagcg tgccgtctcc |
| 1101 | tctcaattgg gaacgtaaaa catttcaaaa ttgtaacttt aacctgagca |
| 1151 | gtctattaag atttgtccag gctgagtcac tctcatgtag taatatagat |
| 1201 | gcttccaaag tttatggaat gtgctttggc agcgtatcta tagataaatt |
| 1251 | tgcaataccc aataggagac gcgttgacct ccaaataggc aattctgggt |
| 1301 | ttttgcagtc ttttaattat aaaatagatt caagggcgac ttcttgccag |
| 1351 | ctttattata gtcttgcaca aaataatgtc accgttaata accataaccc |
| 1401 | gtcctcctgg aataggcgat atggatttaa cgatgtggca acatttggta |
| 1451 | gtggtaaaca tgacgttgca tatgctgaag agtgttttac tgttggtaat |
| 1501 | gattattgcc catgtgctaa ccccagcata gtatcgccat gcacgcaaga |
| 1551 | taaacctaag gctgctaatt gtccagtagg tacacgcaat cgagagtgta |
| 1601 | accctctggc gcttggcggt aatttattta agtgcgactg cacatgtaac |
| 1651 | cctagcccac taactaccta cgaccttcgc tgcctccagg ctaggagcat |
| 1701 | gctaggggta ggtgaccatt gtgaaggact tggtgtttta gaagataaat |
| 1751 | gtggtggaag caacgtttgc aattgtactg cagatgcttt tgttggctgg |
| 1801 | tctacggata gttgtctatc caaaggccgc tgccacattt tctcgaattt |
| 1851 | gttattaaat ggcattaata gtggaaccac ttgctccact gatttacagt |
| 1901 | tgcctaatac tgaagtggtt actggcgttt gtgtcaagta tcatctcttc |
| 1951 | ggtattactg gtcaaggtgt ttttaaggag gttaaagccg actactatca |
| 2001 | tagctggcag aatctcttat atgatgttaa tggcaatctg gaaggtttcc |
| 2051 | gcgacatcat taccaataaa acttatacta ttagaagctg ttatagcggg |
| 2101 | cgagtttcgg ctgcatatca tcaagatgca cctgaacctg cgctgctata |
| 2151 | tcgcaattta aaatgtgatt atgtctttaa caacaacatc tcccgtgagg |
| 2201 | agaccccact taactatttt gatagttatt tgggctgtgt tgttaatgct |
| 2251 | gataactcaa ctgaagaagc tgttgctgtg tgtgatctac gtatgggtag |
| 2301 | tggcctttgc gtcaactatt caacgtcaca tcgagctcgc aggtccatca |
| 2351 | gtacgggtta taaattaact acttttgaac catttacagt tagcattgtc |
| 2401 | aatgatagtg ttcagtctgt gggtggatta tatgagatgc aaataccaat |
| 2451 | caattttact ataggacaac accaggagtt cattcaaact agagctccaa |
| 2501 | aggtaactat agattgtgcg gcttttgtct gtggtgatta cacagcatgc |
| 2551 | cggcagcagt tggttgagta tggatcattc tgtgataata ttaatgccat |
| 2601 | tcttggcgag gttaataacc tcatagatac tatgcaactg caggttgcta |
| 2651 | gtgccctgat acaaggtgtc acgctaagtt cccgcttggc tgatggcatt |
| 2701 | ggtggtcaga ttgatgatat taattttagt cctctgttag gctgtctagg |
| 2751 | ttcagattgt ggtgaaggaa ccactgctgc actaaaggga cggtcggtta |
| 2801 | tagaggatat gctgttcgat aaagtcaaac tatcagatgt tggctttgtt |
| 2851 | gaagcatata ataattgcac tggtggtcag gaagtcagag acctactatg |
| 2901 | tgtgcaatct tttaatggca taaaagtgct gcctcctgta ttatccgaga |
| 2951 | gtcagatctc cggatataca gctggtgcta ctgcgtctgc tatgttccca |
| 3001 | ccttggtctg cagccgcggg tgtgccattt tctttaagtg ttcaatatag |
| 3051 | aattaatggt ctgggtgtca ctatgaatgt tcttagtgaa aaccagaaaa |
| 3101 | tgatagctag tgctttcaac aatgcgattg gtgctataca ggagggcttt |
| 3151 | gatgccacta attctgcatt agcaaaaatt caatccgttg tgaatgcaaa |
| 3201 | tgctgaagca cttaataatc tgttgcaaca attgtccaac agatttggtg |
| 3251 | caattagtgc ttctttacag gaaattctat cccgccttga tgctcttgaa |
| 3301 | gcgcaggctc agatagaccg tcttataaat ggcagattaa ctgcacttaa |
| 3351 | tgcatatgtt tctaagcagc tgagtgacat gaccctagtt aaggtaagtg |
| 3401 | ccgctcaagc tatagagaaa gttaatgagt gtgttaaaag ccaatcacct |
| 3451 | aggattaatt tctgtggcaa tggcaatcat atattgtcat tagtccagag |
| 3501 | tgcgccttat ggcttatatt ttatacactt cagctatgtg cctacatcct |
| 3551 | ttacaacggt aaatgtgagt cctggacttt gcatttctgg tgatagagga |
| 3601 | ttagcaccta aagctggata ttttgttcaa gataatggag agtggaagtt |
| 3651 | cactggtagt ggttattact accctgaacc cataaatgat aaaaacagtg |
| 3701 | tcgttatgag tagttgtgca gtaaactaca caaaagcgcc tgaagttttc |
| 3751 | ttgaacactt caataccaaa tctacccgac tttaaggagg agttagataa |
| 3801 | atggtttaag aatcagacgt ccattgcgcc tgatttatct ctcgatttcg |
| 3851 | agaaattaaa tgttactttc ctggacctga ccgatgagat gaacaggatt |
| 3901 | caggagtcaa ttaagaagtt aaatgagagc tacatcaacc tcaaggaagt |
| 3951 | tggcacatat gaaatgtatg tgaaatggcc ttggtacatt tggttgctaa |
| 4001 | ttggattagc tggtgtagct gtttgtgtgt tgttattctt tatatgctgc |
| 4051 | tgcacaggtt gcggctcatg ttgttttaag aaatgtggaa attgttgtga |
| 4101 | tgagtatgga ggacaccagg atagtattgt catccataat atatcctctc |
| 4151 | acgaggattg aggatcctct agagtcgac |

### MHV Type 1 Spike (S) Protein Sequence (SEQ ID NO: 83)

Flanking 5′ and 3′ AAVP sequences are in bold.

| | |
|---|---|
| 1 | EF*MKYTSYI LAFQLCIVLG SLGMLFVVFI LLIPSCLGYI GDFRCIQLV [symbol] |
| 51 | SNGNNASAPS ISIETVDVSK GLGTYYVLDR VYLNATLLLT GYYPVDGSNY |
| 101 | RNLALTGTNT LSLNWYKPPF LSEFNDGIFA KVKNLKASLP AGSSAYFPTI |
| 151 | IIGSGFGNTA YTIVMEPYNG IIMASICQYT ICQLPYTDCK PNTGGNSIIG |
| 201 | FWHTDIKSPV CILKRNFTFN VNADWLYFHF YQQGGTFYAY YADVASATTF |
| 251 | LFSIYIGDVL TQFFVLPFNC EPDKAGVISP QYWVTPLVER QYLFNFNQKG |
| 301 | IITSAVDCAS SYTAEIKCKT QSMNPSTGVY DLTGYTVQPV GLVYRRVRNL |
| 351 | PDCKIEDWLT AKSVPSPLNW ERKTFQNCNF NLSSLLRFVQ AESLSCSNID |
| 401 | ASKVYGMCFG SVSIDKFAIP NRRRVDLQIG NSGFLQSFNY KIDSRATSCQ |
| 451 | LYYSLAQNNV TVNNHNPSSW NRRYGFNDVA TFGSGKHDVA YAEECFTVGN |
| 501 | DYCPCANPSI VSPCTQDKPK AANCPVGTRN RECNPLALGG NLFKCDCTCN |
| 551 | PSPLTTYDLR CLQARSMLGV GDHCEGLGVL EDKCGGSNVC NCTADAFVGW |
| 601 | STDSCLSKGR CHIFSNLLLN GINSGTTCST DLQLPNTEVV TGVCVKYHLF |
| 651 | GITGQGVFKE VKADYYHSWQ NLLYDVNGNL EGFRDIITNK TYTIRSCYSG |
| 701 | RVSAAYHQDA PEPALLYRNL KCDYVFNNNI SREETPLNYF DSYLGCWNA |
| 751 | DNSTEEAVAV CDLRMGSGLC VNYSTSHRAR RSISTGYKLT TFEPFTVSIV |
| 801 | NDSVQSVGGL YEMQIPINFT IGQHQEFIQT RAPKVTIDCA AFVCGDYTAC |
| 851 | RQQLVEYGSF CDNINAILGE VNNLIDTMQL QVASALIQGV TLSSRLADGI |
| 901 | GGQIDDINFS PLLGCLGSDC GEGTTAALKG RSVIEDMLFD KVKLSDVGFV |
| 951 | EAYNNCTGGQ EVRDLLCVQS FNGIKVLPPV LSESQISGYT AGATASAMFP |
| 1001 | PWSAAAGVPF SLSVQYRING LGVTMNVLSE NQKMIASAFN NAIGAIQEGF |
| 1051 | DATNSALAKI QSVVNANAEA LNNLLQQLSN RFGAISASLQ EILSRLDALE |
| 1101 | AQAQIDRLIN GRLTALNAYV SKQLSDMTLV KVSAAQAIEK VNECVKSQSP |
| 1151 | RINFCGNGNH ILSLVQSAPY GLYFIHFSYV PTSFTTVNVS PGLCISGDRG |
| 1201 | LAPKAGYFVQ DNGEWKFTGS GYYYPEPIND KNSVVMSSCA VNYTKAPEVF |
| 1251 | LNTSIPNLPD FKEELDKWFK NQTSIAPDLS LDFEKLNVTF LDLTDEMNRI |
| 1301 | QESIKKLNES YINLKEVGTY EMYVKWPWYI WLLIGLAGVA VCVLLFFICC |
| 1351 | CTGCGSCCFK KCGNCCDEYG GHQDSIVIHN ISSHED*GSS RVD |

### SARS-CoV-2 Spike (S) Glycoprotein Gene Nucleotide Sequence (SEQ ID NO: 84)

Sequence includes 70-3891 nucleotides with 5′ and 3′ flanking sequences from AAVP genomic DNA (bold) and 5′ EcoRI and 3′ SalI restriction sites (underline). Bold, underlined, italic nucleotides show a change of “aat to aac” to delete an EcoRI sequence in the S gene at 1378-1380. The translated sequence of the SARS-CoV-2 Spike protein gene shows the corresponding amino acid (N) at position 460 is unchanged (bold, underline, italic).

| | |
|---|---|
| 1 | gaattctgaa tgaaatatac aagttatatc ttggcttttc agctctgcat |
| 51 | cgttttgggt tctcttggca tgtttgtttt tcttgtttta ttgccactag |
| 101 | tctctagtca gtgtgttaat cttacaacca gaactcaatt accccctgca |
| 151 | tacactaatt ctttcacacg tggtgtttat taccctgaca aagttttcag |
| 201 | atcctcagtt ttacattcaa ctcaggactt gttcttacct ttcttttcca |
| 251 | atgttacttg gttccatgct atacatgtct ctgggaccaa tggtactaag |
| 301 | aggtttgata accctgtcct accatttaat gatggtgttt attttgcttc |
| 351 | cactgagaag tctaacataa taagaggctg gatttttggt actactttag |
| 401 | attcgaagac ccagtcccta cttattgtta ataacgctac taatgttgtt |
| 451 | attaaagtct gtgaatttca attttgtaat gatccatttt tgggtgttta |
| 501 | ttaccacaaa aacaacaaaa gttggatgga aagtgagttc agagtttatt |
| 551 | ctagtgcgaa taattgcact tttgaatatg tctctcagcc ttttcttatg |
| 601 | gaccttgaag gaaaacaggg taatttcaaa aatcttaggg aatttgtgtt |
| 651 | taagaatatt gatggttatt ttaaaatata ttctaagcac acgcctatta |
| 701 | atttagtgcg tgatctccct cagggttttt cggctttaga accattggta |
| 751 | gatttgccaa taggtattaa catcactagg tttcaaactt tacttgcttt |
| 801 | acatagaagt tatttgactc ctggtgattc ttcttcaggt tggacagctg |
| 851 | gtgctgcagc ttattatgtg ggttatcttc aacctaggac ttttctatta |
| 901 | aaatataatg aaaatggaac cattacagat gctgtagact gtgcacttga |
| 951 | ccctctctca gaaacaaagt gtacgttgaa atccttcact gtagaaaaag |
| 1001 | gaatctatca aacttctaac tttagagtcc aaccaacaga atctattgtt |
| 1051 | agatttccta atattacaaa cttgtgccct tttggtgaag tttttaacgc |
| 1101 | caccagattt gcatctgttt atgcttggaa caggaagaga atcagcaact |
| 1151 | gtgttgctga ttattctgtc ctatataatt ccgcatcatt ttccactttt |
| 1201 | aagtgttatg gagtgtctcc tactaaatta aatgatctct gctttactaa |
| 1251 | tgtctatgca gattcatttg taattagagg tgatgaagtc agacaaatcg |
| 1301 | ctccagggca aactggaaag attgctgatt ataattataa attaccagat |
| 1351 | gattttacag gctgcgttat agcttgg [symbol] tctaacaatc ttgattctaa |
| 1401 | ggttggtggt aattataatt acctgtatag attgtttagg aagtctaatc |
| 1451 | tcaaaccttt tgagagagat atttcaactg aaatctatca ggccggtagc |
| 1501 | acaccttgta atggtgttga aggttttaat tgttactttc ctttacaatc |
| 1551 | atatggtttc caacccacta atggtgttgg ttaccaacca tacagagtag |
| 1601 | tagtactttc ttttgaactt ctacatgcac cagcaactgt ttgtggacct |
| 1651 | aaaaagtcta ctaatttggt taaaaacaaa tgtgtcaatt tcaacttcaa |
| 1701 | tggtttaaca ggcacaggtg ttcttactga gtctaacaaa aagtttctgc |
| 1751 | ctttccaaca atttggcaga gacattgctg acactactga tgctgtccgt |
| 1801 | gatccacaga cacttgagat tcttgacatt acaccatgtt cttttggtgg |
| 1851 | tgtcagtgtt ataacaccag gaacaaatac ttctaaccag gttgctgttc |
| 1901 | tttatcagga tgttaactgc acagaagtcc ctgttgctat tcatgcagat |
| 1951 | caacttactc ctacttggcg tgtttattct acaggttcta atgtttttca |
| 2001 | aacacgtgca ggctgtttaa taggggctga acatgtcaac aactcatatg |
| 2051 | agtgtgacat acccattggt gcaggtatat gcgctagtta tcagactcag |
| 2101 | actaattctc ctcggcgggc acgtagtgta gctagtcaat ccatcattgc |
| 2151 | ctacactatg tcacttggtg cagaaaattc agttgcttac tctaataact |
| 2201 | ctattgccat acccacaaat tttactatta gtgttaccac agaaattcta |
| 2251 | ccagtgtcta tgaccaagac atcagtagat tgtacaatgt acatttgtgg |
| 2301 | tgattcaact gaatgcagca atcttttgtt gcaatatggc agtttttgta |
| 2351 | cacaattaaa ccgtgcttta actggaatag ctgttgaaca agacaaaaac |
| 2401 | acccaagaag tttttgcaca agtcaaacaa atttacaaaa caccaccaat |
| 2451 | taaagatttt ggtggtttta atttttcaca aatattacca gatccatcaa |
| 2501 | aaccaagcaa gaggtcattt attgaagatc tacttttcaa caaagtgaca |
| 2551 | cttgcagatg ctggcttcat caaacaatat ggtgattgcc ttggtgatat |
| 2601 | tgctgctaga gacctcattt gtgcacaaaa gtttaacggc cttactgttt |
| 2651 | tgccaccttt gctcacagat gaaatgattg ctcaatacac ttctgcactg |
| 2701 | ttagcgggta caatcacttc tggttggacc tttggtgcag gtgctgcatt |
| 2751 | acaaatacca tttgctatgc aaatggctta taggtttaat ggtattggag |
| 2801 | ttacacagaa tgttctctat gagaaccaaa aattgattgc caaccaattt |
| 2851 | aatagtgcta ttggcaaaat tcaagactca ctttcttcca cagcaagtgc |
| 2901 | acttggaaaa cttcaagatg tggtcaacca aaatgcacaa gctttaaaca |
| 2951 | cgcttgttaa acaacttagc tccaattttg gtgcaatttc aagtgtttta |
| 3001 | aatgatatcc tttcacgtct tgacaaagtt gaggctgaag tgcaaattga |
| 3051 | taggttgatc acaggcagac ttcaaagttt gcagacatat gtgactcaac |
| 3101 | aattaattag agctgcagaa atcagagctt ctgctaatct tgctgctact |
| 3151 | aaaatgtcag agtgtgtact tggacaatca aaaagagttg atttttgtgg |
| 3201 | aaagggctat catcttatgt ccttccctca gtcagcacct catggtgtag |
| 3251 | tcttcttgca tgtgacttat gtccctgcac aagaaaagaa cttcacaact |
| 3301 | gctcctgcca tttgtcatga tggaaaagca cactttcctc gtgaaggtgt |
| 3351 | ctttgtttca aatggcacac actggtttgt aacacaaagg aatttttatg |
| 3401 | aaccacaaat cattactaca gacaacacat ttgtgtctgg taactgtgat |
| 3451 | gttgtaatag gaattgtcaa caacacagtt tatgatcctt tgcaacctga |
| 3501 | attagactca ttcaaggagg agttagataa atattttaag aatcatacat |
| 3551 | caccagatgt tgatttaggt gacatctctg gcattaatgc ttcagttgta |
| 3601 | aacattcaaa aagaaattga ccgcctcaat gaggttgcca agaatttaaa |
| 3651 | tgaatctctc atcgatctcc aagaacttgg aaagtatgag cagtatataa |
| 3701 | aatggccatg gtacatttgg ctaggtttta tagctggctt gattgccata |
| 3751 | gtaatggtga caattatgct ttgctgtatg accagttgct gtagttgtct |
| 3801 | caagggctgt tgttcttgtg gatcctgctg caaatttgat gaagacgact |
| 3851 | ctgagccagt gctcaaagga gtcaaattac attacacata aggatcctct |
| 3901 | agagtcgac |

### SARS-CoV-2 Spike (S) Glycoprotein Sequence (SEQ ID NO: 85)

Flanking 5′ and 3′ AAVP sequences are in bold.

| | |
|---|---|
| 1 | EF*MKYTSYI LAFQLCIVLG SLGMFVFLVL LPLVSSQCVN LTTRTQLPPA |
| 51 | YTNSFTRGVY YPDKVFRSSV LHSTQDLFLP FFSNVTWFHA IHVSGTNGTK |
| 101 | RFDNPVLPFN DGVYFASTER SNIIRGWIFG TTLDSKTQSL LIVNNATNVV |
| 151 | IKVCEFQFCN DPFLGVYYHK NNKSWMESEF RVYSSANNCT FEYVSQPFLM |
| 201 | DLEGKQGNFK NLREFVFKNI DGYFKIYSKH TPINLVRDLP QGFSALEPLV |
| 251 | DLPIGINITR FQTLLALHRS YLTPGDSSSG WTAGAAAYYV GYLQPRTFLL |
| 301 | KYNENGTITD AVDCALDPLS ETKCTLKSFT VEKGIYQTSN FRVQPTESIV |
| 351 | RFPNITNLCP FGEVFNATRF ASVYAWNRKR ISNCVADYSV LYNSASFSTF |
| 401 | KCYGVSPTKL NDLCFTNVYA DSFVIRGDEV RQIAPGQTGR IADYNYKLPD |
| 451 | DFTGCVIAW [symbol] SNNLDSKVGG NYNYLYRLFR KSNLKPFERD ISTEIYQAGS |
| 501 | TPCNGVEGFN CYFPLQSYGF QPTNGVGYQP YRVVVLSFEL LHAPATVCGP |
| 551 | KKSTNLVKNK CVNFNFNGLT GTGVLTESNK KFLPFQQFGR DIADTTDAVR |
| 601 | DPQTLEILDI TPCSFGGVSV ITPGTNTSNQ VAVLYQDVNC TEVPVAIHAD |
| 651 | QLTPTWRVYS TGSNVFQTRA GCLIGAEHVN NSYECDIPIG AGICASYQTQ |
| 701 | TNSPRRARSV ASQSIIAYTM SLGAENSVAY SNNSIAIPTN FTISVTTEIL |
| 751 | PVSMTKTSVD CTMYICGDST ECSNLLLQYG SFCTQLNRAL TGIAVEQDKN |
| 801 | TQEVFAQVKQ IYKTPPIKDF GGFNFSQILP DPSRPSRRSF IEDLLFNKVT |
| 851 | LADAGFIKQY GDCLGDIAAR DLICAQKFNG LTVLPPLLTD EMIAQYTSAL |
| 901 | LAGTITSGWT FGAGAALQIP FAMQMAYRFN GIGVTQNVLY ENQRLIANQF |
| 951 | NSAIGKIQDS LSSTASALGK LQDVVNQNAQ ALNTLVKQLS SNFGAISSVL |
| 1001 | NDILSRLDKV EAEVQIDRLI TGRLQSLQTY VTQQLIRAAE IRASANLAAT |
| 1051 | KMSECVLGQS KRVDFCGKGY HLMSFPQSAP HGVVFLHVTY VPAQEKNFTT |
| 1101 | APAICHDGKA HFPREGVFVS NGTHWFVTQR NFYEPQIITT DNTFVSGNCD |
| 1151 | VVIGIVNNTV YDPLQPELDS FKEELDKYFK NHTSPDVDLG DISGINASVV |
| 1201 | NIQKEIDRLN EVAKNLNESL IDLQELGKYE QYIKWPWYIW LGFIAGLIAI |
| 1251 | VMVTIMLCCM TSCCSCLRGC CSCGSCCRFD EDDSEPVLKG VRLHYT*GSS |
| 1301 | RVD |

### Example 1: Lymph Node (LN)-Homing Phage Elicits a Stronger Humoral Immune Response than Untargeted Control Phage

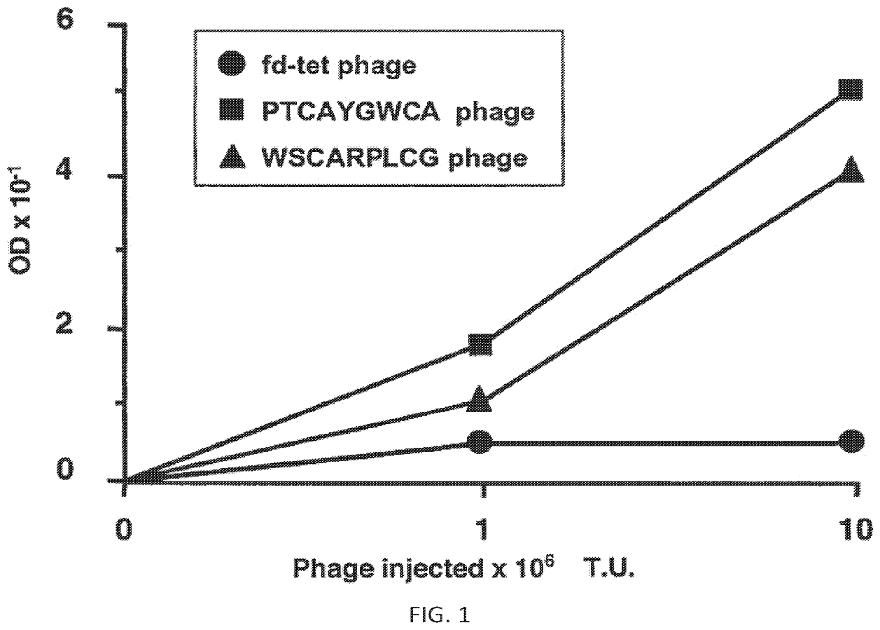

Female two-month-old BALB/c mice received i.v. injections of phage displaying PTCAYGWCA, WSCARPLCG, or no peptide (fd-tet phage, negative control) as indicated (FIG. 1). Anti-phage antibody serum titters were determined by ELISA. Shown are the humoral immune responses three days after the second vaccination with serum dilutions of 1:500. Data represent the absorbance (A450 nm) of the p-nitrophenyl phosphate substrate (Trepel et al., 2001).

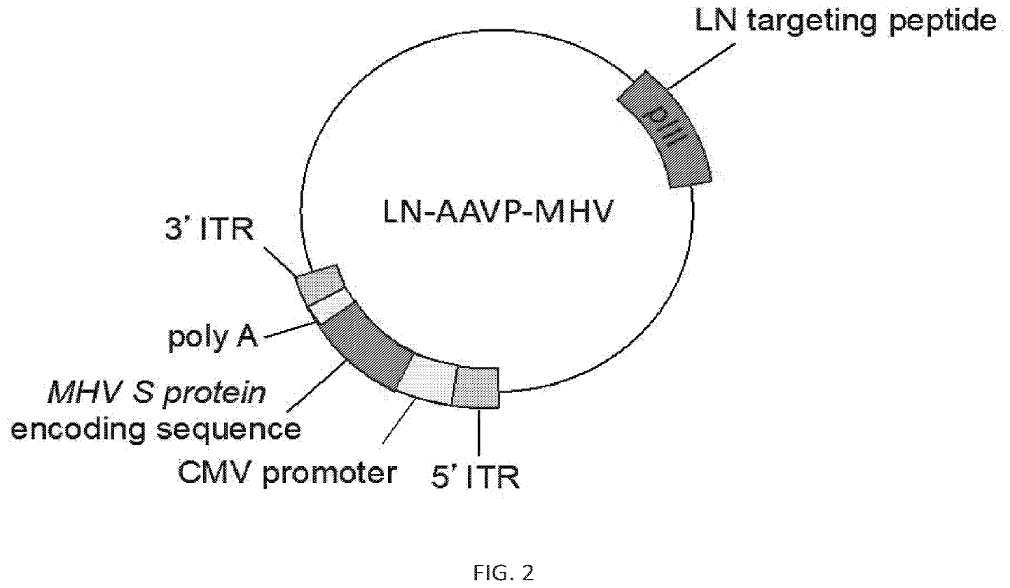

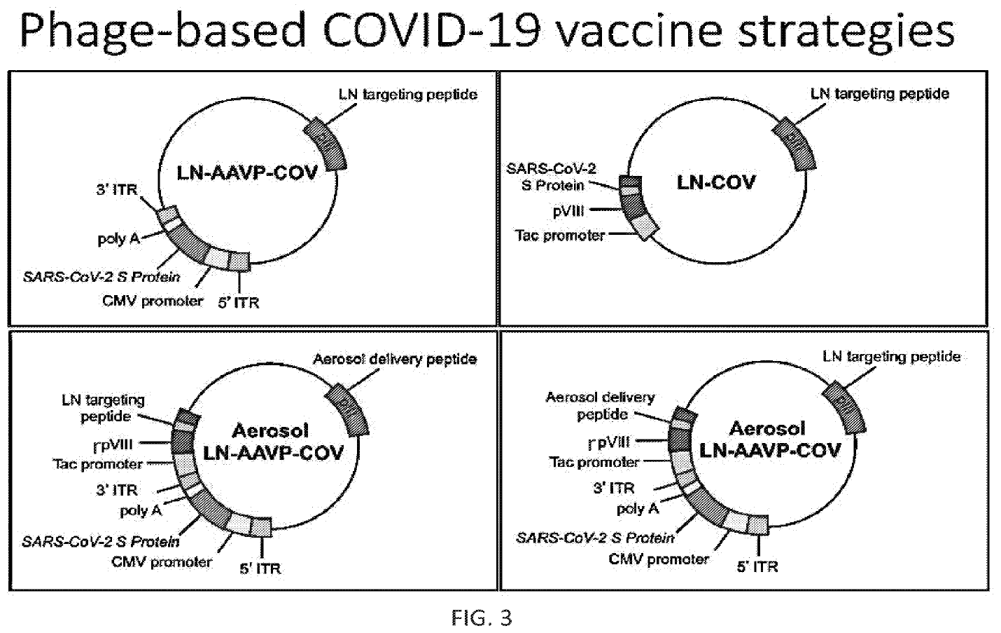

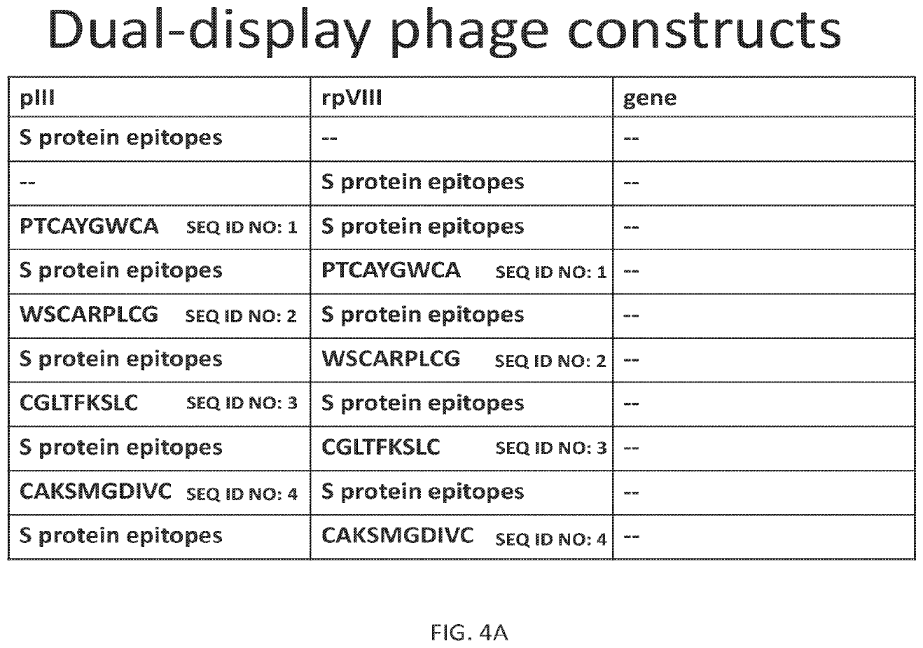

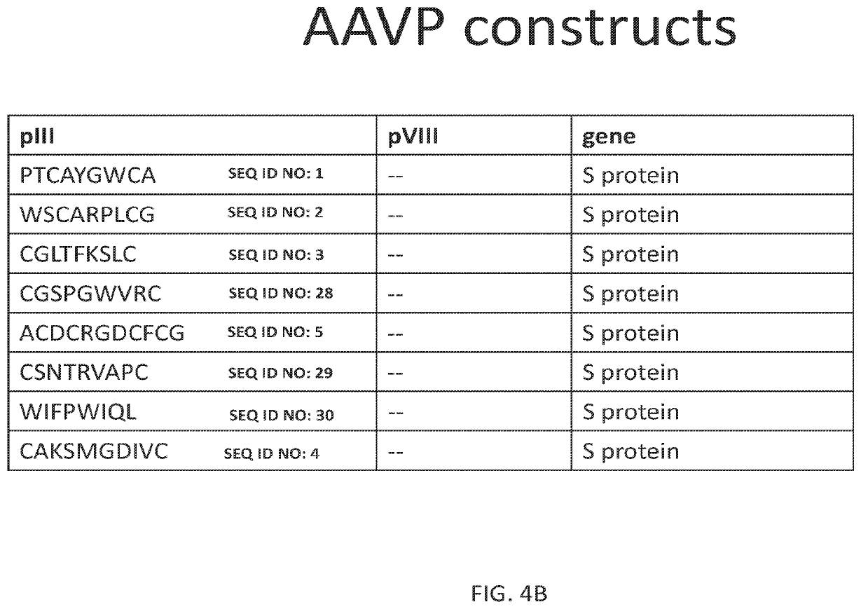

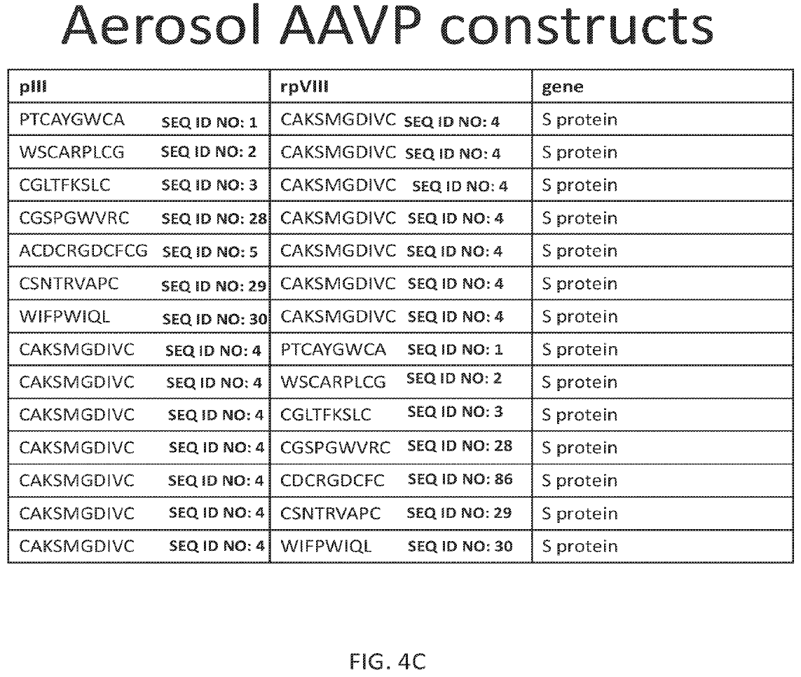

FIG. 2 illustrates a map of a LN-targeting AAVP designed to generate an immune response against the MHV type 1 S protein. Targeted AAVP vectors delivering either gene encoding for the MHV type 1 S protein or control antigen are generated using routine molecular biology strategies. To enhance the immune response, LN-targeting peptides are expressed in the pIII minor coat protein. FIGS. 3 and 4 illustrate additional constructs designed to generate an immune response against the SARS-CoV-2 S protein.

### Example 2: Detection of Antibodies Against S Protein Epitopes in Human COVID-19 Patients

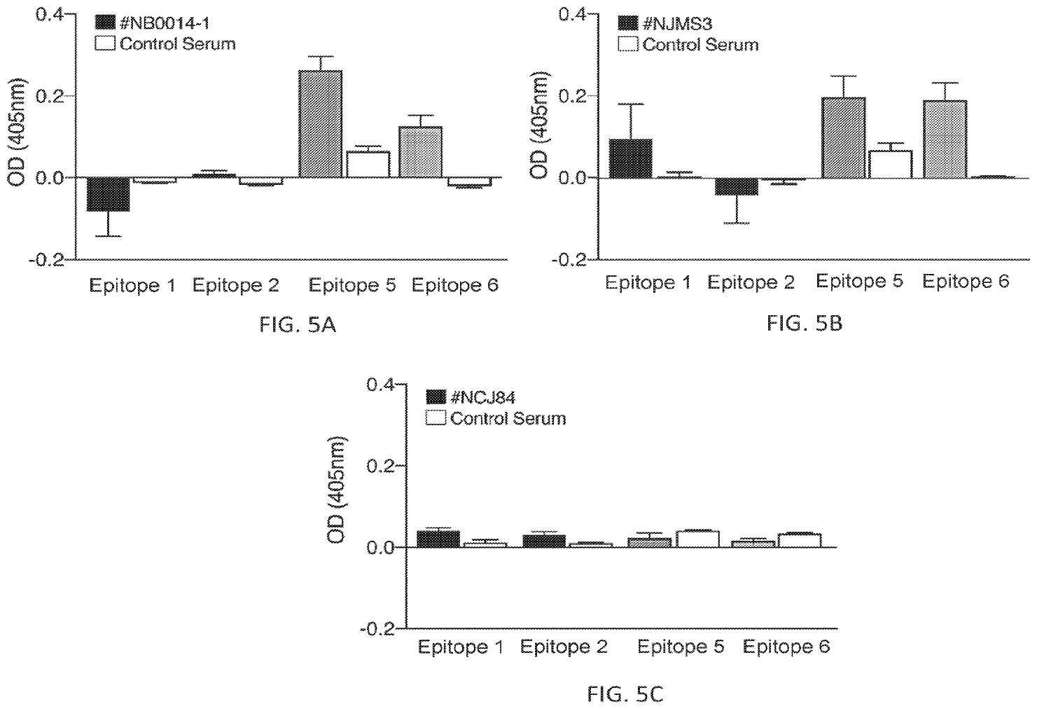

The clinical relevance of two of the epitopes of the disclosure was then assessed in human COVID-19 patients. Antibodies against epitope 5 (SEQ ID NO: 26) and epitope 6 (SEQ ID NO: 27) were detected in the serum of two out of three patients recovered from COVID-19 (FIGS. 5A-5B). Normal serum was used as control. Background values of non-specific binding to wild type bacteriophage were subtracted from all other experimental conditions. (FIG. 5C).

### Example 3: Development of Novel Phage- and AAVP-Based Vaccine Platforms Against SARS-CoV-2

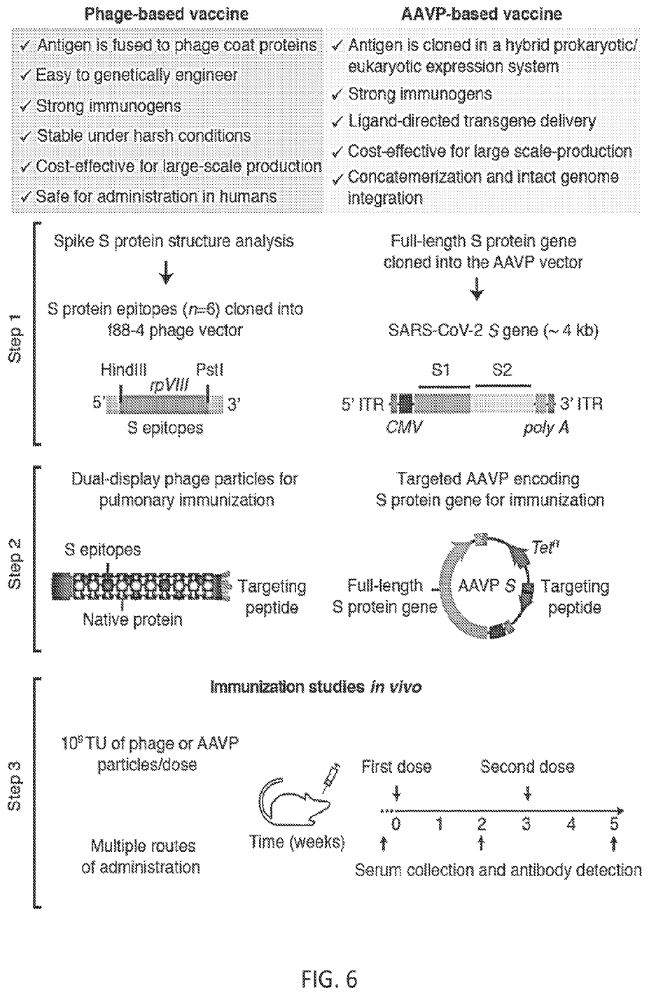

In the present disclosure, two different phage-based vaccine strategies were pursued: 1) ligand-directed phage vaccine candidates displaying different S protein epitopes, and 2) an AAVP-based vaccine candidate against the entire SARS-CoV-2 S protein (FIG. 6). For both strategies, a ligand peptide was incorporated along with the viral antigens in the phage or AAVP to target specific cell surface receptors and facilitate the development of the immune response.

For the first strategy, phage were genetically engineered to display immunologically-relevant S protein epitopes (see below) on the highly exposed rpVIII major coat protein of the phage capsid using the f88-4 vector (FIG. 6, Step 1). To enable tissue-specific targeting of these phage particles (FIG. 6, Step 2), the coding sequence of the novel peptide ligand CAKSMGDIVC (SEQ ID NO: 4) was also subcloned into the pIII minor coat protein gene of the fUSE55 vector, to produce a dual-display phage. The CAKSMGDIVC (SEQ ID NO: 4) ligand binds to α3β1 integrins and mediates the transport of phage particles across the lung epithelium to the systemic circulation where they elicit strong and sustained pulmonary and systemic humoral responses against antigens displayed on the phage capsid (Staquicini et al., 2020). As a control, the untargeted parental insertless phage particles that display the native pVIII and pIII proteins were used.

For the second strategy, the expression cassette containing the full-length S protein transgene and the human CMV promoter was inserted in cis within the 5′ and 3′ ITRs in the AAVP genome for gene delivery and transduction in host cells (FIG. 6, Step 2). This approach allows the rapid “swapping” of targeting motifs and gene coding sequences, providing valuable flexibility to design a variety of vaccines and overcome potential limitations in protein conformation of structure-designed epitopes. As a control, the targeted AAVP empty vector (termed AAVP S-null) was used. Targeting was afforded by the display of a different integrin-binding peptide, ACDCRGDCFCG (RGD4C) (SEQ ID NO: 5), which has been well described for its high affinity to αv integrins and are highly expressed in leukocytes trafficking to draining lymph nodes and areas of inflammation. The RGD motif (arginine-glycine-aspartate) facilitates particle uptake by dendritic cells and enhances the immunogenicity of peptide antigens, DNA vaccines, and adenovirus vectors.

The dual-display phage particles, the RGD4C AAVP S particles, and their corresponding controls were tested in vivo in mice to assess different routes of administration, and to evaluate the induced antigen-specific humoral response by ELISA (FIG. 6, Step 3). The overall vaccination schedule included at least two administered doses of 10⁹ transducing units (TU) of phage or AAVP particles within an interval of 1-2 weeks.

### Example 4: Identification and Selection of Epitopes for Dual-Display Phage-Based Vaccine

To identify relevant epitopes for the first strategy, in silico analysis of the experimentally-determined viral S protein structures of the Wuhan-Hu-1 strain (GenBank Accession number: NC_045512.2) was used. Solvent-exposed amino acid stretches with flanking cysteine residues and cyclic conformation were prioritized because these amino acid sequences are more likely to recapitulate endogenous epitope conformations and therefore increase the likelihood of antigen recognition and processing by the host immune system. Other epitopes were also considered following structural predictions, even in the absence of flanking cysteine residues. Also, given that phage particles are produced in prokaryotic host cells, epitopes with no sites of predicted post-translational modifications were prioritized.

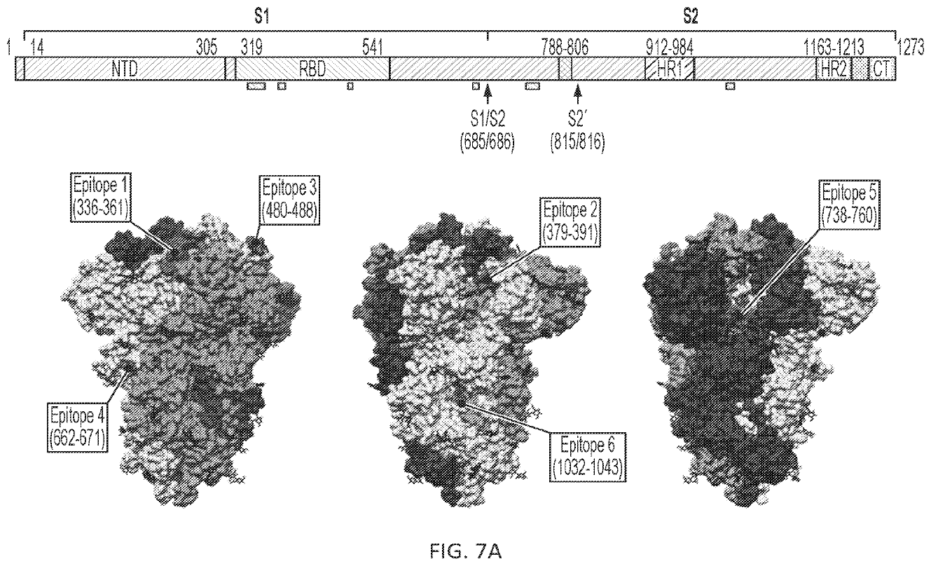

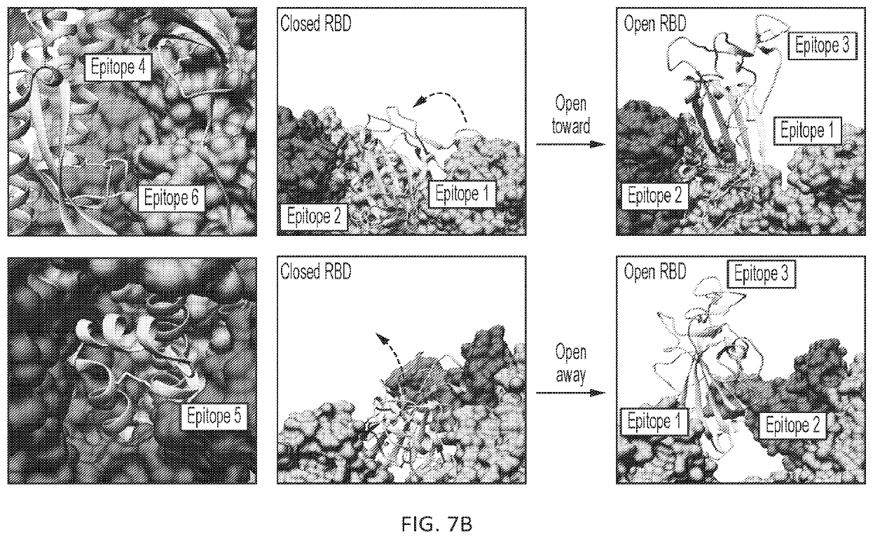

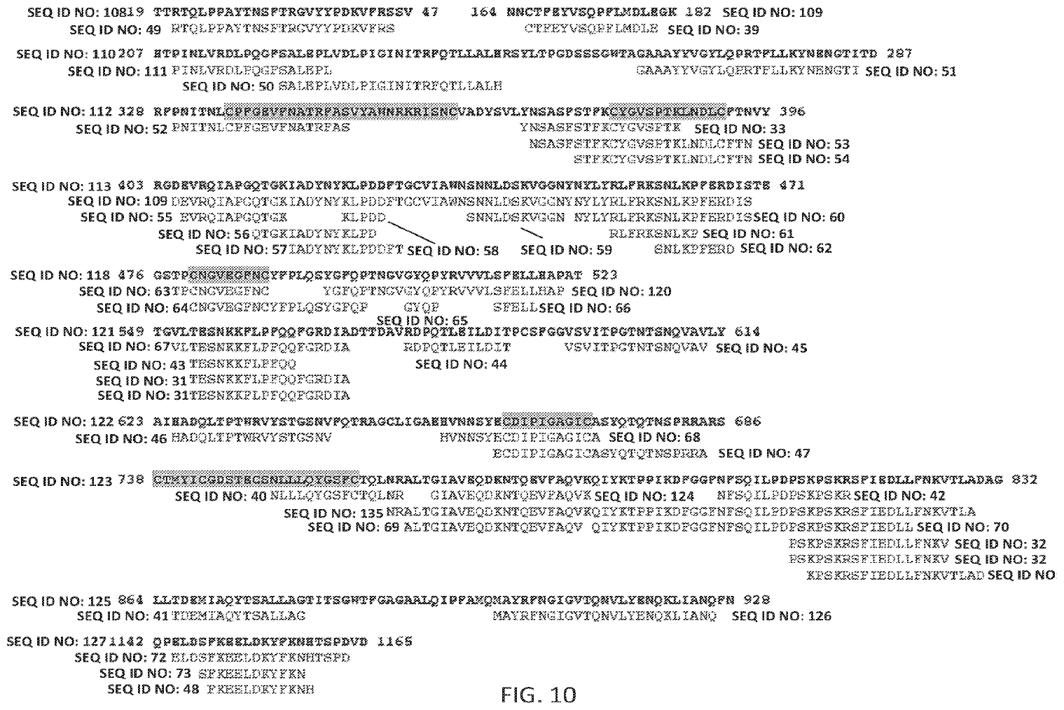

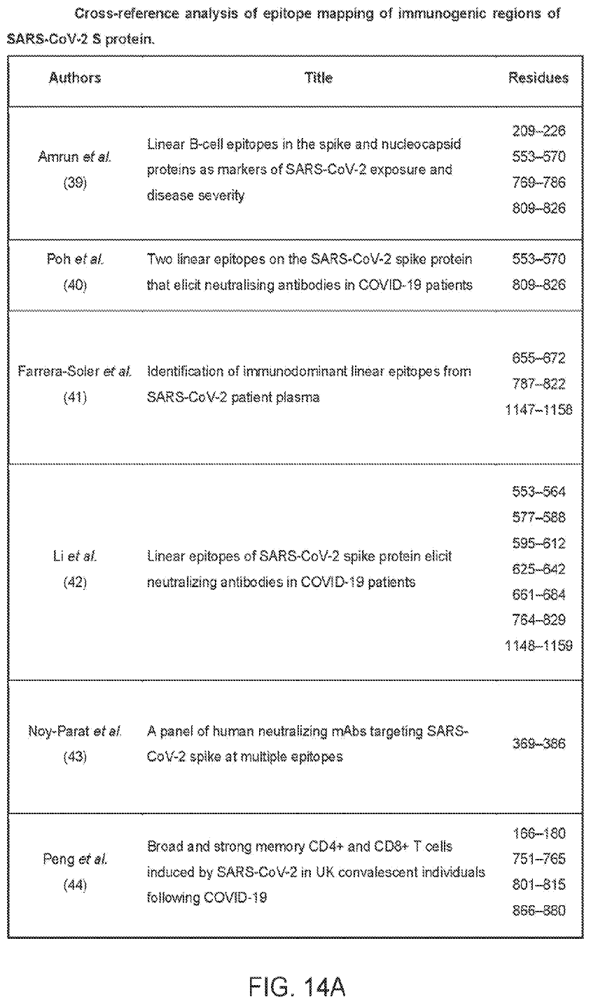

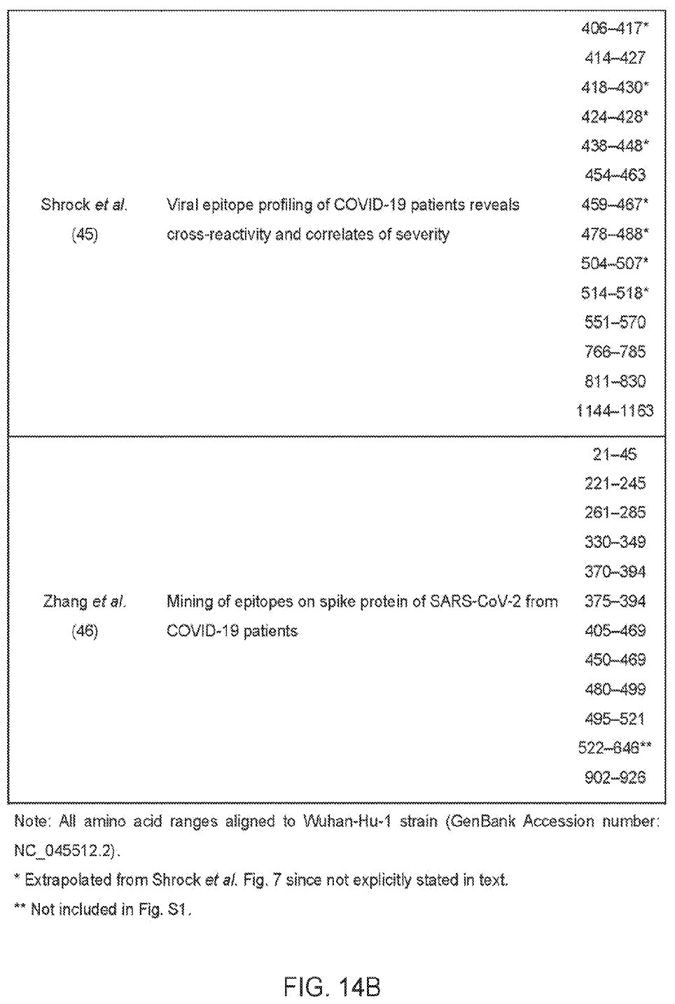

Six S protein epitopes were selected, which are accessible in both closed- and open-state S protein. At least five of these epitopes have since been shown to be fully or partially immunogenic (FIGS. 10 and 14). The six epitopes range from 9 to 26 amino acids (aa) in length. Four are located in the S1 subunit and two in the S2 subunit (FIG. 7A). Three of the S1 epitopes are located in the receptor-binding domain (RBD): epitope 1 (SEQ ID NO: 22), epitope 2 (SEQ ID NO: 23), and epitope 3 (SEQ ID NO: 24). The last epitope of the S1 subunit, epitope 4 (SEQ ID NO: 25), is located adjacent to the site of cleavage between the S1 and S2 subunits. The epitopes of the S2 subunit, epitope 5 (SEQ ID NO: 26) and epitope 6 (SEQ ID NO: 27), are located near fusion peptide (FP) (aa 788-806) and heptapeptide repeat sequence 1 (HR1) (aa 912-984), respectively. Most of the selected epitopes are cyclic in conformation due to the flanking cysteine residues, except epitope 2 (SEQ ID NO: 23), which maintains a loop-like conformation despite the absence of disulfide bridges (FIG. 7B).

Many studies have demonstrated that the S protein is highly glycosylated and some of the glycosylation sites have been reported to alter the infectivity of variants and facilitate evasion of the host immune response (Walls et al. (2020) Cell. 181, 281-292); Li et al. (2020) Cell. 182, 1284-1294). Considering that epitopes were selected based on conformation, epitope 1 (SEQ ID NO: 22) was also identified to contain a glycosylation site on residue N343. Notably, this site seems to be important in viral infectivity in which the glycosylation deletion N343Q drastically reduces the D614G variant infectivity (Li et al. (2020) Cell. 182, 1284-1294). In the present system, however, and without wishing to be bound by theory, the lack of the N-glycosylation is expected to not produce a significant structural divergence in the epitope conformation when displayed on the phage capsid, as similarly observed with other SARS-CoV-2 strains (Kumar et al. (2020) Virusdisease. 31, 13-21). Because the glycosylation site of epitope 1 (SEQ ID NO: 22) is located in its N-terminus and unlikely to interrupt antibody recognition of the remaining structure, it was then sought to investigate the efficacy of this epitope as well.

### Example 5: Characterization of Structurally-Defined S Epitopes for Immunogenicity in Mice

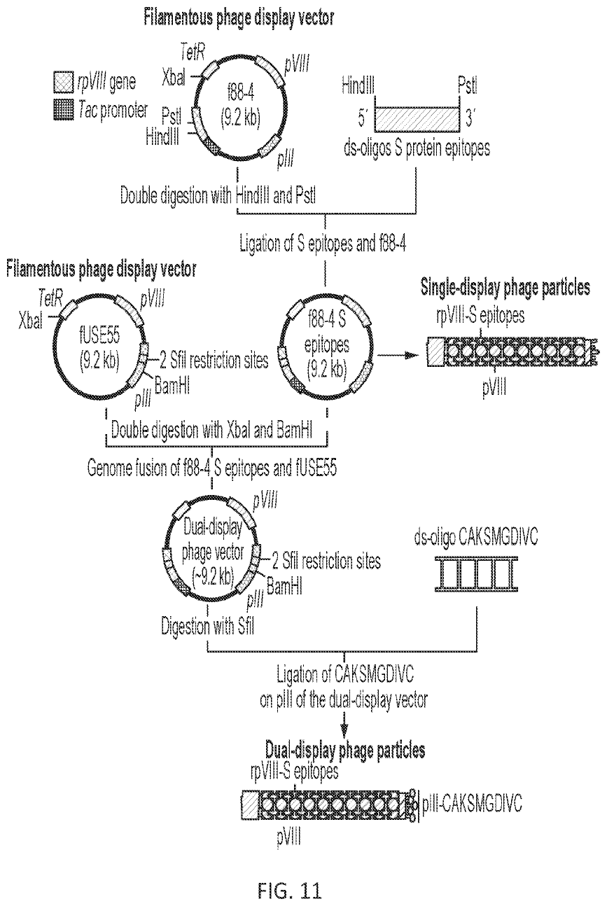

To evaluate the immunological potential of each of the S epitopes and select the most promising candidate(s) for the development of a phage-based vaccine, their ability to induce an immune response was tested in mice. To increase the epitope display on the phage capsid, the parental f88-4 phage genome was used that contains two capsid genes encoding the wild-type pVIII protein and a recombinant pVIII (rpVIII). Six f88-4 phage were produced, each exhibiting one of the six S protein epitopes fused into the rpVIII protein on ˜300 copies per phage particle (termed single-display phage) (FIG. 11).

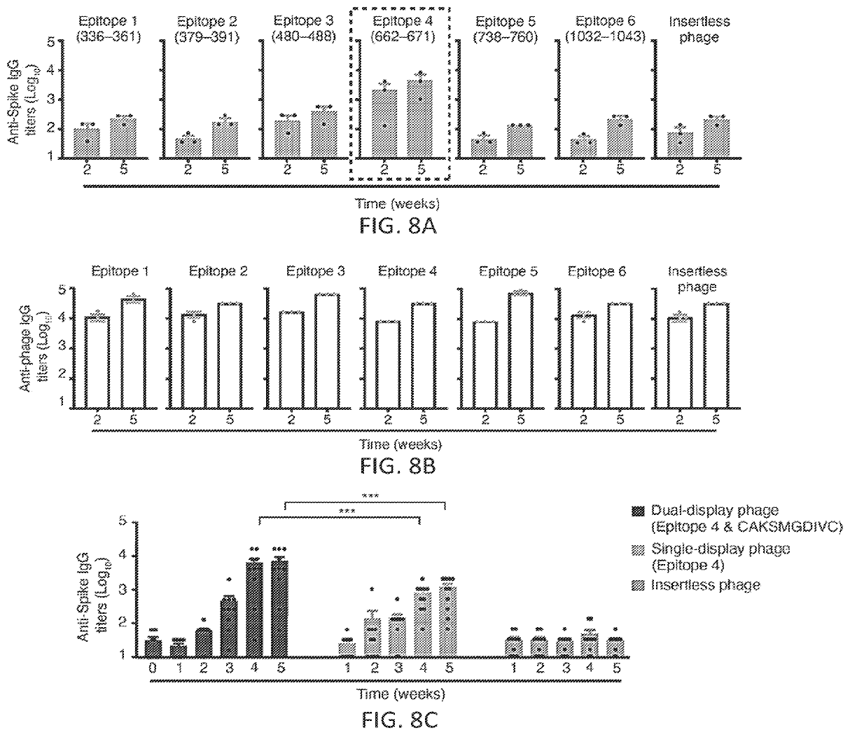

The immunogenicity of the epitopes expressed on the rpVIII phage capsid was assessed in mice serum (Swiss Webster or BALB/c) obtained after the first dose (prime) and the second dose (boost) and compared against the control insertless phage. Antigen-specific IgG titers were quantified by ELISA using an immobilized recombinant full-length SARS-CoV-2 S protein (aa 16-1213) for capture. Epitope 4 (SEQ ID NO: 25) from the S1 subunit induced high levels of S protein-specific IgG antibodies, and booster immunizations further increased antibody levels (FIG. 8A). The other five phage constructs did not induce significant production of S protein-specific IgG antibodies, as similarly observed for mice immunized with the control insertless phage. These results indicate that epitope 4 (SEQ ID NO: 25) is the most immunogenic among the selected epitopes, suggesting that epitope display on its native conformation is necessary for the development of specific immune response as predicted by the in silico analysis.

Given the well-documented inherent immunogenicity of native filamentous phage, the levels of phage-specific IgG antibodies in the serum of these mice were also investigated. Notably, all single-display phage constructs generated high titers of phage-specific IgG antibodies, which were markedly increased after the second dose in the presence or absence of the S protein epitopes displayed on the rpVIII capsid (FIG. 8B). Of note, the generation of phage-specific IgG antibodies does not seem to compromise the S protein-specific humoral response since a clear distinction was observed between epitope 4 (SEQ ID NO: 25) and the other phage particles, including the control insertless phage. Therefore, epitope 4 (SEQ ID NO: 25) was selected as the lead candidate to test in the dual-display phage-based vaccine development by adding a targeting moiety.

### Example 6: Dual-Display Phage Construct for Pulmonary Vaccination

To generate a dual-display phage vaccine, a simple two-step cloning strategy was optimized that allows the rapid exchange of epitopes and/or targeting ligand peptides on the phage genome to generate highly efficient vaccines that can mitigate any potential mutation in epitopes and/or direct the phage to target cells or tissue to improve immune response. Given that pulmonary vaccination has been shown to be the most efficient route to generate mucosal and systemic immunity against airborne pathogens and to significantly increase immunological protection in the upper and lower respiratory tracts of non-human primates challenged with SARS-CoV-2, the peptide CAKSMGDIVC (SEQ ID NO: 4) was elected to use as a lung epithelium-targeted motif. This peptide enables the selective targeting and transport of aerosolized phage particles across the lung barriers while elicits a local and systemic immune response against proteins of the phage capsid without any side effects. Thus, dual-display phage particles that simultaneously express epitope 4 (SEQ ID NO: 25) on the rpVIII (˜300 copies) and the peptide CAKSMGDIVC (SEQ ID NO: 4) on pIII (3-5 copies) were generated.

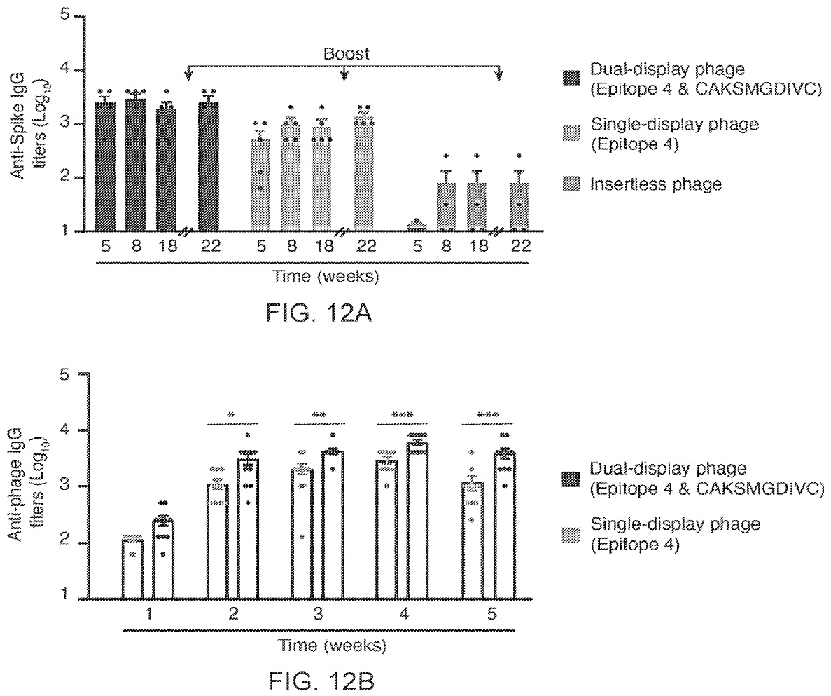

To assess the immunogenicity of the epitope 4 (SEQ ID NO: 25)/CAKSMGDIVC (SEQ ID NO: 4) dual-display phage particles, groups of five-week-old BALB/c female mice were immunized intratracheally. Cohorts of mice (n=10 per group) received two doses of 10⁹ TU of the dual-display phage, the single-display phage, or the control insertless phage in 3-week intervals. Epitope-specific IgG antibodies were evaluated in serum samples collected weekly against the recombinant S protein by ELISA. Titers of S protein-specific IgG antibodies were higher in mice immunized with the epitope 4 (SEQ ID NO: 25)/CAKSMGDIVC (SEQ ID NO: 4) dual-display phage particles than the controls, especially after three weeks post-immunization, and with a substantial increase after the second dose (weeks four and five) (FIG. 8C). These levels remained elevated for over 18 weeks post-immunization with no detectable increases after another boost (FIG. 12A). Notably, single-display phage particles (epitope 4 only; SEQ ID NO: 25) also induced systemic S protein-specific IgG antibodies, but levels were lower than the dual-display phage particles. These results suggest that the addition of the CAKSMGDIVC (SEQ ID NO: 4) peptide permits the transport of the dual-display phage to the systemic circulation, increasing the immunogenicity. As expected, dual-display phage particles also induced a strong and sustained anti-phage humoral response (FIG. 12B), which firmly establishes that epitope 4/CAKSMGDIVC dual-display phage particles induce higher levels of antibody response relative to epitope 4 single-display phage particles.

Together, the data demonstrate that epitope 4 (SEQ ID NO: 25) induces a robust S protein-specific humoral response when displayed on the rpVIII capsid of phage, as a single display entity (single-display phage particles) and, when combined with the lung transport peptide CAKSMGDIVC (SEQ ID NO: 4), this immunogenicity is enhanced. This provides promising evidence that epitope 4 (SEQ ID NO: 25)/CAKSMGDIVC (SEQ ID NO: 4) dual-display phage particles are a suitable candidate for pulmonary vaccination against SARS-CoV-2.

### Example 7: A Novel AAVP-Based Vaccine for Efficient Gene Delivery and Humoral Response Against the Viral S Protein

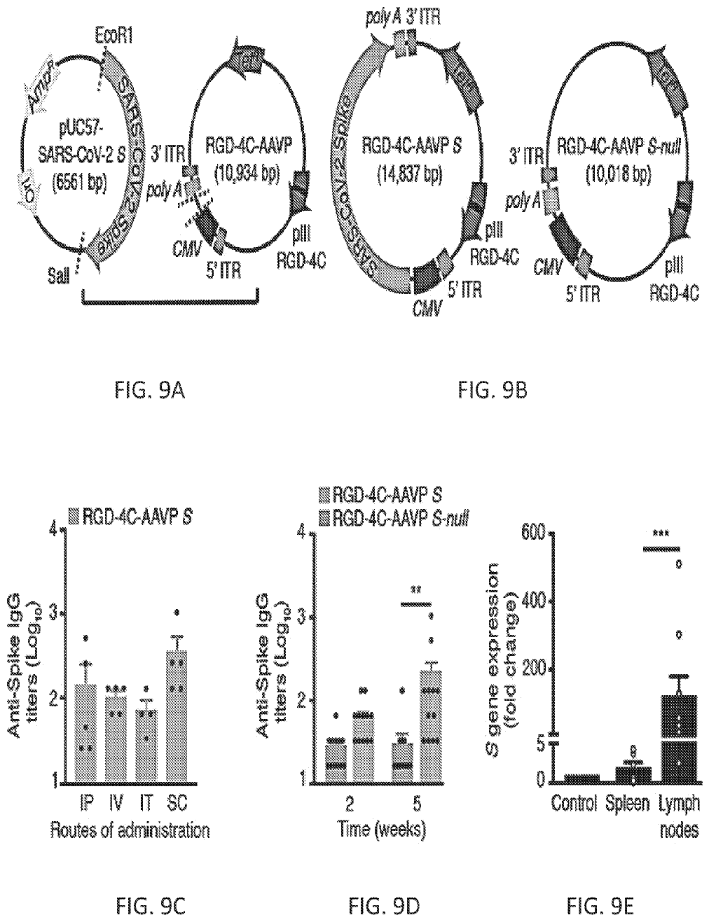

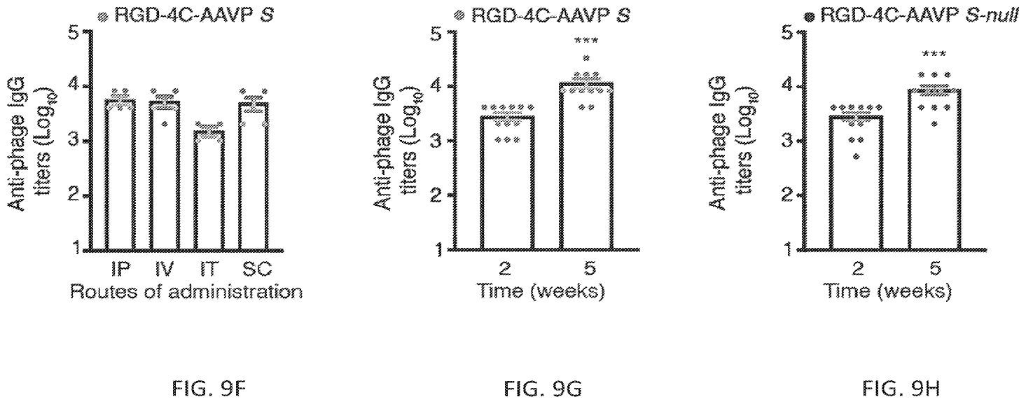

As a parallel approach to the dual-display phage design, the AAVP platform of gene delivery was adapted to generate a novel AAVP-based vaccine candidate for SARS-CoV-2. Thus, targeted AAVP particles encoding the full-length S protein gene (AAVP 5) (Wuhan-Hu-1 strain, GenBank Accession Number: NC_045512.2) were designed and generated that display the ACDCRGDCFCG (RGD4C) (SEQ ID NO: 5) ligand peptide on the pIII minor coat protein that targets αv integrins which are known to regulate the trafficking of lymphocytes and antigen-presenting cells (i.e., dendritic cells) into secondary lymphoid organs. As a control, an AAVP empty vector that contains all the phage and AAV elements, except the S gene (AAVP S-null) was generated (FIG. 9A, B).

To assess the immunogenicity of RGD4C-AAVP S, five-week-old female outbred Swiss Webster mice were immunized. Cohorts of mice (n=5) received 10⁹ TU of targeted RGD4C-AAVP S via intraperitoneal (group 1), intravenous (group 2), intratracheal (group 3), or subcutaneous (group 4) routes. S protein-specific IgG antibody response was evaluated 14 days later by ELISA. Baseline sera were used as controls (FIG. 9C). The administration of RGD4C-AAVP S particles elicited serum IgG responses against the S protein in all experimental groups, whereas mice immunized via the subcutaneous route had higher serum IgG titers than the other groups. Thus, it was elected to administer RGD4C-AAVP S via subcutaneously in the subsequent in vivo assays.

Next, the immunization regimen was tested in five-week-old female inbred BALB/c mice. Cohorts of mice (n=12 per group) received weekly doses of 10⁹ TU of RGD4C-AAVP S or the control, RGD4C-AAVP S-null subcutaneously. Higher titers of S protein-specific IgG antibodies were observed in mice vaccinated with RGD4C-AAVP S relative to the baseline sera and the RGD4C-AAVP S-null, especially five weeks post-vaccination, confirming that RGD4C-AAVP S is a suitable vector for transgene delivery and elicits a systemic humoral immune response (FIG. 9D).

To gain insight into the S protein transgene expression mediated by RGD4C-AAVP S, the fate of the transduced genome in mice tissues after 28 days of immunization was investigated, including the main regional lymph nodes (axillary, inguinal, mesenteric, and mediastinal nodes). Notably, transgene expression was detected at different levels exclusively in the draining lymph nodes. Skeletal muscle and spleen were used as control organs and did not show detectable transgene expression (FIG. 9E). As expected, no S protein transgene was detected in mice immunized with RGD4C-AAVP S-null. These results confirm that gene delivery by AAVP and the expression of the S protein in the draining lymph nodes triggers a systemic S protein-specific humoral response. Moreover, the data recapitulate the well-established attributes of AAVP particles in eliminating off-target effects, even upon clearance via the reticulum-endothelial system (RES), sparing non-targeted or distal tissues, while a strong promoter drives the expression of the transgene in the transduced cells. This finding is particularly important for evaluating potential side effects in novel, candidate AAVP-based vaccines, since off-target effects have been reported in many toxicological studies of adenovirus vaccines. Therefore, the extensive body of data generated to date with AAVP in cancer gene therapies can help accelerate future clinical development of AAVP vaccine candidates, potentially saving time and resources.

Investigations also examined the antibody response against phage in mice vaccinated with RGD4C-AAVP S or the RGD4C-AAVP S-null phage. A strong and sustained phage-specific IgG antibody response was observed upon administration of RGD4C-AAVP S by all routes of administration (FIG. 9F), and a significantly higher response in the group of mice administered subcutaneously (FIG. 9G). Likewise, mice administered with the RGD4C-AAVP S-null phage also developed phage-specific IgG responses (FIG. 9H), indicating that AAVP are strong immunogens and can serve as important adjuvants for AAVP-based vaccination. Together, these results suggest that targeted AAVP S is an efficient tool for transgene expression and can induce an effective immune response against the viral S protein.

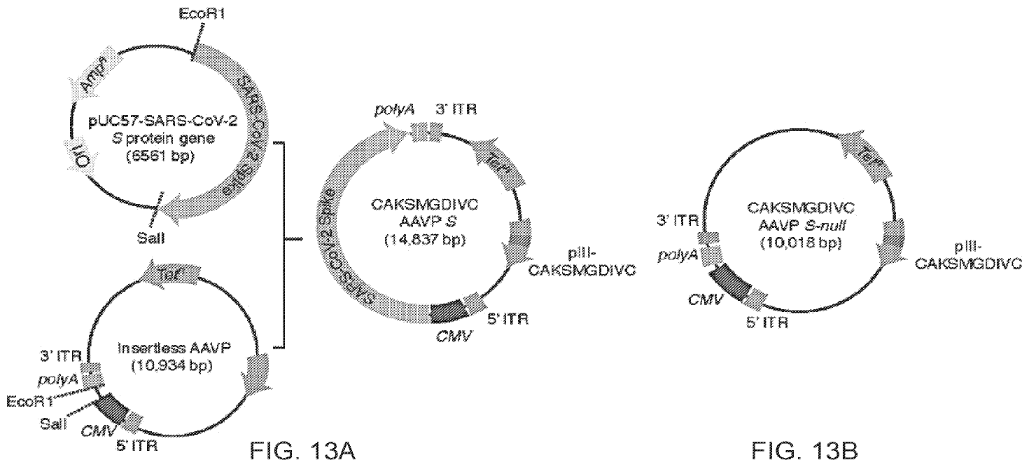

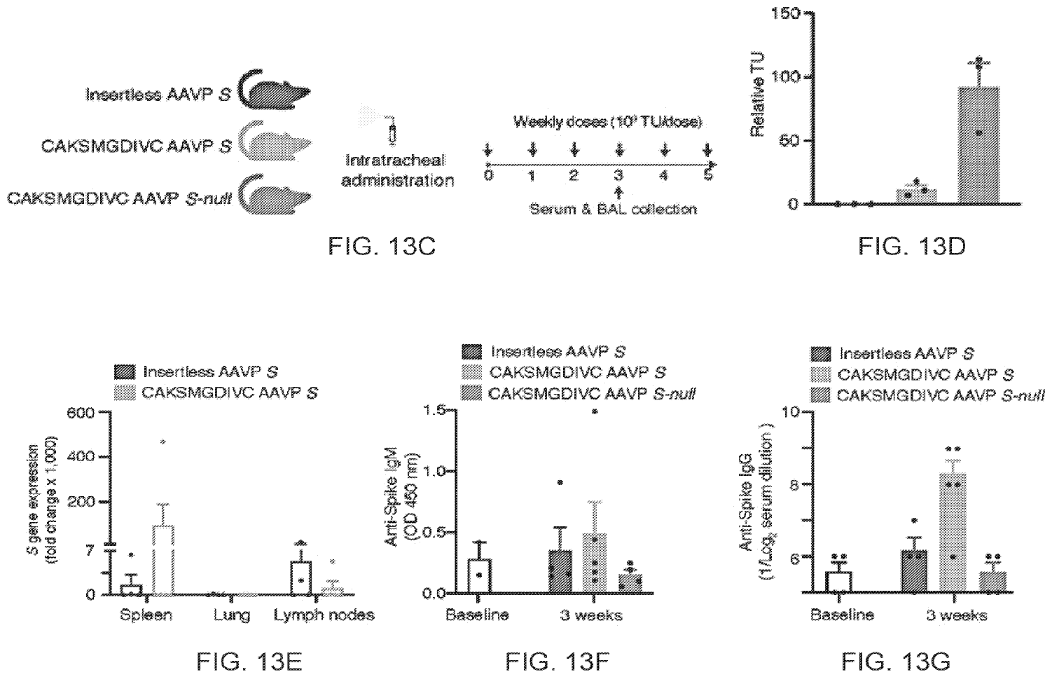

In addition, AAVP particles that display the CAKSMGDIVC (SEQ ID NO: 4) ligand peptide on the pIII minor coat protein were designed and generated to use as a lung epithelium-targeted motif. As controls, we used an insertless-AAVP S which contains the Spike (S) encoding transgene but lacks a ligand sequence on the pIII and the CAKSMGDIVC-AAVP transgene null vector that contains all the phage and AAV elements, except the S gene (AAVP S-null) (FIG. 13A, 13B). Investigations also examined the receptor-mediated transport of CAKSMGDIVC-AAVP S or CAKSMGDIVC-AAVP S-null across the lung into the bloodstream after 1 h. (FIG. 13C) As expected, both CAKSMGDIVC-targeted AAVP constructs cross the lung barriers, recapitulating the features of the CAKSMGDIVC ligand peptide to allow receptor-mediated transport of phage and AAVP particles. Transgene expression was detected at different levels in the spleen and upper lymph nodes, suggesting that the presence of CAKSMGDIVC-AAVP S in the bloodstream facilitates the induction of systemic immune response. Skeletal muscle was used as a control organ and did not show detectable transgene expression (FIG. 13D). No S gene expression was detected in mice immunized with CAKSMGDIVC-AAVP S-null. These results confirm that gene delivery by AAVP and the expression of the S protein in the spleen and draining lymph nodes trigger a systemic S protein-specific humoral response. The administration of CAKSMGDIVC-AAVP S particles elicited serum IgM (FIG. 13F) and IgG (FIG. 13G) responses against the S protein.

### Example 8: Discussion

In present disclosure phage- and AAVP-based vaccine candidates were designed, generated, and evaluated for translational potential using epitope display and gene delivery as strategies against SARS-CoV-2. It was demonstrated that both strategies can be successfully used to induce an antigen-specific or polyclonal humoral response, respectively, against the S protein and therefore represent valid approaches for vaccine development.

One of the main challenges associated with the current vaccines is to predict the potency of the immune response toward protective epitopes on the S protein especially in the face of new genetic variants. In principle, focusing on structural antigen mapping and immunodominant B- and T-cell epitopes that trigger protective immune responses associated with potent neutralizing activity would lead to long-term protective vaccines. As such, many studies have been dedicated to predict epitopes from B- and T-cells derived from the SARS-CoV-2 S protein and other structural proteins. Similarly, six exposed regions of the S protein were selected with specific structural constraints for display on the phage capsid and to increase the likelihood of antigen recognition and processing by the host immune system. It was found that epitope 4 (SEQ ID NO: 25) triggered a strong and specific systemic humoral response against the S protein, presumably by recapitulating the near-native conformation of the epitope when expressed on rpVIII major capsid protein. Of note, epitope 4 is unchanged in three main emergent SARS-CoV-2 viral lineages: Alpha, first identified in the U. K. (Thomson et al., 2021), Beta, first identified in South Africa (Tegally et al., 2020), and Gamma, first identified in Brazil (Faria et al., 2021). Thus, the combinatorial approach of selecting regions of an antigen-based on conformational constraints and evaluating their structural properties in silico can identify the epitopes most likely to replicate the natural immune response to an infection. Without wishing to be bound by theory, this observation suggests that antigen-engineering strategies, such as the vaccine candidate for the Zika virus, have the potential to generate vaccines with high efficacy in producing neutralizing antibodies and cell-mediated response.

To support the translational application of a phage-based vaccine, a protocol of immunization in mice was designed as a proof-of-principle towards pulmonary vaccination against SARS-CoV-2. The design of dual-display phage particles simultaneously displaying both epitope 4 (SEQ ID NO: 25) on the recombinant major capsid pVIII protein and the CAKSMGDIVC (SEQ ID NO: 4) targeting ligand, responsible for selective targeting and transport of phage particles to the systemic circulation, on the minor pIII coat protein confirmed that an aerosol strategy of immunization against SARS-CoV-2 may confer remarkable advantages over conventional routes of immunization. First, unlike subcutaneous or intramuscular injections, inhalation is needle-free, and thus requires minimal use of specialized medical staff for administration. Moreover, the large and accessible lung surface with highly vascularized pulmonary epithelium is a unique feature that is known to induce local immune protection against airborne pathogens. Emerging studies of intranasal or intratracheal immunizations are showing successful protection against SARS-CoV-2 challenge in mice and non-human primates. Of course, it is also contemplated that the targeted phage-based aerosol formulations of the present disclosure can be delivered by suitable devices, such as portable inhalers (e.g., commercially available pressurized metered-dose inhalers, dry powder inhalers, and nebulizers), to produce particles of optimal size and mass for proper lung deposition in human patients.

The presently described studies also uncovered the potential value of AAVP to deliver the S gene as an alternative SARS-CoV-2 vaccine candidate. In the last decade, AAVP technology has proven to be a modular platform that can be appropriately tailored to image and treat a variety of human solid tumors in mouse models and spontaneous tumors in pet dogs. These attributes make AAVP a unique platform for gene delivery. Indeed, the present studies showed that administration of targeted AAVP S particles elicited an antibody response against the encoded transgene, S protein. Because the prototype AAVP S vaccine is targeted with the integrin-binding peptide (RGD4C), which has a high affinity binding for αv integrins, this may facilitate the transduction of inflammatory cells trafficking to the lymph nodes where gene expression and antigen presentation takes place. In addition, the identification of the RGD motif within the RBD domain of the S protein suggests that integrins may act as a co-receptor or alternate path for viral entry. Therefore, it is plausible that different functional ligands for tissue-specific transgene expression within lymph nodes, lymphatic vessels, or lung epithelial cells (e.g., CAKSMGDIVC; SEQ ID NO: 4) followed by systemic delivery may enhance the efficacy and broad administration of AAVP-based vaccines.

In conclusion, the studies of the present disclosure show that phage particles are highly effective tools for the development of phage- or AAVP-based vaccines against SARS-CoV-2 S protein-specific humoral responses. Additionally, the process of conducting the studies of the present disclosure necessitated the development and optimization of Good Manufacturing Practices (GMP) for the generation, production, and purification of engineered phage particles. These processes can now be applied on an industrial, large-scale level for rapid commercialization.

### Enumerated Embodiments

The following enumerated embodiments are provided, the numbering of which is not to be construed as designating levels of importance.

Embodiment 1 provides an immunogenic composition comprising an effective amount of a therapeutic engineered phage and a pharmaceutically acceptable carrier, wherein the therapeutic engineered phage comprises one or more fusion polypeptides comprising an antigenic polypeptide and a phage coat protein.

Embodiment 2 provides the immunogenic composition of embodiment 1, wherein the therapeutic engineered phage further comprises a fusion polypeptide comprising a tissue-targeting polypeptide and a phage coat protein.

Embodiment 3 provides the immunogenic composition of any one of embodiments 1 and 2, wherein the phage coat protein selected from the group comprising pIII protein, pVI protein, pVII protein, pVIII protein, rpVIII protein, and pIX protein.

Embodiment 4 provides the immunogenic composition of any one of embodiments 2-3, wherein the tissue-targeting polypeptide targets lymph node tissue.

Embodiment 5 provides the immunogenic composition of embodiment 4, wherein the lymph-node tissue targeting polypeptide comprises an amino acid sequence selected from the group comprising SEQ ID NOs: 1-2.

Embodiment 6 provides the immunogenic composition of claim 4, wherein the lymph-node tissue targeting polypeptide is encoded by a nucleotide sequence selected from the group comprising SEQ ID NOs: 7-8.

Embodiment 7 provides the immunogenic composition of any one of embodiments 2-3, wherein the tissue-targeting polypeptide targets lymphatic channel tissue.

Embodiment 8 provides the immunogenic composition of embodiment 7, wherein the lymphatic channel tissue targeting polypeptide comprises an amino acid sequence comprising SEQ ID NO: 3.

Embodiment 9 provides the immunogenic composition of embodiment 7, wherein the lymphatic channel tissue targeting polypeptide is encoded by a nucleotide sequence comprising SEQ ID NO: 9.

Embodiment 10 provides the immunogenic composition of any one of embodiments 2-3, wherein the tissue-targeting polypeptide targets lung tissue.

Embodiment 11 provides the immunogenic composition of embodiment 10, wherein the lung tissue targeting polypeptide comprises an amino acid sequence selected from the group comprising SEQ ID NO: 4 and 28.

Embodiment 12 provides the immunogenic composition of any one of embodiments 2-3, wherein the tissue-targeting polypeptide is an integrin-binding domain.

Embodiment 13 provides the immunogenic composition of embodiment 12, wherein the integrin-binding polypeptide comprises an amino acid sequence selected from the group comprising SEQ ID NO: 4, 5, and 86.

Embodiment 14 provides the immunogenic composition of embodiment 12, wherein the integrin-binding polypeptide is encoded by a nucleotide sequence selected from the group comprising SEQ ID NOs: 6 and 81.

Embodiment 15 provides the immunogenic composition of any one of embodiments 2-3, wherein the tissue-targeting polypeptide is a GRP78-binding domain.

Embodiment 16 provides the immunogenic composition of embodiment 15, wherein the GRP78-binding polypeptide comprises an amino acid sequence selected from the group comprising SEQ ID NOs: 29 and 30.

Embodiment 17 provides the immunogenic composition of any one of embodiments 1, 20-26, wherein the therapeutic engineered phage further comprises a fusion polypeptide comprising an aerosol delivery polypeptide that targets lung tissue and acts as a transcytosis domain and a phage coat protein.

Embodiment 18 provides the immunogenic composition of embodiment 17, wherein the aerosol delivery polypeptide comprises the amino acid sequence of SEQ ID NO: 4.

Embodiment 19 provides the immunogenic composition of embodiment 17, wherein the aerosol delivery peptide is encoded by a nucleic acid sequence comprising SEQ ID NO: 81

Embodiment 20 provides the immunogenic composition of any one of embodiments 1-19, wherein the antigenic polypeptide is a viral polypeptide.

Embodiment 21 provides the immunogenic composition of embodiment 20, wherein the viral polypeptide is an epitope derived from a viral protein selected from the group comprising a coronavirus S protein, a coronavirus N protein, a coronavirus M protein, and a coronavirus E protein.

Embodiment 22 provides the immunogenic composition of embodiment 21, wherein the epitope is at least one selected from SEQ ID NOs: 10-27, 31-80, 111, 120, 124, 126, 135, and 136.

Embodiment 23 provides the immunogenic composition of any one of embodiments 1-22, wherein the therapeutic engineered phage is an adeno-associated viral bacteriophage (AAVP) and further comprises a viral gene.

Embodiment 24 provides the immunogenic composition of claim 23, wherein the viral gene is selected from the group comprising a coronavirus S protein, a coronavirus N protein, a coronavirus M protein, and a coronavirus E protein.

Embodiment 25 provides the immunogenic composition of any one of embodiments 23 and 24, wherein the viral gene is a coronavirus S protein and encodes an amino acid sequence selected from the group consisting of SEQ ID NOs: 83 and 85.

Embodiment 26 provides the immunogenic composition of any one of embodiments 23 and 24, wherein the viral gene is a coronavirus S protein and comprises a nucleotide sequence selected from the group consisting of SEQ ID NOs: 82 and 84.

Embodiment 27 provides a nucleic acid vector comprising the immunogenic composition of any one of embodiments 1-26.

Embodiment 28 provides the nucleic acid vector of embodiment 27, wherein the vector comprises an antigenic polypeptide-pVIII coat protein fusion protein encoding sequence, and tissue-targeting polypeptide-pIII coat protein fusion protein encoding sequence.

Embodiment 29 provides the nucleic acid vector of embodiment 27, wherein the vector comprises a tissue-targeting polypeptide-pVIII or rpVIII coat protein fusion protein encoding sequence and an antigenic polypeptide-containing-pIII coat protein fusion protein encoding sequence.

Embodiment 30 provides the nucleic acid vector of embodiment 27, wherein the vector comprises an antigenic polypeptide-pVIII or rpVIII coat protein fusion protein encoding sequence.

Embodiment 31 provides the nucleic acid vector of embodiment 27, wherein the vector comprises an antigenic polypeptide-containing-pIII coat protein fusion protein encoding sequence.

Embodiment 32 provides the nucleic acid vector of embodiment 27, wherein the vector comprises a 5′ ITR, a CMV promoter, an antigenic polypeptide encoding sequence, a poly-A sequence, a 3′ ITR, and a tissue-targeting polypeptide-pIII coat protein fusion protein-encoding sequence.

Embodiment 33 provides the nucleic acid vector of embodiment 27, wherein the vector comprises a 5′ ITR, a CMV promoter, an antigenic polypeptide encoding sequence, a poly-A sequence, a 3′ ITR, a Tac promoter, a tissue-targeting polypeptide-pVIII or rpVIII coat protein fusion protein encoding sequence, and an aerosol delivery polypeptide-pIII coat protein fusion protein encoding sequence.

Embodiment 34 provides the nucleic acid vector of embodiment 27, wherein the vector comprises a 5′ ITR, a CMV promoter, an antigenic polypeptide encoding sequence, a poly-A sequence, a 3′ ITR, a Tac promoter, an aerosol-delivery polypeptide-pVIII or rpVIII coat protein fusion protein encoding sequence, and a tissue-targeting polypeptide-pIII coat protein encoding sequence.

Embodiment 35 provides a method of stimulating an immune response in a subject, the method comprising administering to the subject one or more of the immunogenic compositions of any one of embodiments 1-26.

Embodiment 36 provides the method of embodiment 35, wherein the one or more immunogenic compositions are delivered by a route selected from the group comprising oral route, inhalation route, nasal route, nebulization route, intratracheal route, intravenous injection, intraperitoneal injection, intramuscular injection, subcutaneous injection, and transdermal injection.

Embodiment 37 provides a method for treating, ameliorating, and/or preventing a coronavirus infection in a subject, comprising administering an effective amount of one or more of the immunogenic compositions of any one of embodiments 1-26.

Embodiment 38 provides the method of embodiment 37, wherein the one or more immunogenic compositions are delivered by a route selected from the group comprising oral route, inhalation route, nasal route, nebulization route, intratracheal, intravenous injection, intraperitoneal injection, intramuscular injection, subcutaneous injection, and transdermal injection.

Embodiment 39 provides the method of any one of embodiments 37 and 38, wherein the coronavirus infection is caused by a coronavirus selected from the group comprising SARS-CoV, SARS-CoV-2, HCoV-229E, HCoV-NL63, MERS-CoV, HCoV-OC43, HCoV-HKU1, and murine hepatitis virus, type 1 (MHV-1).

Embodiment 40 provides a method of promoting gene delivery to a virally-infected cell, the method comprising contacting the cell with a therapeutic engineered phage comprising a fusion protein comprising a ligand-binding polypeptide and a phage coat protein.

Embodiment 41 provides the method of embodiment 40, wherein the phage coat protein is selected from the group comprising pIII protein, pVI protein, pVII protein, pVIII protein, rpVIII protein, and pIX protein.

Embodiment 42 provides the method of any one of embodiments 40 and 41, wherein the ligand-binding polypeptide is selected from the group comprising SEQ ID NOs: 1-5, 28-30, and 86.

Embodiment 43 provides the method of any one of embodiments 40-42, wherein the therapeutic engineered phage is an adeno-associated virus/phage (AAVP).

Embodiment 44 provides a method of treating, ameliorating, and/or preventing a viral infection in a subject, comprising administering an effective amount of a therapeutic engineered phage comprising a fusion protein comprising a ligand-binding polypeptide and a phage coat protein, thereby treating, ameliorating, and/or preventing the viral infection.

Embodiment 45 provides the method of embodiment 44, wherein the phage coat protein selected from the group comprising pIII protein, pVI protein, pVII protein, pVIII protein, rpVIII protein, and pIX protein.

Embodiment 46 provides the method of any one of embodiments 44 and 45, wherein the ligand-binding polypeptide is selected from the group comprising SEQ IDs: 1-5, 28-30, and 86.

Embodiment 47 provides the method of any one of embodiments 44-46, wherein the ligand-binding polypeptide is a GRP78-binding domain.

Embodiment 48 provides the method of embodiment 47, wherein the GRP78-binding polypeptide comprises the amino acid sequence selected from the group comprising SEQ ID NOs: 29 and 30.

Embodiment 49 provides the method of any one of embodiments 44-48, wherein the therapeutic engineered phage is an adeno-associated virus/phage (AAVP).

Embodiment 50 provides the method of any one of embodiments 44-49, wherein the therapeutic engineered phage further comprises an anti-viral agent.

Embodiment 51 provides the method of embodiment 50, wherein the anti-viral agent is selected from the group comprising an anti-viral drug or precursor thereof, an anti-viral polypeptide or precursor thereof, and an anti-viral nucleic acid.

### Other Embodiments

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this disclosure has been disclosed with reference to specific embodiments, it is apparent that other embodiments and variations of this disclosure may be devised by others skilled in the art without departing from the true spirit and scope of the disclosure. The appended claims are intended to be construed to include all such embodiments and equivalent variations.

1. An immunogenic composition comprising an effective amount of a therapeutic engineered phage and a pharmaceutically acceptable carrier, wherein the therapeutic engineered phage comprises one or more fusion polypeptides comprising an antigenic polypeptide and a phage coat protein.

2. The immunogenic composition of claim 1, wherein the therapeutic engineered phage further comprises a fusion polypeptide comprising a tissue-targeting polypeptide and a phage coat protein.

3. The immunogenic composition of claim 1, wherein the phage coat protein comprises at least one of pIII protein, pVI protein, pVII protein, pVIII protein, rpVIII protein, and pIX protein.

4. The immunogenic composition of claim 2, wherein the tissue-targeting polypeptide targets lymph node tissue.

5. The immunogenic composition of claim 4, wherein the lymph node tissue targeting polypeptide comprises an amino acid sequence selected from the group comprising SEQ ID NOs: 1-2.

6. The immunogenic composition of claim 4, wherein the lymph node tissue targeting polypeptide is encoded by a nucleotide sequence selected from the group comprising SEQ ID NOs: 7-8.

7. The immunogenic composition of claim 2, wherein the tissue-targeting polypeptide targets lymphatic channel tissue.

8. The immunogenic composition of claim 7, wherein the lymphatic channel tissue targeting polypeptide comprises an amino acid sequence comprising SEQ ID NO: 3.

9. The immunogenic composition of claim 7, wherein the lymphatic channel tissue targeting polypeptide is encoded by a nucleotide sequence comprising SEQ ID NO: 9.

10. The immunogenic composition of claim 2, wherein the tissue-targeting polypeptide targets lung tissue.

11. The immunogenic composition of claim 10, wherein the lung tissue targeting polypeptide comprises an amino acid sequence selected from the group comprising SEQ ID NOs: 4 and 28.

12. The immunogenic composition of claim 2, wherein the tissue-targeting polypeptide is an integrin-binding domain.

13. The immunogenic composition of claim 12, wherein the integrin-binding polypeptide comprises an amino acid sequence selected from the group comprising SEQ ID NOs: 4, 5, and 86.

14. The immunogenic composition of claim 12, wherein the integrin-binding polypeptide is encoded by a nucleotide sequence selected from the group comprising SEQ ID NOs: 6 and 81.

15. The immunogenic composition of claim 2, wherein the tissue-targeting polypeptide is a GRP78-binding domain.

16. The immunogenic composition of claim 15, wherein the GRP78-binding polypeptide comprises an amino acid sequence selected from the group comprising SEQ ID NOs: 29 and 30.

17. The immunogenic composition of claim 1, wherein the antigenic polypeptide is a viral polypeptide.

18. The immunogenic composition of claim 17, wherein the viral polypeptide is an epitope derived from a viral protein selected from the group comprising a coronavirus S protein, a coronavirus N protein, a coronavirus M protein, and a coronavirus E protein.

19. The immunogenic composition of claim 18, wherein the epitope is at least one from the group comprising SEQ ID NOs: 10-27, 31-80, 111, 120, 124, 126, 135, and 136.

20. The immunogenic composition of claim 1, wherein the therapeutic engineered phage is an adeno-associated viral bacteriophage (AAVP) and further comprises a viral gene.

21. The immunogenic composition of claim 20, wherein the viral gene is selected from the group comprising a coronavirus S protein, a coronavirus N protein, a coronavirus M protein, and a coronavirus E protein.

22. The immunogenic composition of claim 20, wherein the viral gene is a coronavirus S protein and encodes an amino acid sequence selected from the group consisting of SEQ ID NOs: 83 and 85.

23. The immunogenic composition of claim 20, wherein the viral gene is a coronavirus S protein and comprises a nucleotide sequence selected from the group consisting of SEQ ID NOs: 82 and 84.

24. The immunogenic composition of claim 20, wherein the therapeutic engineered phage further comprises a fusion polypeptide comprising an aerosol delivery polypeptide that targets lung tissue and acts as a transcytosis domain and a phage coat protein.

25. The immunogenic composition of claim 24, wherein the aerosol delivery polypeptide comprises the amino acid sequence of SEQ ID NO: 4.

26. The immunogenic composition of claim 24, wherein the aerosol delivery peptide is encoded by a nucleic acid sequence comprising SEQ ID NO: 81.

27. A nucleic acid vector comprising the immunogenic composition of claim 1.

28. The nucleic acid vector of claim 27, wherein the vector comprises an antigenic polypeptide-pVIII or rpVIII coat protein fusion protein encoding sequence and a tissue-targeting polypeptide-pIII coat protein fusion protein encoding sequence.

29. The nucleic acid vector of claim 27, wherein the vector comprises a tissue-targeting polypeptide-pVIII or rpVIII coat protein fusion protein encoding sequence, and an antigenic polypeptide-containing-pIII coat protein fusion protein encoding sequence.

30. The nucleic acid vector of claim 27, wherein the vector comprises an antigenic polypeptide-pVIII or rpVIII coat protein fusion protein encoding sequence.

31. The nucleic acid vector of claim 27, wherein the vector comprises an antigenic polypeptide-containing-pIII coat protein fusion protein encoding sequence.

32. The nucleic acid vector of claim 27, wherein the vector comprises a 5′ ITR, a CMV promoter, an antigenic polypeptide encoding sequence, a poly-A sequence, a 3′ ITR, and a tissue-targeting polypeptide-pIII coat protein fusion protein-encoding sequence.

33. The nucleic acid vector of claim 27, wherein the vector comprises a 5′ ITR, a CMV promoter, an antigenic polypeptide encoding sequence, a poly-A sequence, a 3′ ITR, a Tac promoter, a tissue-targeting polypeptide-pVIII or rpVIII coat protein fusion protein encoding sequence, and an aerosol delivery polypeptide-pIII coat protein fusion protein encoding sequence.

34. The nucleic acid vector of claim 27, wherein the vector comprises a 5′ ITR, a CMV promoter, an antigenic polypeptide encoding sequence, a poly-A sequence, a 3′ ITR, a Tac promoter, an aerosol-delivery polypeptide-pVIII or rpVIII coat protein fusion protein encoding sequence, and a tissue-targeting polypeptide-pIII coat protein encoding sequence.

35. A method of stimulating an immune response in a subject, the method comprising administering to the subject one or more of the immunogenic compositions of claim 1.

36. The method of claim 35, wherein the one or more immunogenic compositions are delivered by a route selected from the group comprising oral, inhalation, nasal, nebulization, intratracheal, intravenous, intraperitoneal, intramuscular, subcutaneous, and transdermal.

37. A method for treating, ameliorating, or preventing a coronavirus infection in a subject, the method comprising administering an effective amount of the immunogenic composition of claim 1.

38. The method of claim 37, wherein the one or more immunogenic compositions are delivered by a route selected from the group comprising oral, inhalation, nasal, nebulization, intratracheal, intravenous, intraperitoneal, intramuscular, subcutaneous, and transdermal.

39. The method of claim 37, wherein the coronavirus infection is caused by a coronavirus selected from the group comprising SARS-CoV, SARS-CoV-2, HCoV-229E, HCoV-NL63, MERS-CoV, HCoV-OC43, HCoV-HKU1, and murine hepatitis virus, type 1 (MHV-1).

40. A method of promoting gene delivery to a virally-infected cell, the method comprising contacting the cell with a therapeutic engineered phage comprising a fusion protein comprising a ligand-binding polypeptide and a phage coat protein.

41. The method of claim 40, wherein the phage coat protein is at least one of pIII protein, pVI protein, pVII protein, pVIII protein, rpVIII protein, and pIX protein.

42. The method of claim 40, wherein the ligand-binding polypeptide is selected from the group comprising SEQ ID NOs: 1-5, 28-30, and 86.

43. The method of claim 40, wherein the therapeutic engineered phage is an adeno-associated virus/phage (AAVP).

44. A method of treating, ameliorating, or preventing a viral infection in a subject, the method comprising administering to the subject an effective amount of a therapeutic engineered phage comprising a fusion protein comprising a ligand-binding polypeptide and a phage coat protein, thereby treating, ameliorating, or preventing the viral infection in the subject.

45. The method of claim 44, wherein the phage coat protein is at least one of pIII protein, pVI protein, pVII protein, pVIII protein, rpVIII protein and pIX protein.

46. The method of claim 44, wherein the ligand-binding polypeptide is selected from the group comprising SEQ IDs: 1-5, 28-30, and 86.

47. The method of claim 44, wherein the ligand-binding polypeptide is a GRP78-binding domain.

48. The method of claim 47, wherein the GRP78-binding polypeptide comprises an amino acid sequence selected from the group comprising SEQ ID NOs: 29 and 30.

49. The method of claim 44, wherein the therapeutic engineered phage is an adeno-associated virus/phage (AAVP).

50. The method of claim 44, wherein the therapeutic engineered phage further comprises an anti-viral agent.

51. The method of claim 50, wherein the anti-viral agent is selected from the group comprising an anti-viral drug or precursor thereof, an anti-viral polypeptide or precursor thereof, and an anti-viral nucleic acid.
